(19) United States
(12) Patent Application Publication
(10) Pub. No.: US 2012/0010281 A1
(43) Pub. Date: Jan. 12, 2012

(54) ANTIMICROBIAL AND ANTITUBERCULAR COMPOUNDS

(75) Inventors: Erik J. Sorensen, Princeton, NJ (US); Stephen D. Lotesta, Burlington, NJ (US); Junjia Liu, Princeton, NJ (US); Emma V. Yates, Coconut Creek, FL (US); Maurice A. Marsini, Hopewell, NJ (US)

(73) Assignee: THE TRUSTEES OF PRINCETON UNIVERSITY, Princeton, NJ (US)

(21) Appl. No.: 13/166,468

(22) Filed: Jun. 22, 2011

Related U.S. Application Data
(60) Provisional application No. 61/357,306, filed on Jun. 22, 2010.
(60) Provisional application No. 61/407,338, filed on Oct. 27, 2010.
(60) Provisional application No. 61/451,851, filed on Mar. 11, 2011.
(60) Provisional application No. 61/470,189, filed on Mar. 31, 2011.

(51) Int. Cl. A61K 31/343; A61P 31/06; A61K 31/122; A61P 31/00; C07C 45/00; C07D 317/72

(52) U.S. Cl. 514/462; 568/361; 549/341; 514/691

(57) ABSTRACT

Infections caused by Mycobacterium tuberculosis kill more than 1.8 million people each year. While the persistence of this pathogenic bacterial species and the emergence of multidrug resistant strains have created an urgent need for new TB therapies, a new TB-specific drug has not been developed in over 40 years. The disclosure herein provides short and scalable syntheses of small molecules, and small molecules as new therapeutics for eradicating this life threatening pathogen.

### CROSS REFERENCE TO RELATED APPLICATIONS

This application claims the benefit of U.S. provisional application No. 61/357,306, filed Jun. 22, 2010; U.S. provisional application No. 61/407,338, filed Oct. 27, 2010; U.S. provisional application No. 61/451,851 filed Mar. 11, 2011; and U.S. provisional application No. 61/470,189 filed Mar. 31, 2011, which are all incorporated herein by reference as if fully set forth.

### GOVERNMENT SUPPORT STATEMENT

This invention was made with government support under Grant #GM065483 awarded by the National Institutes of Health. The government has certain rights in this invention.

### FIELD OF INVENTION

The disclosure herein relates to anti-microbial compounds, methods of making anti-microbial compounds and methods of treating disease with anti-microbial compounds.

### BACKGROUND

Pleuromutilin (3, below) was first isolated in 1951 from the basidiomycetes fungus Pleurotus mutilus. Structurally, pleuromutilin possesses a rigid, propellane-like 8-6-5 tricyclic carbon skeleton including eight stereocenters. Seven of these are contiguous, and three are all-carbon quaternary stereocenters. With the exception of two modifications, it is still largely undeveloped in the context of antibacterial drug production. It is likely that the numerous dimensions of chemical complexity within pleuromutilin have impeded the development of new antibiotics based on its structure.

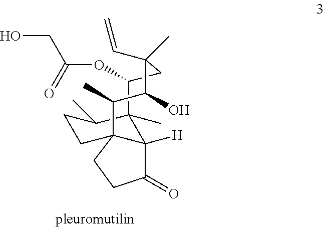

Pleuromutilin was first shown to selectively inhibit bacterial protein synthesis in prokaryotic ribosomes, and has been found to exhibit promising levels of antibacterial activity (μM range) against a number of bacterial cell lines. Unfortunately, this intriguing compound suffers from insufficient in vivo potency due to rapid metabolic degradation by cytochrome P-450. Several semi-synthetic analogs of pleuromutilin have been identified and developed for use as anti-bacterial and anti-tubercular agents. Through systematic chemical modifications of pleuromutilin, scientists at GlaxoSmithKline created the pleuromutilin relative retapamulin (45). This semi-synthetic derivative of pleuromutilin (3) is significant because it targets the 50S subunit of the bacterial ribosome, yet is unaffected by resistance to other 50S-targeting classes of antibiotics such as the erythromycins. The C11 hydroxyl and the C3 and C21 carbonyls of pleuromutilin have been shown to be important for activity of the compound. Another pleuromutilin relative developed is tiamulin.

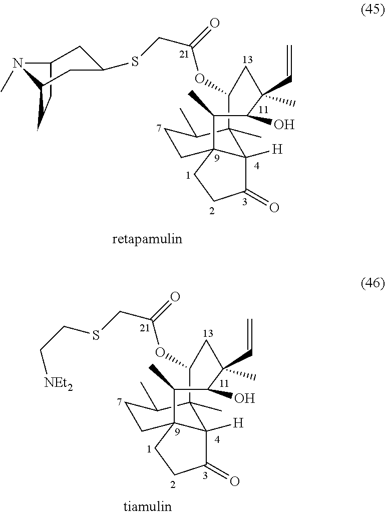

### SUMMARY

In an aspect, the invention relates to a method of synthesizing a pleuromutilin analog. The method includes providing a pleuromutilin skeleton having the structure of one of formulas 6-1, 6-2 or 6-3:

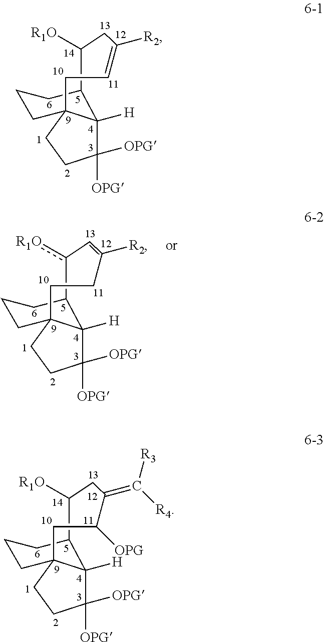

the C14-O bond in formulas 6-1, 6-2, and 6-3 and the C11-O bond in formula 6-3 are independently selected from any stereoisomer orientation. R₁ is selected from the group consisting of H, H and a hydroxyl protecting group, any one of the diverse pleuromutilin or pleuromutilin derivative side-chains, and any one of the diverse pleuromutilin or pleuromutilin derivative side-chains having a protecting group. PG is a hydroxyl protecting group. PG′ is a ketone protecting group. R2, R3 and R4 are independently selected from the group consisting of H, CH₃, an alkyl group, alkenyl group and an aryl group. The method also includes one or more of i) introducing any one of the diverse pleuromutilin or pleuromutilin derivative side-chains as a C21 acyl side chain, ii) introducing one or more pleuromutilin analog substituent, and iii) conducting two fold de-protection to form a C3 ketone and unveil the C11 hydroxyl.

In an aspect, the invention relates to a composition including a pleuromutilin analog having a structure of one formulas 7-1 or 7-2:

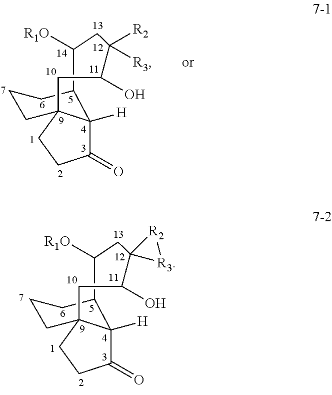

The bonds on C11, C12 or C14 are independently selected from any stereoisomer orientation. For the pleuromutilin analog having the structure of formula 7-1, R₁ is selected from the group consisting of any one of the diverse pleuromutilin or pleuromutilin derivative side-chains as a C21 acyl side chain; R₂ is selected from the group consisting of H, OH, N and O; R₃ is selected from the group consisting of CH₂OH and CH₂X and X is selected from the group consisting of a halogen atom, a nitrogen atom, an alkyl group, an alkenyl group and an aryl group. For the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R1 is selected from the group consisting of O, HN, HCH3, Net, NPr and NBu, and R3 is selected from the group consisting of alkylene groups.

In an aspect, the invention relates to a pharmaceutical composition comprising a pleuromutilin analog or a pharmaceutically acceptable salt or solvate thereof. The pleuromutilin analog has the structure of one of formulas 7-1 or 7-2:

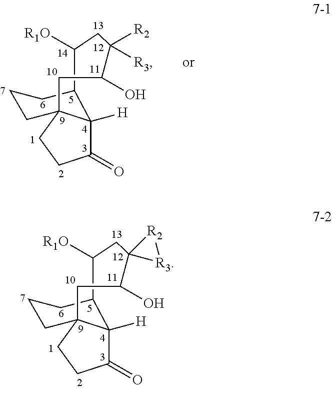

The bonds on C11, C12 or C14 are independently selected from any stereoisomer orientation. For the pleuromutilin analog having the structure of formula 7-1, R₁ is selected from the group consisting of any one of the diverse pleuromutilin or pleuromutilin derivative side-chains as a C21 acyl side chain; R2 is selected from the group consisting of H, OH, N and O; R3 is selected from the group consisting of CH₂OH and CH₂X and X is selected from the group consisting of a halogen atom, a nitrogen atom, an alkyl group, an alkenyl group and an aryl group. For the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R₁ is selected from the group consisting of O, HN, HCH3, Net, NPr and NBu, and R₃ is selected from the group consisting alkylene groups.

In an aspect, the invention relates to a method of treating disease comprising administering a composition including a pleuromutilin analog or a pharmaceutical composition including a pleuromutilin analog to a patient in need thereof. The pleuromutilin analog has the structure of one of formulas 7-1 or 7-2:

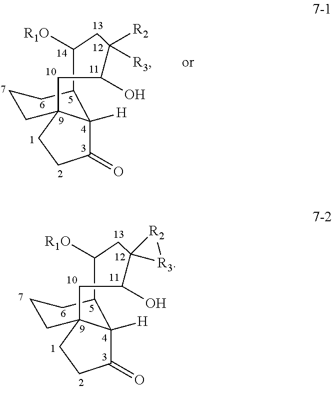

The bonds on C11, C12 or C14 are independently selected from any stereoisomer orientation. For the pleuromutilin analog having the structure of formula 7-1, R₁ is selected from the group consisting of any one of the diverse pleuromutilin or pleuromutilin derivative side-chains as a C21 acyl side chain; R₂ is selected from the group consisting of H, OH, N and O; R₃ is selected from the group consisting of CH₂OH and CH₂X and X is selected from the group consisting of a halogen atom, a nitrogen atom, an alkyl group, an alkenyl group and an aryl group. For the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R1 is selected from the group consisting of O, HN, HCH3, Net, NPr and NBu, and R3 is selected from the group consisting of alkylene groups.

In an aspect, the invention relates to a method of analyzing the affect of point mutations within a pleuromutilin compound. The method includes exposing a Mycobacterium tuberculosis model organism to a pleuromutilin analog. The pleuromutilin analog has the structure of one of formulas 7-1 or 7-2:

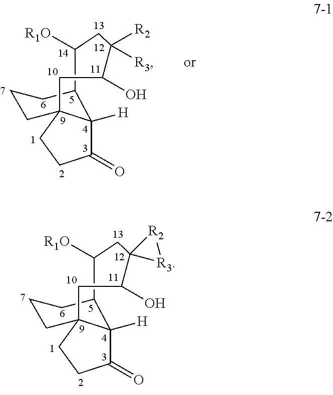

The bonds on C11, C12 or C14 are independently selected from any stereoisomer orientation. For the pleuromutilin analog having the structure of formula 7-1, R₁ is selected from the group consisting of any one of the diverse pleuromutilin or pleuromutilin derivative side-chains as a C21 acyl side chain; R₂ is selected from the group consisting of H, OH, N and O; R3 is selected from the group consisting of CH₂OH and CH₂X and X is selected from the group consisting of a halogen atom, a nitrogen atom, an alkyl group, an alkenyl group and an aryl group. For the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R₁ is selected from the group consisting of O, HN, HCH3, Net, NPr and NBu, and R3 is selected from the group consisting of alkylene groups.

In an aspect, the invention relates to a composition comprising a pleuromutilin skeleton having the structure of one of formulas 6-1, 6-2 or 6-3:

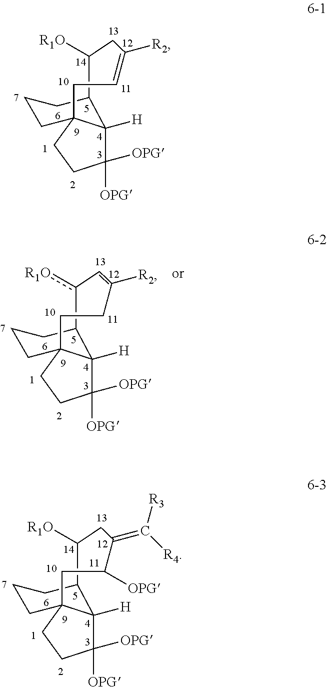

The C14-O bond in formulas 6-1, 6-2, and 6-3 and the C11-O bond in formula 6-3 are independently selected from any stereoisomer orientation. R₁ is selected from the group consisting of H, H and a hydroxyl protecting group, any one of the diverse pleuromutilin or pleuromutilin derivative side-chains, and any one of the diverse pleuromutilin or pleuromutilin derivative side-chains and a protecting group. PG is a hydroxyl protecting group. PG′ is a ketone protecting group. R2, R3 and R4 are independently selected from the group consisting of H, CH₃, an alkyl group, alkenyl group and an aryl group.

### DETAILED DESCRIPTION OF THE PREFERRED EMBODIMENTS

The words “a,” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

Embodiments herein provide chemistry to expand the family of compounds possessing the 8-6-5 tricyclic scaffold of the pleuromutilins and/or the polar functional groups (e.g., the C₃ keto, the C₁₁OH, and the C₁₄ ester side chain). Various positions on the scaffold and/or the polar functional groups are modified to provide the expanded family of compounds. The compounds provided herein are referred to as pleuromutilin analogs. Pleuromutilin analogs may be useful for biological activity. Embodiments herein also include methods of investigating the biological activity of pleuromutilin analogs produced by a method herein, the exploration of other methods of rapidly generating pleuromutilin-like structures, and the total synthesis of pleuromutilin itself.

An embodiment provides a method of synthesizing a pleuromutilin analog. The method includes providing a pleuromutilin skeleton. If it is not present on the skeleton, the method may include introducing any one of the diverse pleuromutilin or pleuromutilin derivative side-chains as a C21 acyl side chain, which is a modification on the oxygen bound to C14. C21 acyl side chain refers to the entire structure bound to the C14 carbonyl oxygen. The skilled artisan will recognize the set of diverse pleuromutilin or pleuromutilin derivative side-chains as a C21 acyl side chain. Examples of the diverse pleuromutilin or pleuromutilin derivative side-chains as a C21 acyl side chain in the art can be found in Davidovich et al., “Induced-fit tightens pleuromutilins binding to ribosomes and remote interactions enable their selectivity” (2007) Proc. Natl. Acad. Sci. Vol. 104(11): 4291-4296; Egger et al., “New Pleuromutilin Derivatives with Enhanced Antimicrobial Activity: I. Synthesis” (1976) The Journal of Antibiotics Vol. XXIX(9): 915-922; Egger et al., “New Pleuromutilin Derivatives with Enhanced Antimicrobial Activity: II. Structure-Activity Correlations” (1976) The Journal of Antibiotics Vol. XXIX(9): 923-927; Fischbach et al., “Antibiotics for Emerging Pathogens” (2009) Science Vol. 325: 1089-1093; Hunt, E., “Pleuromutilin antibiotics” (2000) Drugs of the Future Vol. 25(11): 1163-1168; Novak et al., “The pleuromutilin antibiotics: A new class for human use” (2010) Current Opinion in Investigational Drugs Vol. 11(2): 182-192; Phillips et al., “Pleuromutilin antibacterial agents: patent review 2002-2006” (2007) Expert Opinion Ther. Patents Vol. 17(4): 429-435; Riedl, K., “Studies on Pleuromutilin and Some of its Derivatives” (1976) The Journal of Antibiotics Vol. XXIX(2): 132-139; and Silver, L., “Challenges of Antibacterial Discovery” (2011) Clinical Microbiology Reviews Vol. 24(1): 71-109, which are incorporated herein by reference as if fully set forth. Examples of steps to introduce a pleuromutilin or pleuromutilin derivative side-chain as a C21 acyl side chain are provided herein, but the skilled artisan will recognize that additional acceptable routes of synthesis are possible for such a step. Any route of introduce a pleuromutilin or pleuromutilin derivative side-chain as a C21 acyl side chain may be utilized in embodiments herein.

The method of synthesizing a pleuromutilin analog also includes introducing one or more pleuromutilin analog substituent. As used herein, a pleuromutilin analog substituent is a modification at any position of the molecule relative to natural pleuromutilin. The modification may be at any position of the 8-6-5 tricyclic scaffold of the pleuromutilin and/or the polar functional groups (e.g., the C₃ keto, and the C₁₁OH). Embodiments include methods of synthesizing pleuromutilin itself or synthesizing pleuromutilin analogs that contain substituents at one or more specific position that are identical to the corresponding position in pleuromutilin. In these embodiments, term pleuromutilin analog substituent refers to substituents that are the same as or different than the substituents that form pleuromutilin itself.

The method of synthesizing a pleuromutilin analog may also include conducting two fold de-protection to form a C3 ketone and unveil a C11 hydroxyl. Two fold de-protection may include treatment with HCl and MeOH. The temperature during de-protection may be 0° C. Examples below provide specific steps of two fold de-protection. Modifications of the specific examples may be provided in embodiments herein where the modified de-protection also leads to formation of the C3 ketone and unveiling of the C11 hydroxyl.

The method may include providing a pleuromutilin skeleton having the structure of one of formulas 6-1, 6-2 or 6-3:

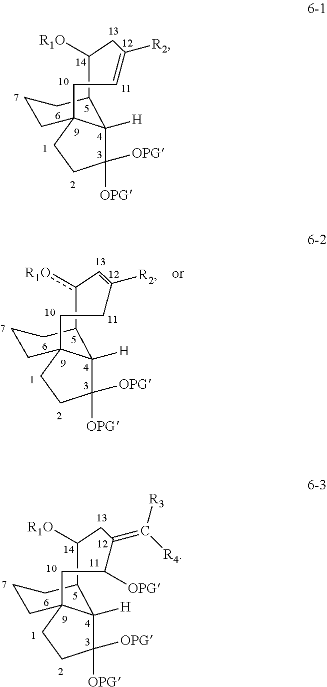

The C14-O bond in formulas 6-1, 6-2, and 6-3 and the C11-O bond in formula 6-3 are independently selected from any stereoisomer orientation. In formula 6-2, R₁ may not be present when a double bond is formed between C14 and the oxygen. R₁ may be selected from H, H and a hydroxyl protecting group, any one of the diverse pleuromutilin or pleuromutilin derivative side-chains, and any one of the diverse pleuromutilin or pleuromutilin derivative side-chains and a protecting group. PG may be a hydroxyl protecting group. PG′ may be a ketone protecting group. R2, R3 and R4 may be independently selected from the group consisting of H, CH₃, an alkyl group, alkenyl group and an aryl group. Examples of an alkyl group include but are not limited to ethyl, isopropyl, butyl, or pentyl. Examples of an akenyl group include but are not limited to vinyl, isopropenyl, propenyl, or butenyl. Examples of an aryl group include but are not limited to phenyl, furyl, or indolyl.

The pleuromutilin skeleton of formula 6-1 may be provided with variations. The variations may include different substituents or stereo configurations. A pleuromutilin skeleton of formula 6-1 may be provided having the structure of one of formulas 2-1a, 2-1aa, 2-1b, 2-1bb, 2-1, 4-1, or 4-1a:

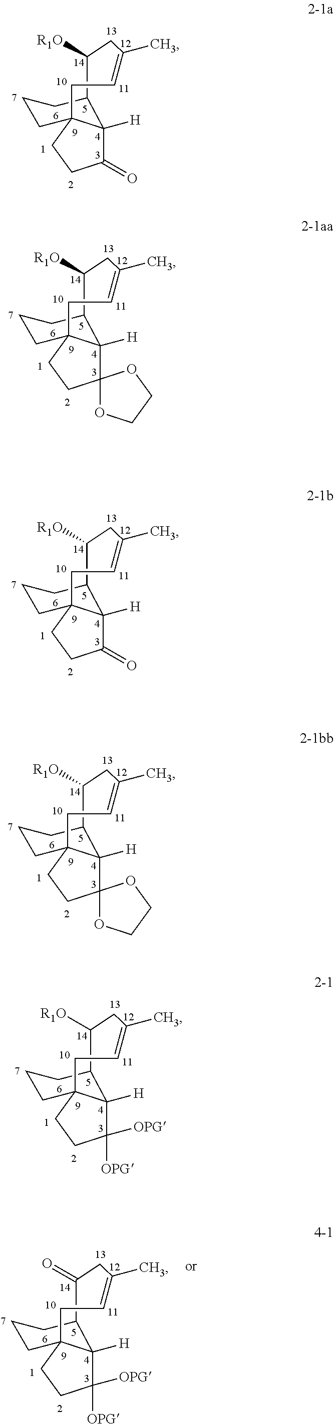

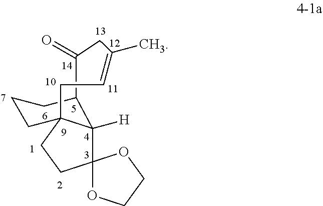

In formulas 2-1a, 2-1aa, 2-1b, 2-1bb, 2-1, 4-1, and 4-1a, R1 and OPG′ have the same meaning as provided above for formula 6-1.

The pleuromutilin skeleton of formula 6-2 may be provided with variations. The variations may include different substituents or stereo configurations. A pleuromutilin skeleton of formula 6-2 may be provided having the structure of one of formulas 3-1a, 3-1 or 5-1:

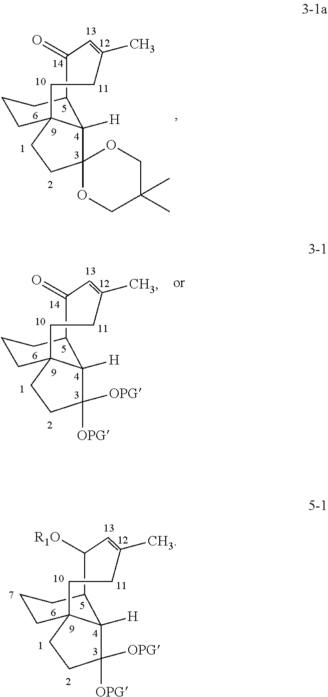

In formulas 3-1a, 3-1 and 5-1, OPG′ and R₁ have the same meaning as provided above for formula 6-2.

The pleuromutilin skeleton of formula 6-3 may be provided with variations. The variations may include different substituents or stereo configurations. A pleuromutilin skeleton of formula 6-2 may be provided having the structure of one of formulas 1-1a, 1-1b, 1-1c(1), 1-1c(2), or 1-1:

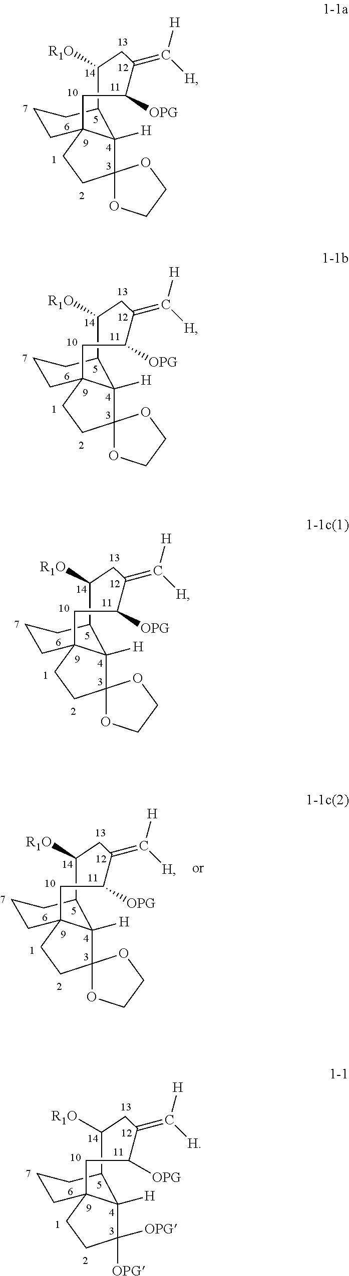

In formulas 1-1a, 1-1b, 1-1c(1), 1-1c(2), or 1-1, R1, OPG and OPG′ have the same meaning as provided above for formula 6-3.

As set for the above, the method of synthesizing a pleuromutilin analog may include structures having a hydroxyl protecting group, a PG that is a protecting group, and a PG′ that is a protecting group. The hydroxyl protecting group, PG and PG′ may be independently selected from any group that performs the function of being a protecting group during a synthesis scheme for the method of synthesizing a pleuromutilin analog. A hydroxyl protecting group may be selected from the group consisting of TBS (tert-butyldimethylsilyl), TES (triethylsilyl), TPS (tripropylsilyl) and MOM (methoxymethyl). A PG may be selected from the group consisting of the ketone TBS, TMS (trimethylsilyl), TES, TPS and MOM. A PG′ may be selected from the group consisting of the ketals in the skeletons of formula 1-1a and 3-1a.

Providing the pleuromutilin skeleton for the method of synthesizing a pleuromutilin analog may include making the pleuromutilin skeleton. A pleuromutilin skeleton may be arrived at by any suitable synthesis scheme from precursor compounds.

Making the pleuromutilin skeleton of formula 6-1 may be accomplished by a scheme including the steps a), b) and c) provided immediately below this paragraph. The skilled artisan will recognize conditions for carrying out the reactions in the following steps. Exemplary conditions are provided in the examples below. And any one set of conditions for carrying out the reactions may be provided in an embodiment herein.

Step a) includes a reaction step from precursor 1-1 to intermediate 1-1:

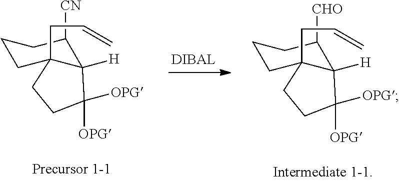

Step b) includes a reaction from intermediate 1 to intermediate 2:

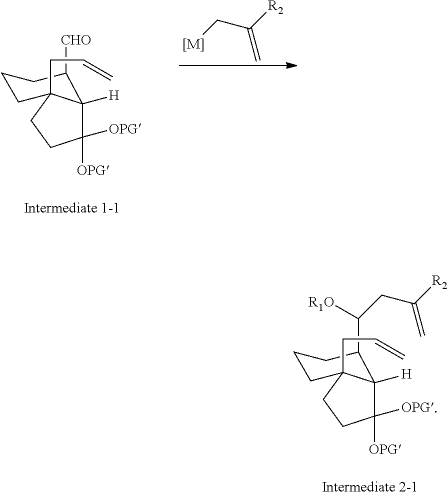

M is a metal agent. M may be selected from Zn, Mg, Li, Cr or Sm. Step c) includes a reaction from Intermediate 2 to a compound having the structure of formula 6-1:

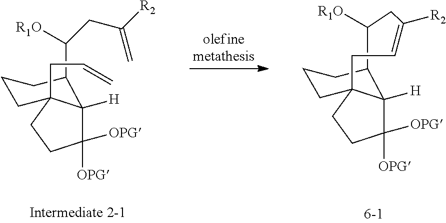

The specific examples below of making a compound having the structure of formula 6-1 may be adapted to form any compound having a structure of formula 6-1. Methods of making any one compound of formula 6-1 are provided herein. The method may include starting with precursor 1-1 or any intermediate along the way to the skeleton of formula 6-1. The method may include reactions to construct precursor 1-1. A method of making any one compound of formula 6-1 may be provided to create a compound of formula 6-1. A method of making any one compound of formula 6-1 may be implemented to provide a compound of formula 6-1 for a method of synthesizing a pleuromutilin analog herein.

Making the pleuromutilin skeleton of formula 6-2 may be accomplished by a scheme including the steps a), b) and c) provided immediately below this paragraph. The skilled artisan will recognize conditions for carrying out the reactions in the following steps. Exemplary conditions are provided in the examples below. And any one set of conditions for carrying out the reactions may be provided in an embodiment herein.

Step a) includes a reaction from precursor 1-2 to intermediate 1-2 by Suzuki coupling:

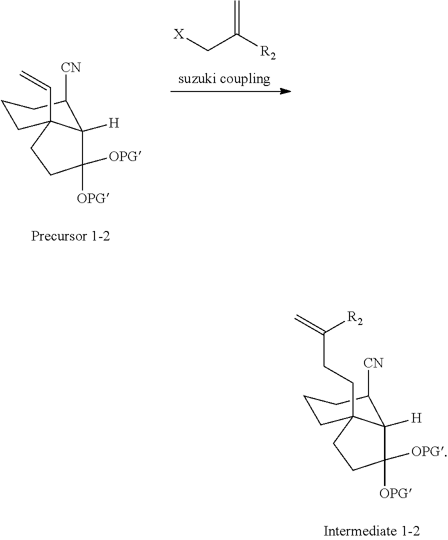

X may be a halogen. X may be Cl, Br or I.

Step b) includes sub-scheme bi) or sub-scheme bii). Sub-scheme bi) includes a reaction from intermediate 1-2 to intermediate 2-2:

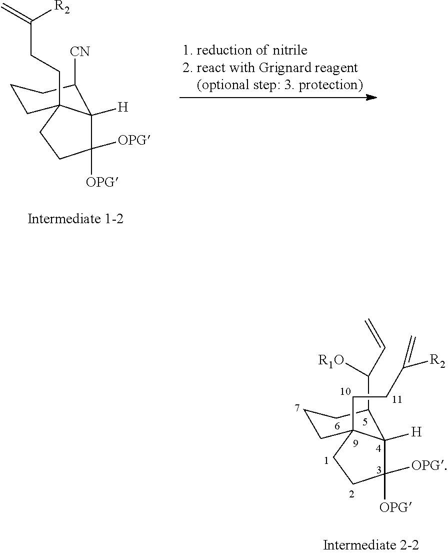

Sub-scheme bii) includes a reaction from intermediate 1-2 to 3-2:

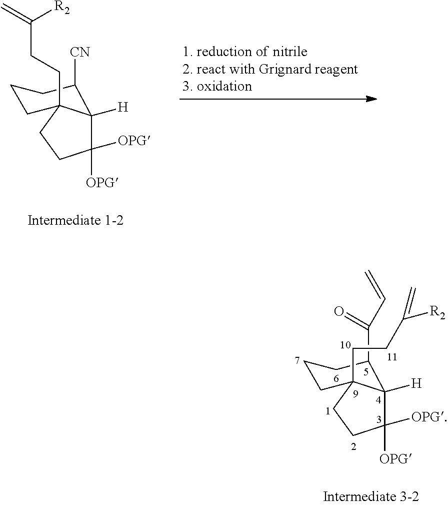

Step c) includes olefin metathesis to produce the pleuromutilin skeleton of formula 6-2a from intermediate 2-2 or the pleuromutilin skeleton of formula 6-2b form intermediate 3-2:

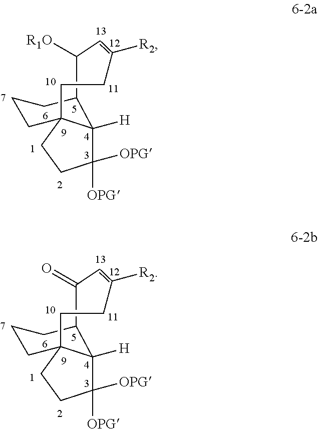

The specific examples below of making a compound having the structure of formula 6-2a or 6-2b may be adapted to form any compound having a structure of formula 6-2. Methods of making any one compound of formula 6-2 are provided herein. The method may include starting with precursor 1-2 or any intermediate along the way to the skeleton of formula 6-2. The method may include reactions to construct precursor 1-2. A method of making any one compound of formula 6-2 may be provided to create a compound of formula 6-2. A method of making any one compound of formula 6-1 may be implemented to provide a compound of formula 6-2 for a method of synthesizing a pleuromutilin analog herein.

Making the pleuromutilin skeleton of formula 6-3 may be accomplished by a scheme including the steps a), b) and c) provided immediately below this paragraph. The skilled artisan will recognize conditions for carrying out the reactions in the following steps. Exemplary conditions are provided in the examples below. And any one set of conditions for carrying out the reactions may be provided in an embodiment herein.

Step a) includes three substeps, listed below, to form intermediate 1-3 from precursor 1-3:

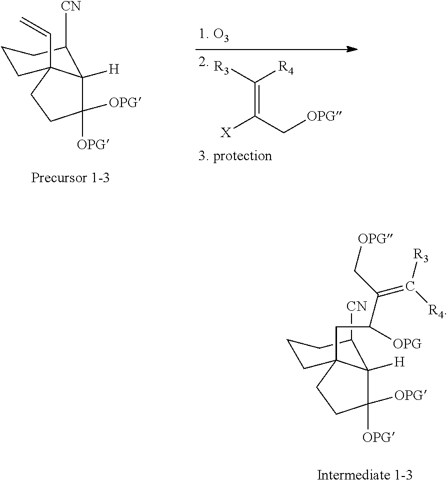

Step c) includes deprotection of intermediate 1-3 followed by reduction of nitrile to form intermediate 2-3:

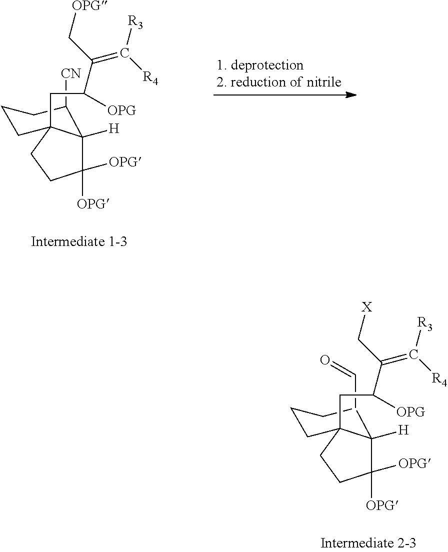

PG″ may be the same as PG, and X may be a halide. X may be Cl, Br or I.

Step c) includes a Nozaki-Hiyama-Kishi (NHK) reaction scheme using intermediate 2-3 to achieve the pleuromutilin skeleton having the structure of formula 6-3:

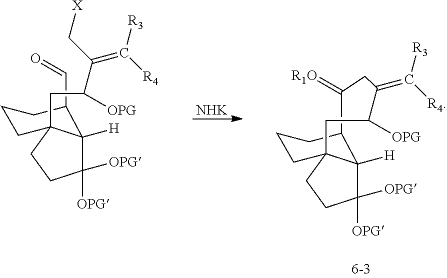

The specific examples below of making a compound having the structure of formula 6-3 may be adapted to form any compound having a structure of formula 6-3. The skilled artisan will readily understand adaptations to the specific examples below to provide any one compound having the structure of formula 6-3. Methods of making any one compound of formula 6-3 are provided herein. The method may include starting with precursor 1-3 or any intermediate along the way to the skeleton of formula 6-3. The method may include reactions to construct precursor 1-3. A method of making any one compound of formula 6-3 may be provided to create a compound of formula 6-3. A method of making any one compound of formula 6-3 may be implemented to provide a compound of formula 6-3 for a method of synthesizing a pleuromutilin analog herein.

Making a pleuromutilin skeleton may be accomplished by a scheme including the steps a) and b) provided immediately below this paragraph. The skilled artisan will recognize conditions for carrying out the reactions in the following steps. Exemplary conditions are provided in the examples below. And any one set of conditions for carrying out the reactions may be provided in an embodiment herein.

Step a) may include oxidation of precursor 1-4 to produce intermediate 1-4:

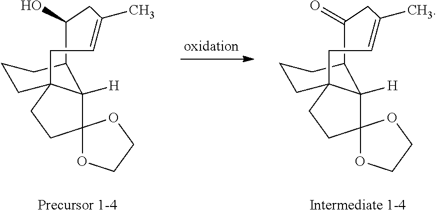

Step b) may include reduction of intermediate 1-4 to form skeletons having the structure of formulas 1-4a and 1-4-b

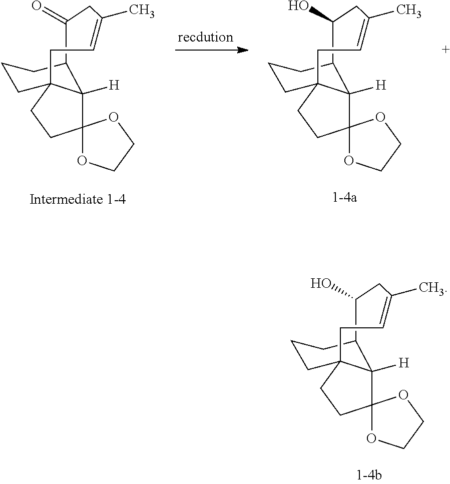

The specific examples below of making a compound having the structure of formula 6-3 may be adapted to form any compound having a structure of formula 6-3.

As set forth above, a method of synthesizing a pleuromutilin analog includes introducing one or more pleuromutilin analog substituent. A method of introducing one or more pleuromutilin analog substituent may be performed before, after or during a step of C14 acylation to introduce any one of the diverse pleuromutilin or pleuromutilin derivative side-chains as a C21 acyl side chain. Conducting two fold de-protection to form a C3 ketone and unveil the C11 hydroxyl may be performed at any point suitable for formation of the specific pleuromutilin analog. In an embodiment, two fold de-protection is performed after the steps of introducing one or more pleuromutilin analog substituent and the step of introducing any one of the diverse pleuromutilin or pleuromutilin derivative side-chains as a C21 acyl side chain.

A method of introducing one or more pleuromutilin analog substituent may include at least one step selected from dihyroxylation, epoxidation, hydroboration, ozonolysis, aziridination, difluorination, fluoride addition, epoxide opening, isomerization, or bromide addition, or alpha-difluorination on the C12 alkene.

A method of introducing one or more pleuromutilin analog substituent and introducing any one of the diverse pleuromutilin or pleuromutilin derivative side-chains by C14 hydroxyl acylation as a C21 acyl side chain may include one of the following schemes:

a) alkene epoxidation followed by C14 hydroxyl acylation;

b) alkene cyclopropanation followed by C14 hydroxyl acylation;

c) C14 hydroxyl acylation followed by alkene aziridination;

d) C14 hydroxyl acylation followed by singlet oxidation/Ph₃P;

e) alkene ozonolysis followed by Wittig reaction with R₁R₂C=PPh₃ and then C14 hydroxyl acylation;

f) alkene ozonolysis followed by C14 hydroxyl acylation;

g) C14 hydroxyl acylation followed by alkene hydroboration/oxidation;

h) alkene epoxidation followed by nucleophilic opening of epoxide then C14 hydroxyl acylation; or

i) C14 hydroxyl acylation followed by alkene dihydroxylation. The product(s) of one of schemes a-i, immediately above, may be subjected to two fold de-protection to form a C3 ketone and unveil the C11 hydroxyl. With a pleuromutilin skeleton having the structure of formula 6-3, where R₁, R₃ and R₄ are H and after two fold de-protection, scheme a) results in a compound having the structure of formula 51:

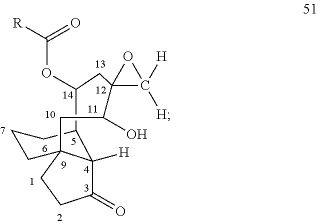

scheme b) results in a compound having the structure of formula 52:

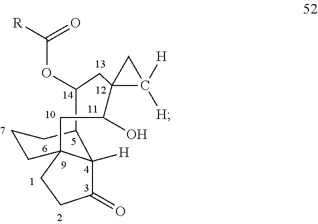

scheme c) results in a compound having the structure of formula 53:

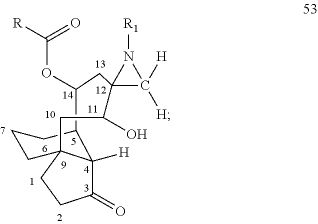

scheme d) results in a compound having the structure of formula 54:

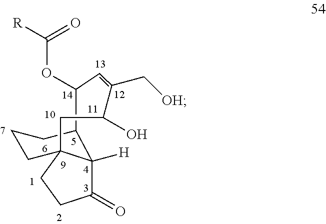

scheme e) results in a compound having the structure of formula 55:

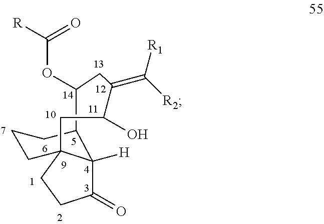

scheme f) results in a compound having the structure of formula 56:

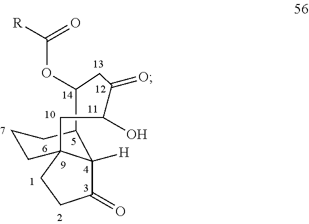

scheme g) results in a compound having the structure of formula 57:

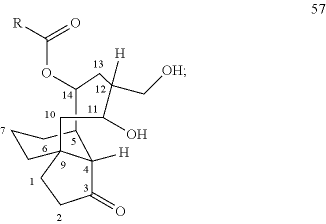

scheme h) results in a compound having the structure of formula 58:

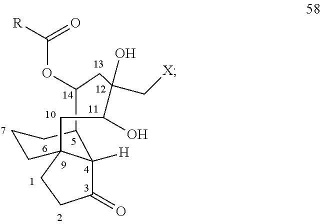

and
scheme i) results in a compound having the structure of formula 59:

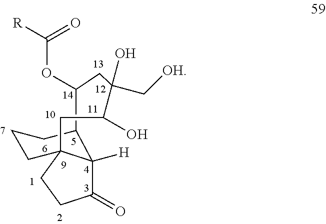

R as used in reference to formulas 52-59 may be any moiety. R as used in reference to formulas 52-59 may be a pleuromutilin side chain or any one of a pleuromutilin derivative side chain as a C21 acyl side chain. R₁ as used in reference to formula 53 may be any moiety, or may be one of the R₃ as listed for formula 6-2 above. R₁ as used in reference to formula 55 may be any moiety, or may be one of the R₃ as listed for formula 6-2 above. R₂ as used in reference to formula 55 may be any moiety, or may be one of the R4 as listed for formula 6-2 above. X as used in this paragraph may be a nitrogen atom, a fluorine atom, an alkyl group, an alkenyl group or an aryl group. Examples of an alkyl group include but are not limited to ethyl, isopropyl, butyl, or pentyl. Examples of an akenyl group include but are not limited to vinyl, isopropenyl, propenyl, or butenyl. Examples of an aryl group include but are not limited to phenyl, furyl, or indolyl.

In an embodiment a composition including pleuromutilin analog having a structure of one formulas 7-1 or 7-2 is provided:

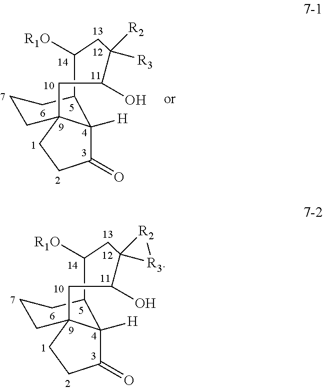

The bonds on C11, C12 or C14 are independently selected from any stereoisomer orientation. The composition may include or consist of a pleuromutilin analog or collection of pleuromutilin analogs. The composition may include pleuromutilin analog or collection of pleuromutilin analogs in combination with other substances.

In embodiment, a pleuromutilin analog has the structure of formula 7-1 and R₁ may be any one of the diverse pleuromutilin or pleuromutilin derivative side-chains as a C21 acyl side chain, R₂ may be H, OH, N or O, R3 may be CH₂OH or CH₂X and X may be a halogen atom, a nitrogen atom, an alkyl group, an alkenyl group or an aryl group. Examples of an alkyl group include but are not limited to methyl, ethyl, or propyl. Examples of an akenyl group include but are not limited to vinyl, propenyl, or isopropenyl. Examples of an aryl group include but are not limited to phenyl, furyl, or indolyl. The halogen atom may be any halogen atom. The halogen atom may be a fluorine, chlorine, bromine or iodine.

In an embodiment, a pleuromutilin analog has the structure of formula 7-2 and R₁ may be any one of the diverse pleuromutilin or pleuromutilin derivative side-chains as a C21 acyl side chain, and R₂ and R₃ may be alkylene groups. The alkylene group for one or both of R₂ or R₃ may be CH₂. The alkylene group may be methyl, ethyl isopropyl or butyl.

In an embodiment, a pleuromutilin analog has the structure of formula 7-2 and R1 may be O, HN, HCH3, Net, NPr or NBu, and R3 may be CH₂ or other alkylene group. The alkylene group may be methyl, ethyl isopropyl or butyl.

In an embodiment, any pleuromutilin analog herein has the pleuromutilin or pleuromutilin derivative C21 acyl side chain that is a tiamulin C21 acyl side chain, a pleuromutilin C21 acyl side chain or a retapamulin C21 acyl side chain.

In an embodiment a pleuromutilin analog, the C3 and C21 carbonyl and the C11 hydroxyl groups of a pleuromutilin scaffold are constant in the analog, but substituents at other positions are varied. In some embodiments, all substituents may be varied, including the C3, C21 and C11 positions.

In an embodiment, a pleuromutilin analog may be provided having the structure of any of formulas II, III, IV or V:

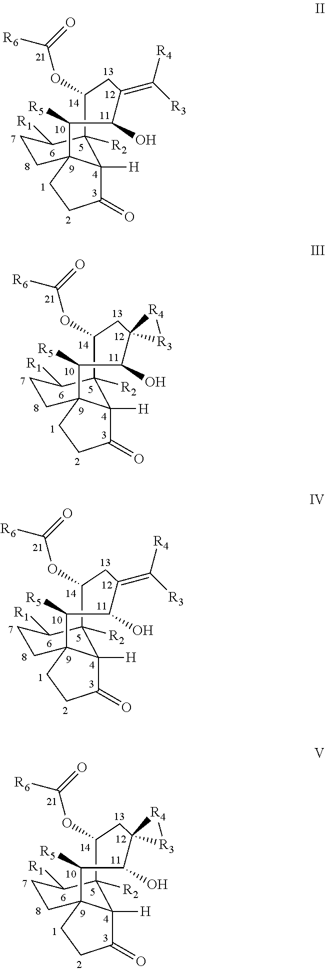

R₁-R₅ for any one of formulas II, III, IV and V may be independently selected from hydrogen, fluorine or alkyl side chains having one to five carbon atoms. R6 may be selected from alkyl, aryl and heteroatoms. R6 may have more than five carbons. As shown, a pleuromutilin scaffold is common to structures II and IV and III and V. R1, R2, R3, R4, R5 and R6 may be selected from any moiety, wherein the combination of R1, R2, R3, R4, R5 and R6 provides a biologically active compound. Unexpectedly, hydroxyl at position 11 may be provided in either configuration represented in structures II, III, IV and V.

A pleuromutilin analog may be provided having the structure of any one of SDL-267-1, SDL-267-2, SDL-267-3, SDL-267-4, SDL-267-5, SDL-267-6, SDL-267-7, SDL-267-8, SDL-267-9, SDL-267-10, SDL-267-11, SDL-267-12, SDL-267-13 or SDL-267-14. SDL-267-1:

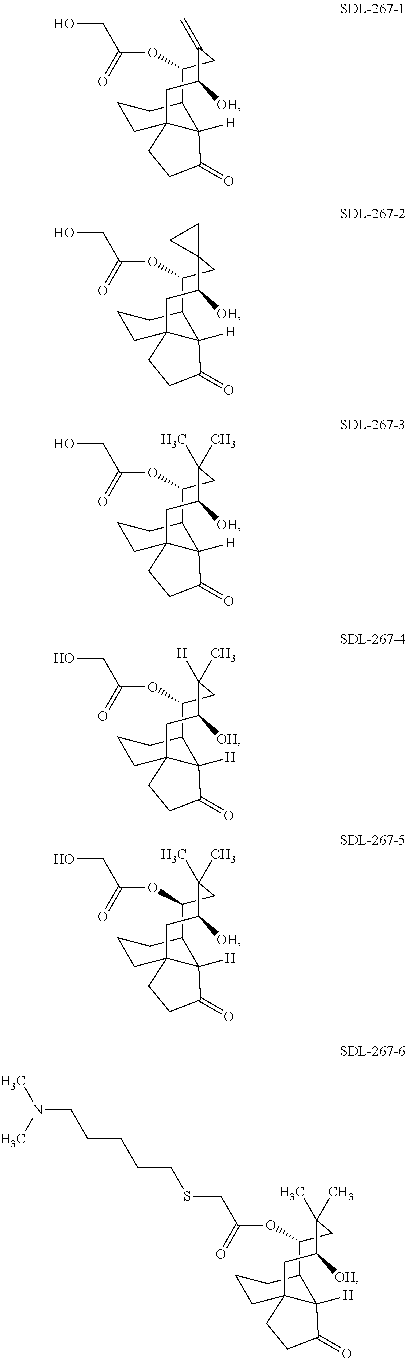

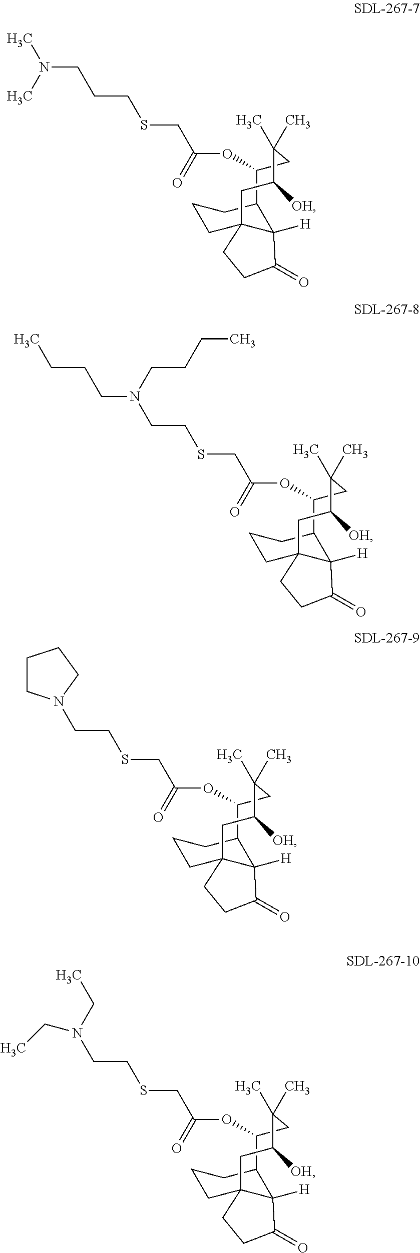

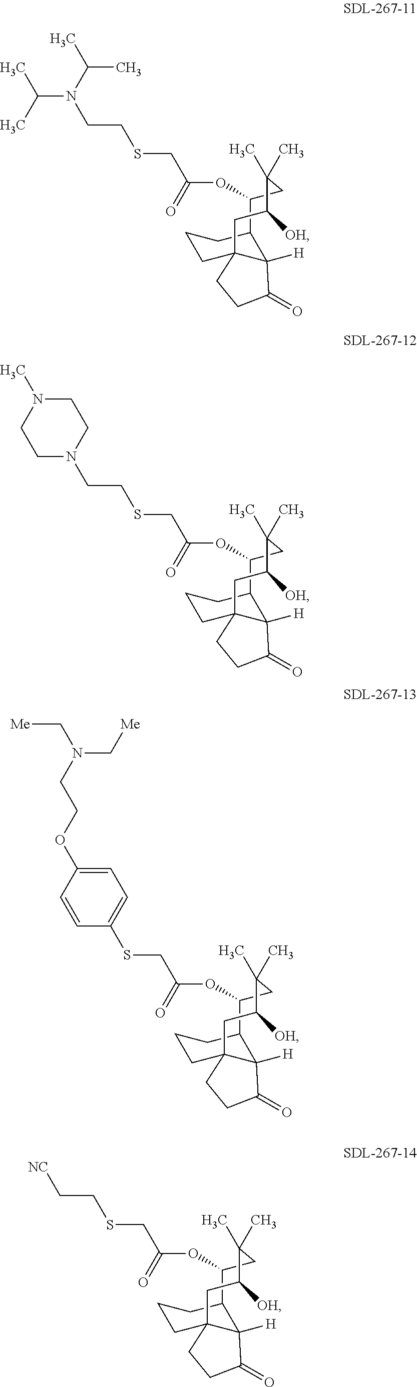

In an embodiment the scaffold of pleuromutilin, retapamulin, tiamulin or any analog herein may be provided as a scaffold for further modification to produce a further pleuromutilin analog. The scaffolds may be modified through semi-synthesis. Alternatively, the scaffolds may be produced as described above or in the examples but with modifications built in to the synthesis scheme (mutation through synthesis) so that the final product includes the modification. The modifications may be site-selective “point mutations.” In another alternative, an analog may be arrived at through a combination of semi-synthesis and mutation through synthesis.

A pleuromutilin analog may be provided having a structure that is a hybrid of any of the pleuromutilin analog structures herein.

In an embodiment, a pharmaceutical composition comprising a pleuromutilin analog or a pharmaceutically acceptable salt or solvate thereof is provided. Any one or more pleuromutilin analog herein or a pharmaceutically acceptable salt or solvate thereof herein may be provided in a pharmaceutical composition herein. A pleuromutilin analog in a pharmaceutical composition may have the structure of one of formulas 7-1 or 7-2:

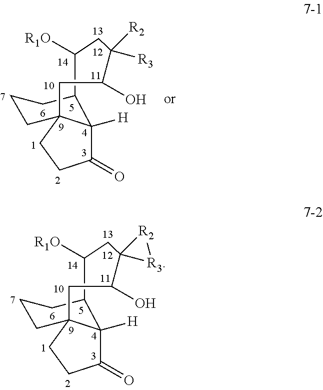

The bonds on C11, C12 or C14 are independently selected from any stereoisomer orientation. The pharmaceutical composition may include or consist of a pleuromutilin analog or collection of pleuromutilin analogs or pharmaceutically acceptable salts thereof. The pharmaceutical composition may include a pleuromutilin analog or collection of pleuromutilin analogs in combination with one or more other substances.

Any pharmaceutically acceptable salt or solvate of any one or more pleuromutilin analog or skeleton herein may be provided in a pharmaceutical composition herein. Pharmaceutically acceptable salts that may be included in embodiments herein can be found in Handbook of Pharmaceutical Salts: Properties, Selection, and Use, Stahl and Wermuth (Eds.), VHCA, Verlag Helvetica Chimica Acta (Zurich, Switzerland) and WILEY-VCH (Weinheim, Federal Republic of Germany); ISBN: 3-906390-26-8, which is incorporated herein by reference as if fully set forth. The pharmaceutical composition herein may be provided with a pharmaceutically acceptable carrier, which may be selected from but is not limited to one or more in the following list: ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts or electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.) and phosphate buffered saline (PBS).

Examples of formulations including pleuromutilin derivatives and methods of using the same as antimicrobials can be found in WO 2009/009812, which is incorporated herein by reference as if fully set forth. Any one or more pleuromutilin skeleton or analog herein may replace the pleuromutilin derivatives of WO 2009/009812 in formulations or methods described in WO 2009/009812. Such a formulation may be provided as a composition or a pharmaceutical composition herein.

In embodiment, a pleuromutilin analog in a pharmaceutical composition has the structure of formula 7-1 or a pharmaceutically acceptable salt or solvate thereof, and R₁ may be any one of the diverse pleuromutilin or pleuromutilin derivative side-chains as a C21 acyl side chain, R₂ may be H, OH, N or O, R3 may be CH₂OH or CH₂X and X may be a halogen atom, a nitrogen atom, an alkyl group, an alkenyl group or an aryl group. Examples of an alkyl group include but are not limited to methyl, ethyl, or propyl. Examples of an alkenyl group include but are not limited to vinyl, propenyl, butenyl, or isopropenyl. Examples of an aryl group include but are not limited to phenyl, furyl, or indolyl. The halogen atom may be any halogen atom. The halogen atom may be a fluorine, chlorine, bromine or iodine.

In an embodiment, a pleuromutilin analog in a pharmaceutical composition has the structure of formula 7-2 or a pharmaceutically acceptable salt or solvate thereof, and R₁ may be any one of the diverse pleuromutilin or pleuromutilin derivative side-chains as a C21 acyl side chain, and R₂ and R₃ may be alkylene groups. The alkylene group for one or both of R₂ or R₃ may be CH₂, or other alkylene group including but not limited to methyl, ethyl, isopropyl, or butyl.

In an embodiment, a pleuromutilin analog in a pharmaceutical composition has the structure of formula 7-2 or a pharmaceutically acceptable salt or solvate thereof, and R1 may be O, HN, HCH3, Net, NPr or NBu, and R3 may be CH₂ or other alkylene group including but not limited to methyl, ethyl, isopropyl, or butyl.

In an embodiment, any pleuromutilin analog or pharmaceutically acceptable salt or solvate thereof in a pharmaceutical composition herein has the pleuromutilin or pleuromutilin derivative side-chain that is a tiamulin side chain, a pleuromutilin side chain or a retapamulin side chain.

An embodiment provides a method of treating disease comprising administering a pharmaceutical composition including a pleuromutilin analog or a pharmaceutically acceptable salt or solvate thereof to a patient in need thereof. Any one or more pleuromutilin analog herein or a pharmaceutically acceptable salt or solvate thereof herein may be provided in a pharmaceutical composition for a method of treating disease herein. The pharmaceutical composition may be a pharmaceutical composition as described above. The patient in need thereof may have a microbial infection, an infection by bacteria, an infection by gram negative bacteria, an infection by Staphylococcus, an infection by Staphylococcus aureous, an infection by Staphylococcus pyogenes, an infection by Mycobacterium, an infection by Mycobacterium tuberculosis, tuberculosis, a skin infection or a lung infection. The method may be implemented to treat a microbial infection, an infection by bacteria, an infection by gram negative bacteria, an infection by Staphylococcus, an infection by Staphylococcus aureous, an infection by Staphylococcus pyogenes, an infection by Mycobacterium, an infection by Mycobacterium tuberculosis, tuberculosis, a skin infection or a lung infection. The pleuromutilin analog or a pharmaceutically acceptable salt or solvate thereof may be administered at any effective concentration, where effectiveness is measured by an improvement of a patient compared to an untreated patient. The pleuromutilin analog or a pharmaceutically acceptable salt or solvate thereof may be administered at a concentration of 1 μg/ml to 1 mg/ml, 900 μg/ml, 800 μg/ml, 700 μg/ml, 600 μg/ml, 500 μg/ml, 400 μg/ml, 300 μg/ml, 200 μg/ml, 100 μg/ml, 95 μg/ml, 90 μg/ml, 85 μg/ml, 80 μg/ml, 75 μg/ml, 70 μg/ml, 65 μg/ml, 60 μg/ml 55 μg/ml, 50 μg/ml, 45 μg/ml, 40 μg/ml, 35 μg/ml, 30 μg/ml, 25 μg/ml, 20 μg/ml, 15 μg/ml, 10 μg/ml, 5 μg/ml and 1 μg/ml. In an embodiment, the pleuromutilin scaffolds or analogs are administered at a concentration of 25 μg/ml, 50 μg/ml, 100 μg/ml, or 200 μg/ml. The dose delivered may be in a range of 1 μg/ml-200 μg/ml.

An embodiment provides a method of analyzing the affect of point mutations within a pleuromutilin compound. The method may include exposing a Mycobacterium tuberculosis model organism to any composition having a pleuromutilin analog herein. The affects of the pleuromutilin analog on the Mycobacterium tuberculosis model organism may be assessed in the method. The method may include serial or parallel experiments exposing a Mycobacterium tuberculosis model organism to any composition having a pleuromutilin analog herein, where different pleuromutilin analogs or different concentrations of a pleuromutilin analog are tested in the different experiments. The minimal inhibitory concentration of a pleuromutilin analog may be determined in the step of assessing.

The method of analyzing the affect of point mutations within a pleuromutilin compound that may include exposing a bacterial ribosome to any composition having a pleuromutilin analog herein. The affects of the pleuromutilin analog on the ribosome may be assessed by any known method of probing ribosome-ligand interactions. The method may include serial or parallel experiments exposing a bacterial ribosome to any composition having a pleuromutilin analog herein, where different pleuromutilin analogs or different concentrations of a pleuromutilin analog are tested in different experiments.

In an embodiment, a composition is provided that includes at least one pleuromutilin skeleton having the structure of one of formulas 6-1, 6-2 or 6-3:

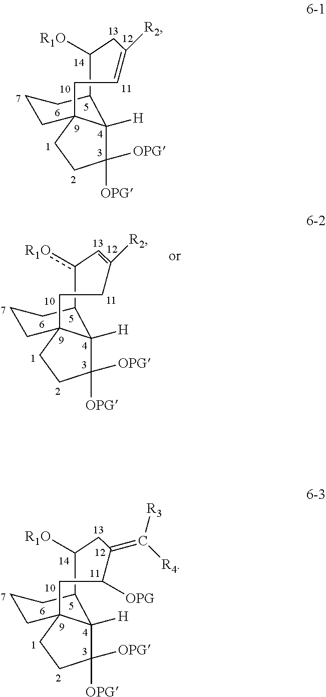

The C14-O bond in formulas 6-1, 6-2, and 6-3 and the C11-O bond in formula 6-3 may be independently selected from any stereoisomer orientation. In formula 6-2, R₁ may not be present if a double bond exists between C14 and the oxygen. R₁ may be H, H and a hydroxyl protecting group, any one of the diverse pleuromutilin or pleuromutilin derivative side-chains, or any one of the diverse pleuromutilin or pleuromutilin derivative side-chains and a protecting group. PG may be a hydroxyl protecting group. PG′ may be a ketone protecting group. R2, R3 and R4 may be independently selected from H, CH₃, an alkyl group, alkenyl group or an aryl group. Examples of an alkyl group include but are not limited to methyl, ethyl, or propyl. Examples of an alkenyl group including but not are limited to vinyl, propenyl, butenyl, or isopropenyl. Examples of an aryl group include but are not limited to phenyl, furyl, or indolyl.

The composition including at least one pleuromutilin skeleton may have a pleuromutilin skeleton having the structure of one of formulas 2-1a, 2-1aa, 2-1b, 2-1bb, 2-1, 4-1, 4-1a, 3-1a, 3-1, 5-1, 1-1a, 1-1b, 1-1c(1), 1-1c(2), or 1-1:

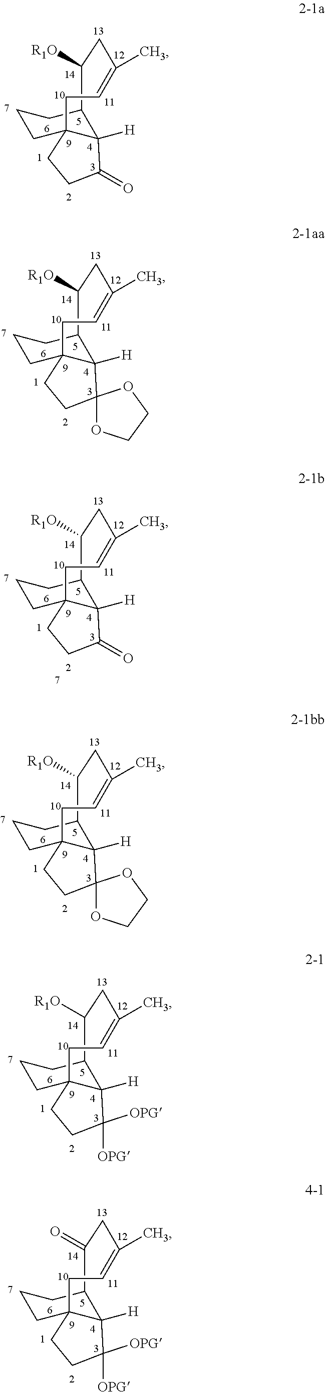

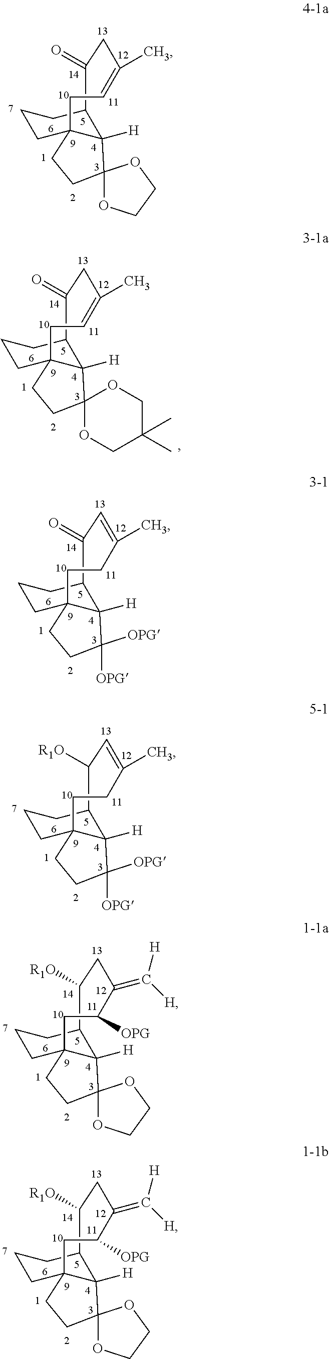

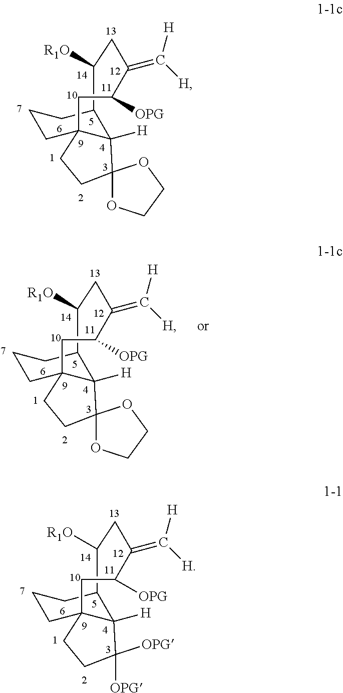

Additional embodiments herein include those formed by supplementing any one embodiment with one or more element from any one or more other embodiment herein.

Examples—The following non-limiting examples are provided to illustrate particular embodiments. The embodiments throughout may be supplemented with one or more detail from any one or more example below.

### SUMMARY OF SELECTED EXAMPLES

Two examples include distinct and direct routes to syntheses of the pleuromutilin ring system, including a “tiamulin-like” analogue that displayed significant activity against a tuberculosis model organism. One utilizes a ring closing metathesis reaction scheme, and the other utilizes an NHK reaction scheme. Both of these routes incorporate syntheses of a 5-6 bicyclic core from simple, widely available enones and accomplish the formation of substrates suitable for a final closure of the 8-membered ring. The resulting pleuromutilin skeletons are adaptable for pleuromutilin analog synthesis.

### Example 1

The first route utilizes an intramolecular ring-closing metathesis (RCM) reaction to establish the 8-membered ring:

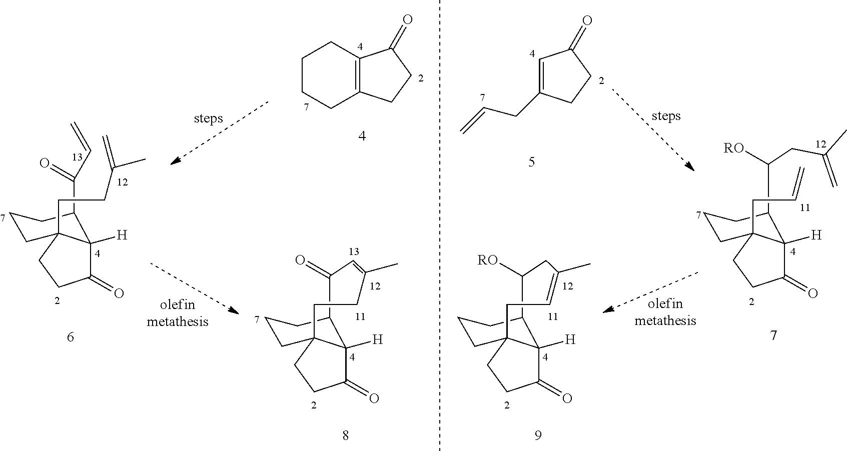

RCM has emerged as a powerful tool for constructing medium and large rings; and the olefin that is left in its wake can be used for subsequent derivatization to make a pleuromutilin analog. For a review on the RCM reaction see: Furstner, A. “Olefin Metathesis and Beyond” Angew. Chem. Int. Ed. 2000, 39, 3012-3043, which is incorporated herein by reference as if fully set forth. Starting from enones 4 and 5, metathesis precursors 6 and 7 were obtained via short sequences. RCM provides the desired 8-6-5 tricycles 8 and 9 in 10 and 7 steps respectively. Subsequent modification of the resultant core may involve oxidation state and stereochemical manipulation due to geometrical constraints of RCM. Nevertheless, this strategy provides access to a variety of novel structures with features of pleuromutilin.

Compounds 6 and 7 were considered potential substrates for ring closure between C12 and C13, or C11 and C12 respectively. This approach provides the opportunity to further manipulate the product olefins (8 and 9) in the creation of a diverse set of pleuromutilin analogs. Construction of the 8-6-5-tricyclic skeleton began with the 1,4-addition of an alkyl cuprate to known enone 4:

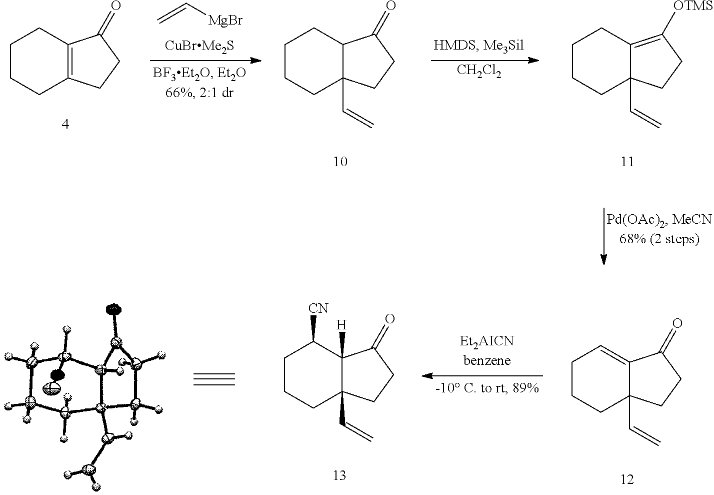

Treatment of enone 4 with vinylmagnesium bromide in the presence of a copper (I) salt and boron trifluoride etherate gives rise to ketone 10 in good yield as a 2:1 mixture of diastereomers. Exposure of ketone 10 to trimethylsilyl iodide and hexamethyldisilazane (HMDS) generated the thermodynamically favored tetrasubstituted silyl enol ether 11 exclusively, which upon Saegusa oxidation provided enone 12. Addition of diethylaluminum cyanide to enone 12 yielded cis-fused bicycle 13 in 89% yield as a single diastereomer. The structure of 13 was confirmed by X-ray crystallography.

Ketone 13 was protected as the corresponding ketal 14:

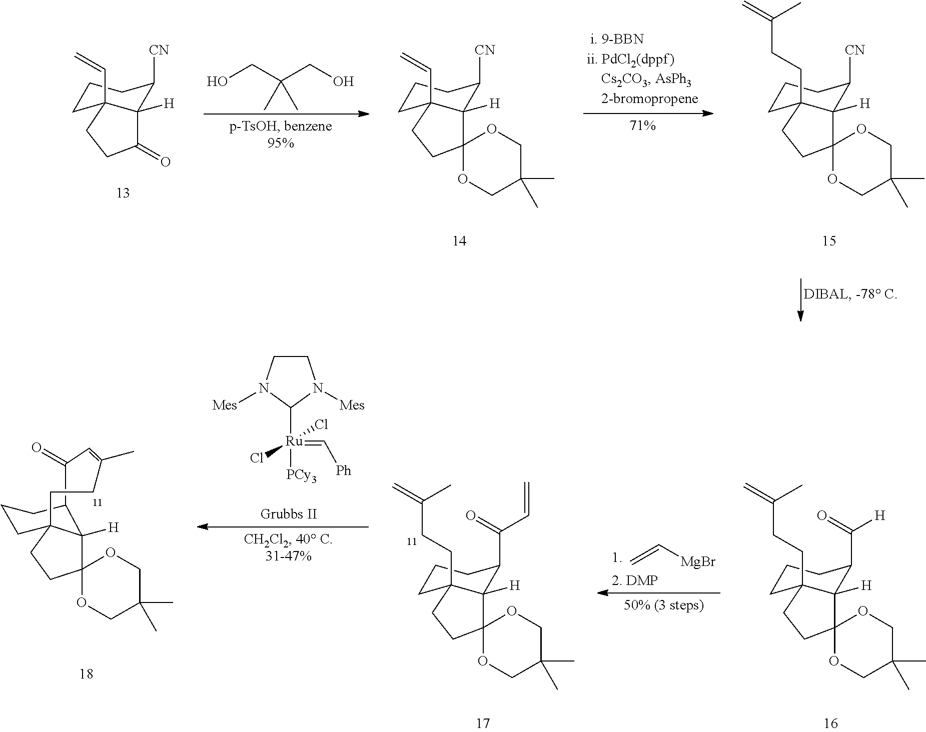

Compound 14 was then subjected to hydroboration with 9-BBN to generate an intermediate alkylborane. Subsequent Suzuki coupling with 2-bromo-propene afforded compound 15 in about 70% yield. Nitrile 15 was relatively unreactive towards the direct addition of vinyl Grignard reagents. Therefore, a three-step procedure was performed, which involved (1) reduction of 15 with diisobutylaluminum hydride (DIBAL) to give 16, (2) Grignard addition, and (3) oxidation of this secondary alcohol with Dess-Martin periodinane (DMP). By this sequence, key precursor 17 was produced in 50% yield over the three steps, requiring only one purification by column chromatography. Heating compound 17 at reflux with Grubbs second-generation olefin metathesis catalyst in dichloromethane gave rise to the desired product 18 containing the unique 8-6-5 tricyclic skeleton of pleuromutilin. Related compounds containing silyloxy groups at C11 did not undergo intramolecular olefin metathesis.

In order to incorporate functional groups at the C11 position of the pleuromutilin carbon scaffolds, an alternative metathesis route was devised. A cuprate derived from Grignard reagent 19 was added to known enone 5 in the presence of chlorotrimethylsilane:

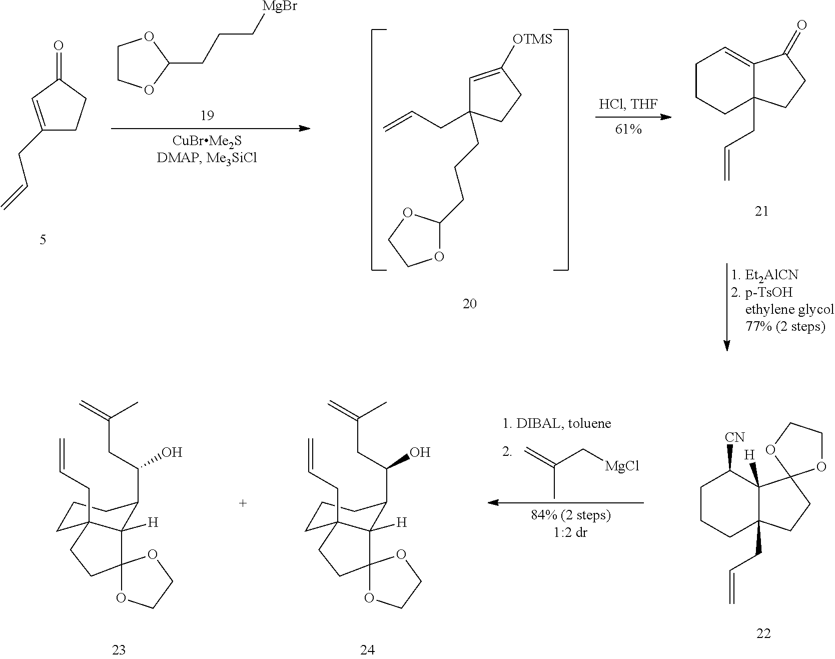

A crude solution containing silyl enol ether 20 was heated to reflux with aqueous hydrochloric acid, giving bicycle 21 via a deprotection/intramolecular aldol condensation sequence. The diastereoselective conjugate addition of cyanide to enone 21 and ketalization then provided nitrile 22 over the two steps. Reduction of nitrile 22 with DIBAL gave the corresponding aldehyde, which was treated with methallylmagnesium chloride to afford alcohols 23 and 24 in 84% yield as a 1:2 mixture of diastereomers that could be separated by column chromatography.

Alcohol 24 was converted to its epimer 23 via a two step sequence involving oxidation with DMP and selective reduction with lithium aluminum hydride:

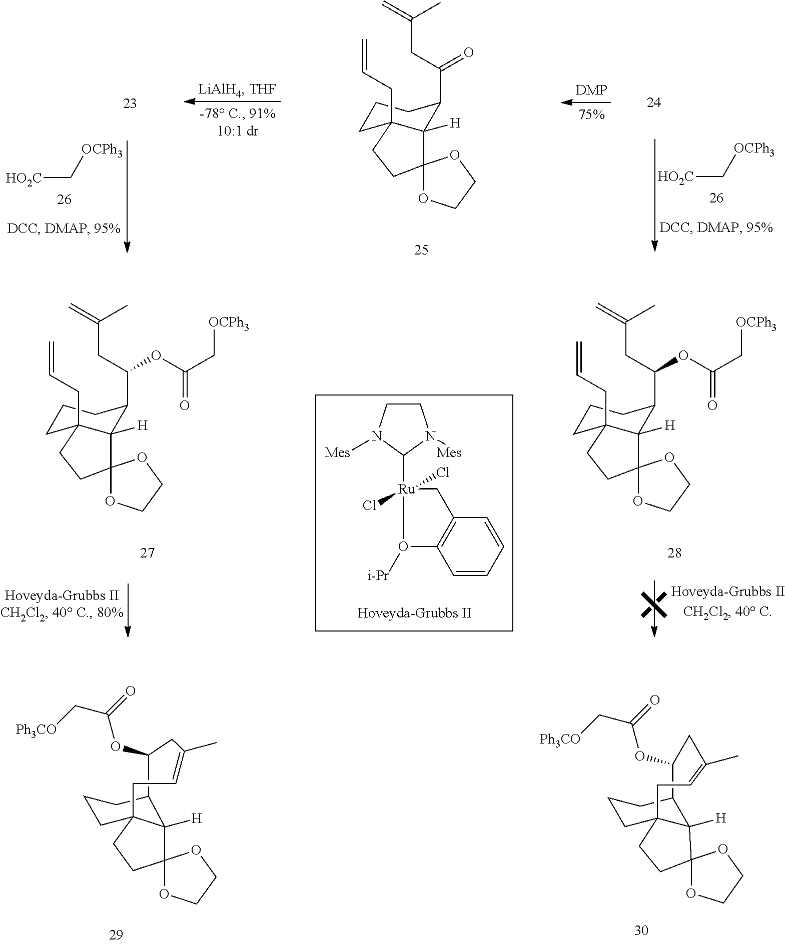

Alcohols 23 and 24 were independently coupled with acid 26 using N,N′-dicyclohexylcarbodiimide (DCC) to give esters 27 and 28, respectively. Heating ester 27 at reflux with the Hoveyda-Grubbs second-generation olefin metathesis catalyst gave rise to a pleuromutilin-like tricycle 29 in 80% yield. Under dilute conditions, ester 28 underwent only slow dimerization.

Treatment of metathesis product 29 with 3-chloroperoxy-benzoic acid (m CPBA) yielded epoxide 31 as a single diastereomer:

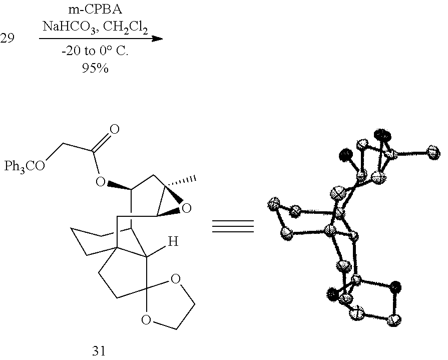

X-ray analysis of epoxide 31 allowed the unambiguous assignment of stereochemistry in 29 and related compounds. Although the relative configuration of C11 in the synthetic constructs does not match that of pleuromutilin itself, the epimeric series could still be effective, or be used to generate a series of derivatives and contribute to structure-activity relationship (SAR) studies.

A variety of related derivatives were prepared from compounds produced:

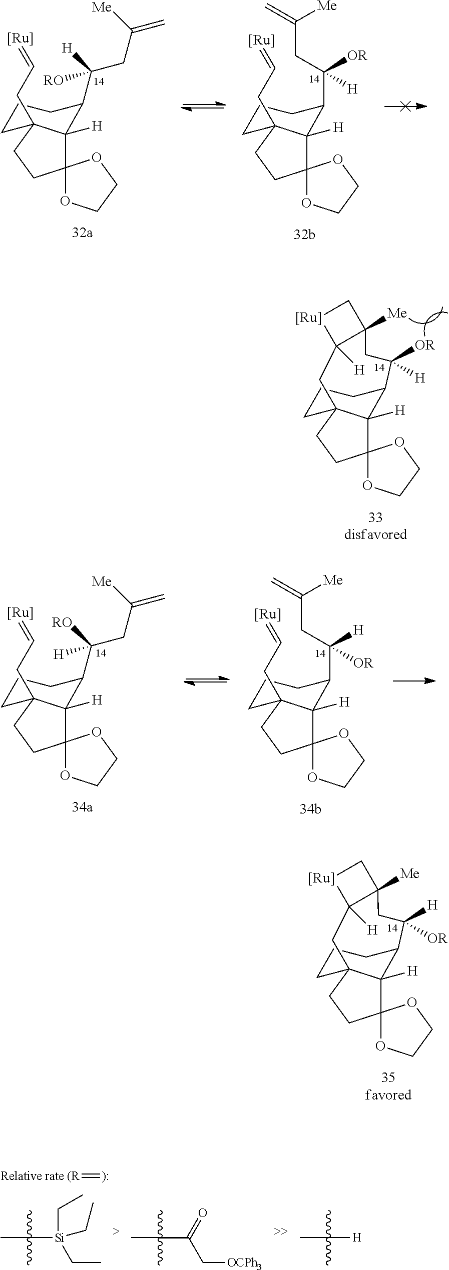

When these compounds were subjected to the conditions for olefin metathesis, the results mirrored observations regarding esters 27 and 28: the viability of the metathesis process was determined by the configuration at C14. Possibly, the diaxial interaction between the hydroxy-derived group of C14 and the methyl group on C12 prevents the formation of metallocyclobutane 33, while this steric repulsion is not present in epimer 35. A marked rate enhancement was observed in the ring-closing metathesis reactions of derivatives with bulky groups at C14. This could be interpreted as an alteration in the conformational bias of the molecule toward 33b, where the pendent olefins should be more ideally suited to engage the metathesis catalyst.

Only slight modifications of the route above would be required to incorporate other means of functionalization.

### Example 2

The second route involves a sequential utilization of the Nozaki-Hiyama-Kishi (NHK) reaction to fashion the 8-membered ring of a more advanced pleuromutilin scaffold. A first phase included intermolecular NHK coupling; for example:

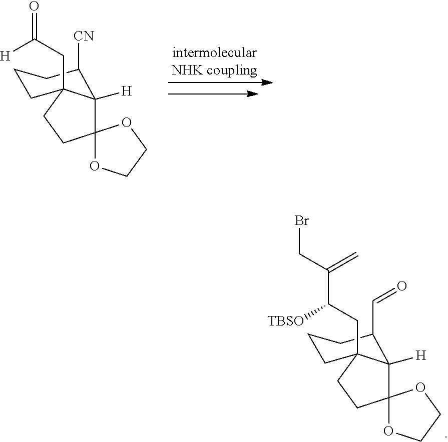

And the second phase included intramolecular NHK cyclization. An example of intramolecular NHK cyclization from the product directly above includes forming the following product:

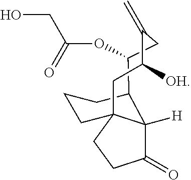

The first phase and second phase examples directly above are not limiting.

Like RCM, the NHK process is also capable of forming medium-sized rings in an efficient manner.

Generally, a compound for entering NHK can be used in an intermolecular NHK reaction that affords a 1:1 epimeric mixture of products after a nitrile reduction and bromide formation. The resultant aldehyde can then participate in an intramolecular, diastereoselective NHK reaction. Attachment of a glycolic acid side-chain provides a 8-6-5 pleuromutilin scaffold. The NHK linchpin strategy was used to install a three carbon unit onto a 1,3-diaxial dialdehyde or dialdehyde surrogate:

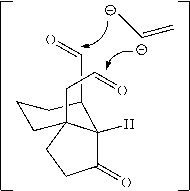

NHK cyclizations have a propensity to form medium sized rings, and the approach provided the propellane structure of type 4, bearing an exocyclic olefin.

The synthesis began from known cyclopentenone 6:

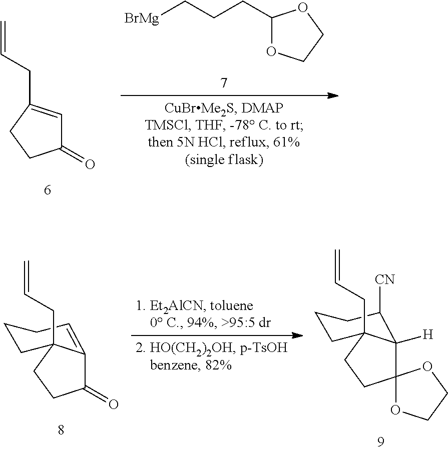

Conjugate addition of the higher-order cuprate derived from known Grignard reagent 7 gave a silyl enol ether, which upon acid quench induced a cyclocondensation event to afford allylic substituted hydrindenone system 8 in a single flask. Compound 9 was achieved by the reaction scheme shown above.

The reaction scheme below including ozonolysis with compound 9 provided cyano aldehyde 10 in high yield (2 steps):

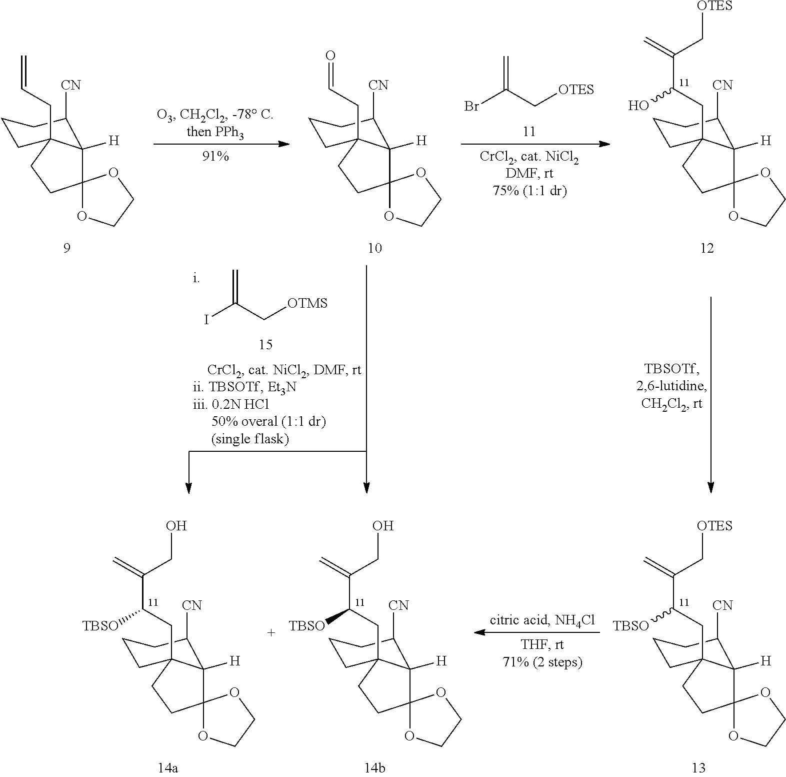

Exposure of aldehyde 10 to vinyl bromide 11 in the presence of CrCl₂ and catalytic NiCl₂ afforded the desired 1,1-disubstituted allylic alcohol 12. Protection of the resultant alcohol as the tert-butyldimethylsilyl (TBS) ether and subsequent deprotection of the primary triethylsilyl (TES) ether under mildly acidic conditions gave primary alcohol 14b in good yield.

Compound 14b was also synthesized directly from compound 10 in a single flask, TMS-protected vinyl bromide 15 was used. Exposure of aldehyde 10 to standard CrCl₂/NiCl₂ conditions in the presence of 15 gave a smooth coupling to the corresponding chromium alkoxide (not shown) with no primary TMS desilylation observed. This chromium alkoxide was then cleanly silylated in situ as the TBS ether using TBS-triflate followed by mild acid quench to selectively cleave the primary TMS ether providing desired allylic alcohols 14a and 14b in a single flask from cyano aldehyde 10 in good yield. The separable epimeric mixture of products were taken on individually through the remainder of the sequence to determine their relative stereochemistry:

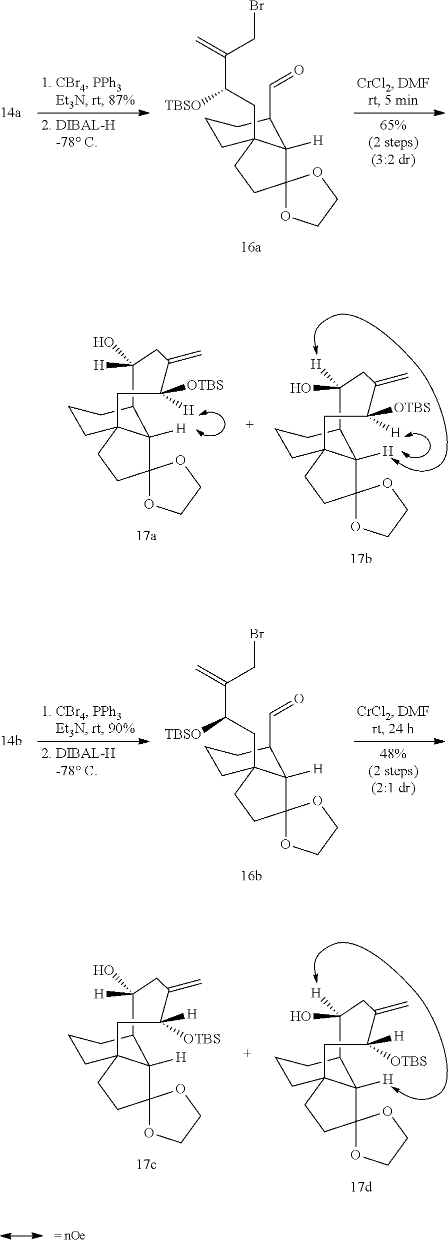

Conversion of 14a or 14b to the allylic bromide under CBr₄ conditions and subsequent nitrile reduction using diisobutylaluminum hydride (DIBAL-H) afforded aldehydes 16a and 16b over the two steps. With allyl bromo aldehydes 16a and 16b in hand, intramolecular NHK cyclization was undertaken. Surprisingly, exposure of a single C11 epimer of 16 to CrCl₂ at room temperature in dimethylformamide (DMF) rapidly induced cyclization within minutes to forge the desired propellane structure of pleuromutilin (17a/b and 17c/d).

In an experiment acylation of the secondary hydroxyl using a trityl-protected glycolate derivative proceeded without incident followed by acid-induced global deprotection to provide a targeted scaffold in good yield. This established a 10 step sequence to the functionalized propellane framework of pleuromutilin from known enone 5.

Two stage transformations were conducted as shown below to achieve pleuromutilin analogs:

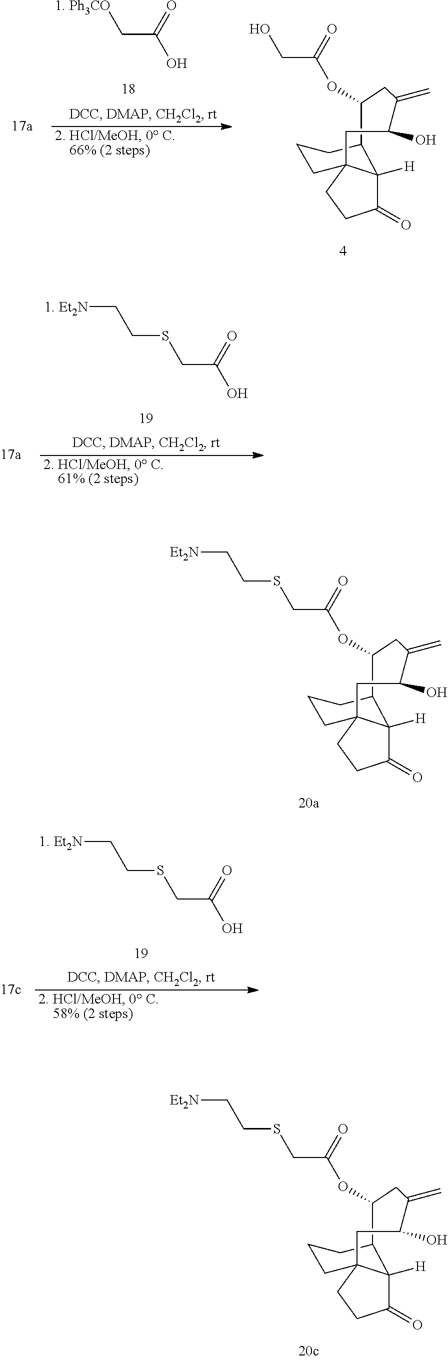

The couplings of compounds 17a and 17c with either the pleuromutilin-like glycolic acid derivative 18 or the tiamulin-like carboxylic acid 19 were mediated by dicyclohexylcarbodiimide (DCC) and followed by straightforward acid-induced deprotections to yield the novel screening candidates 4, 20a, and 20c for structure activity relationship studies (SAR studies).

Additional antitubercular screening candidates were produced from tricyclic alkenes 21a and 21b:

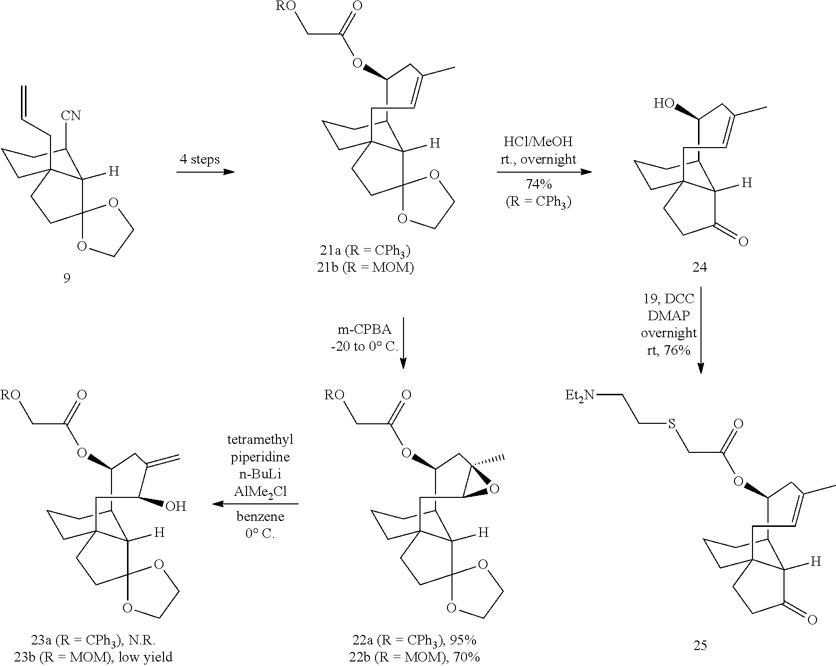

A straightforward, two-fold deprotection of compound 21a afforded tricyclic alcohol 24, which was subsequently joined with carboxylic acid 19 in the presence of DCC to give the novel, tiamulin-like ester 25. It was also possible to epoxidize the trisubstituted alkenes in compounds 21a and 21b with high margins of diastereoface selectivity; however, a subsequent effort to affect a base-induced epoxide ring opening of 22a to compound 23a was unsuccessful and an attempt to achieve an analogous conversion of 22b to 23b was only partially successful. The chromium-mediated, reductive cyclization strategy described herein is especially effective at producing compounds with the type of constitution embodied in compound 4 and its relatives.

To evaluate the antitubercular efficacies of tricyclic esters 4, 20a, 20c, and 25, their respective effects on the growth of M. tuberculosis mc²7000 were measured utilizing the Microplate Alamar Blue assay (Table 1). The results with pleuromutilin and tiamulin showed low micromolar MIC values, in accord with a previous report with Mtb strains. The compounds with the tiamulin-like ester side chain displayed higher inhibitory activity against Mtb mc²7000 (1 vs. 3; 4 vs. 20a). In relation to tiamulin, the tiamulin-like tricycle 20a displayed an attenuated activity. However, the promising efficacy of analog 20c, a compound with the tiamulin type C14 ester side chain and unnatural relative stereochemistry at C11; was intriguing. This scaffold also lacks the peripheral methyl groups as well as the C12 quaternary stereocenter and yet it compares favorably to tiamulin (3) in its inhibitory action against Mtb mc²7000. Another unanticipated finding was the moderate, yet significant, level of activity of compound 25, a substance that lacks the polar hydroxyl function at C11 and possesses unnatural relative stereochemistry at C14.

TABLE 1
MIC values for pleuromutilin family members against Mtb
mc²7000.

| Compound | mc²7000 7H9 MIC (ug/mL)^{a} |
|---|---|
| Pleuromutilin (1) | 25-50 |
| tiamulin (3) | 12-25 |
| 4 | >200 |
| 20a | 50-100 |
| 20c | 12-25 |
| 25 | 50 |

^{a}Each MIC value was reported as a range of experimentally assayed compound concentrations that bounds the mean for three independent determinations.

The NHK strategy permits the synthesis of chromatographically separable diastereomers, epimeric at C11 and C14, and allows for a probe of the effect of stereochemistry at these carbon centers on biological activity.

Whole-cell assays were conducted in liquid cultures of M tuberculosis mc²7000, which was diluted to OD₆₀₀=0.01 and grown for 5 days on 7H9 media supplemented with 30 mg/mL panthotenate, 0.2% dextrose, and 0.05% tyloxapol at 37° C. in the presence of a serial dilution gradient on inhibitors in 96-well plates. Cellular viability was probed by staining with Alamar Blue, which turns from blue (resazurin form) to pink (resorufin form) through reduction by actively respiring bacteria. The absorbance at peaks for the oxidized and reduced forms was read spectrophotometrically on a POLARstar Omega plate reader, and the difference between them represented the extent of growth. Raw data were scaled on a plate-by-plate basis using rifampicin (40 mM) and DMSO injected wells as positive and negative controls, respectively, for linear scaling. The minimal concentration of an inhibitor, resulting in the same growth inhibition as a positive control, was recorded as the MIC value. As used herein, “biologically active” means that a compound has a negative effect on bacterial growth as assessed by the above assay.

Interestingly, each simplified scaffold bearing the tiamulin side-chain exhibited similar activity to that of tiamulin, regardless of the stereochemical configurations at C₁₁ and C₁₄. These data also suggest that the C₅ and C₆ methyl groups may be omitted without affecting biological activity. Furthermore, the metabolically labile C₁₂ vinyl substituent of pleuromutilin, which is known to be susceptible to P450 oxidation, does not seem to be necessary for activity.

With two successful strategies for synthesizing simplified pleuromutilin scaffolds in hand and biological activity for several novel compounds, along with a concise, adaptable, and stereocontrolled total synthesis of pleuromutilin and pleuromutilin analogs; an increased size of the pleuromutilin class of antibiotics may be provided. In addition, substitution patterns on the pleuromutilin scaffold that correlate with high therapeutic efficacy may be provided.

### Example 3

### Synthesis of Pleuromutilin and Pleuromutilin Analogs May Begin with an Asymmetric Construction of Dibromide 63

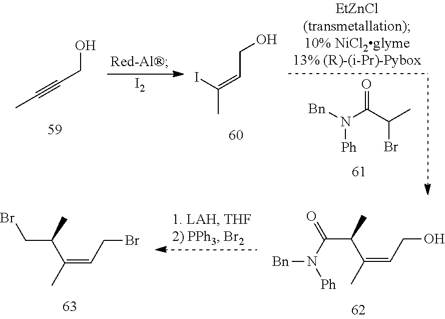

Standard Red-Al® reduction of 2-butyn-1-ol and iodine quench affords the known (Z)-vinyl iodide 60. Metal-halogen exchange with EtZnCl from 60 to 61 could be followed by a catalytic, asymmetric Negishi coupling with a-bromo amide 61 under Fu's conditions to provide allylic alcohol 62. Subsequent amide reduction and a two-fold bromination could furnish dibromide 63.

A sequential union of dibromide 63, isopropenyl cuprate 65, and cyclopentenone 64 may provide an advanced enol triflate 66:

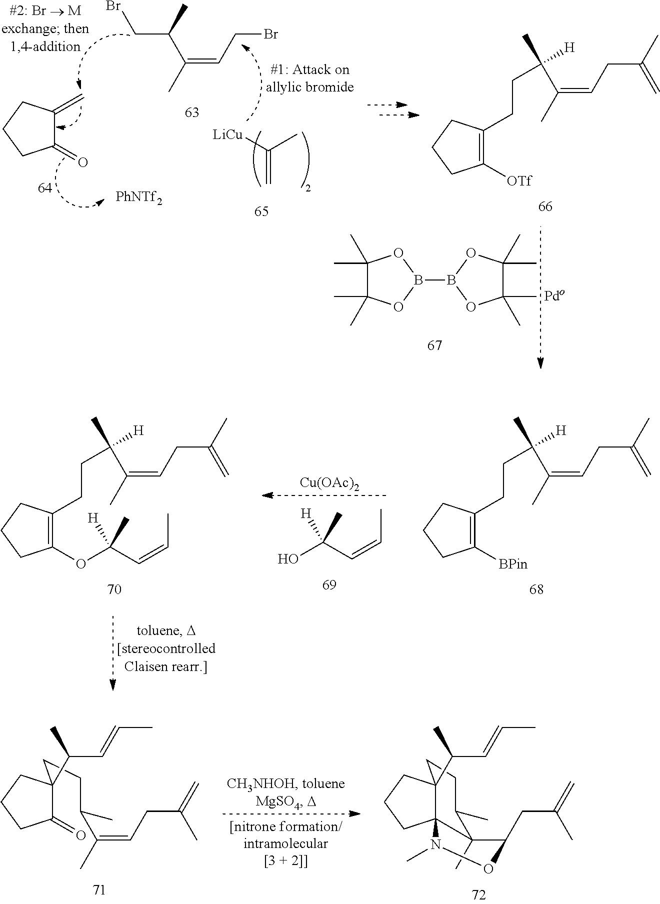

Selective displacement of the allylic bromide in 63 would afford an intermediate (not shown) that could be converted to an organocuprate reagent. 1,4-Addition of this pre-formed species to cyclopentenone 64 followed by trapping with N-phenyltriflimide is may furnish enol triflate 66.

Palladium mediated borylation could then be followed by a copper (II) promoted alkoxylation with homoallylic alcohol 69 to provide allyl vinyl ether 70. Thermally-induced and stereocontrolled Claisen rearrangement could then provide ketone 71 and establish a C₉-C₄₀ vicinal stereorelationship. This sequence could be capped by condensation with hydroxylamine and a concomitant intramolecular nitrone/alkene [3+2] cycloaddition to simultaneously establish the crowded C₄-C₅ bond and the oxygen-bearing C₁₄ stereocenter. The isoxazolidine ring arising from this reaction could serve the synthesis by reducing conformational freedom and provides an option for elaborating the C₃ ketone of pleuromutilin. This plan for constructing compound 72 has only 5 or 6 steps from building blocks 63, 64, and 65.

It may be possible to transform 72 to 73:

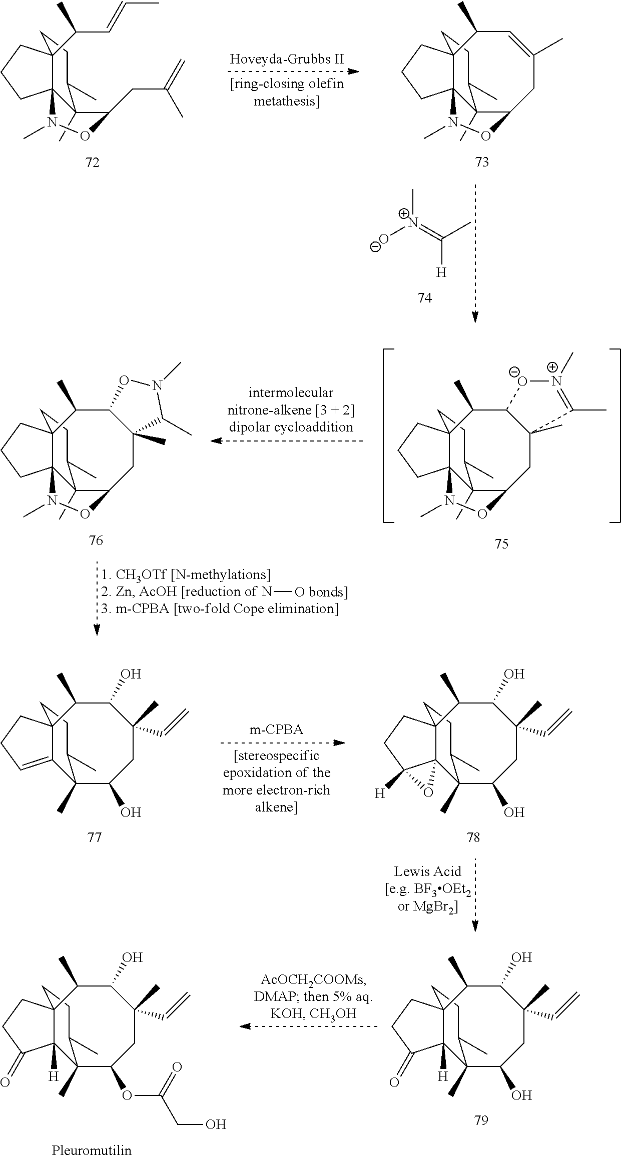

In some instances, when nitrone 74 engages the newly formed alkene in a regio- and stereo-face selective [3+2] dipolar cycloaddition (see 75), it may be possible to simultaneously manipulate the two isoxazolidine rings in compound 76 via a reaction sequence featuring a two-fold Cope elimination (76→77). This strategy is attractive because it would quickly install the needed groups at C₁₁ and C₁₂ and forge a relatively electron-rich C₃-C₄ alkene that may be converted to the C₃ ketone of pleuromutilin by the method of Boeckman (see 77→78→79). See Boeckman, R. K.; Springer, D. M.; Alessi, T. R. “Synthetic studies directed toward naturally occurring cyclooctanoids. 2. A stereocontrolled assembly of (±)-pleuromutilin via a remarkable sterically demanding oxy-Cope rearrangement” J. Am. Chem. Soc. 1989, 111, 8284-8286, which is incorporarted herein by reference as if fully set forth. A final, site-selective acylation of 79, again by the method of Boeckman, would complete synthesis of pleuromutulin. This 18-step plan for synthesis would be significantly shorter than previously published efforts; it could also be the basis for a serious effort to significantly expand the family of compounds sharing pleuromutilin's tricyclic scaffold.

A Paterno-Büchi photocycloaddition of chloroacetaldehyde to the less crowded stereoface of alkene 73 may be used:

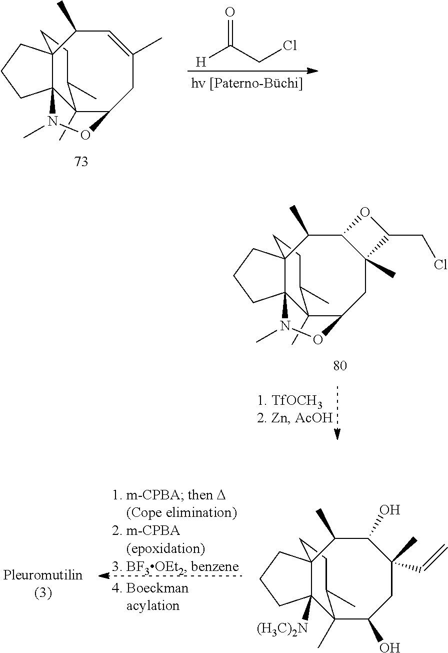

This cycloaddition may produce oxetane 80. After activation of the isoxazolidine by N-methylation, a two-fold reduction of the carbon-chlorine and weak nitrogen-oxygen bonds with zinc metal would reveal the interesting pleuromutilin system 81. From 81, pleuromutilin would be available by a pathway involving Cope elimination and a regio- and stereoselective oxidation of the electron-rich C₃-C₄ alkene.

A pleuromutilin endgame could be implemented as below:

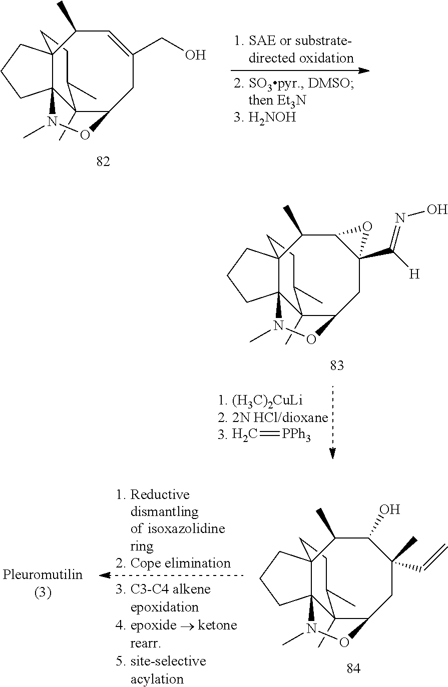

Allylic alcohol 82, which could arise from an allylic oxidation of compound 73, would be a substrate for a stereoface-selective epoxidation reaction, using the method of Sharpless if needed. See Katsuki, T.; Martin, V. S. “Asymmetric epoxidation of allylic alcohols: The Katsuki-Sharpless epoxidation reaction” Org. React. 1996, 48, 1-299, which is incorporated herein by reference as if fully set forth. A Parikh-Döering oxidation of the resultant alcohol and subsequent oxime formation could then provide epoxy oxime 83. The methyl group at the more crowded C₁₂ position may be introduced as shown above. A reaction of 83 with lithium dimethyl cuprate could lead toward pleuromutilin, although though a range of novel C₁₂ analogs may be provided by reacting 83 with different kinds of lithium diorganocuprate reagents. An elaboration of compound 84, which possesses the all-carbon quaternary stereocenter, may be provided by oxime hydrolysis and Wittig homologation. Sequences similar to those used above involving reduction, Cope elimination, epoxidation, rearrangement, and acylation could be utilized in the synthesis of pleuromutilin and analogs thereof.

### Example 4

Site-selective “point mutations” at various positions on the pleuromutilin scaffold may be provided. With respect to pleuromutilin's affinity for the 50S ribosomal subunit in bacteria, which is at the heart of its antibiotic activity, it is known that the encircled functions in 85 make bonding interactions with the ribosome and are important.

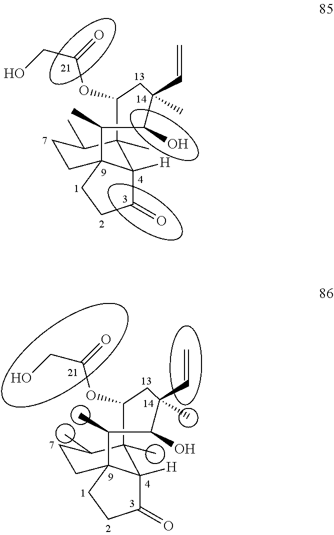

Although, these positions have been shown to be “critical” for biological activity in the past, the examples provided herein demonstrate that modification can be tolerated. For example, the hydroxyl at position C11 can be presented in either configuration. Based on these unexpected results, any position of pleuromutilin, or a pleuromutilin scaffold may be modified in a pleuromutilin analog. In addition, a pleruomutilin analog may be modified to provide a further analog at any position.

The promising in vitro activities of the simplified pleuromutilin analogs shown above indicate a general class of pleuromutilin analogs that may be provided. Through continued advances in synthesis, all six of the encircled groups in 86 could be provided and systematically varied to produce new compounds that are unavailable by semi-synthesis. Pleuromutilin and its relatives offer a scaffold that can be used to contribute new members to this class of antibiotics.

### Example 5

Summaries of selected synthesis schemes herein are provided below:

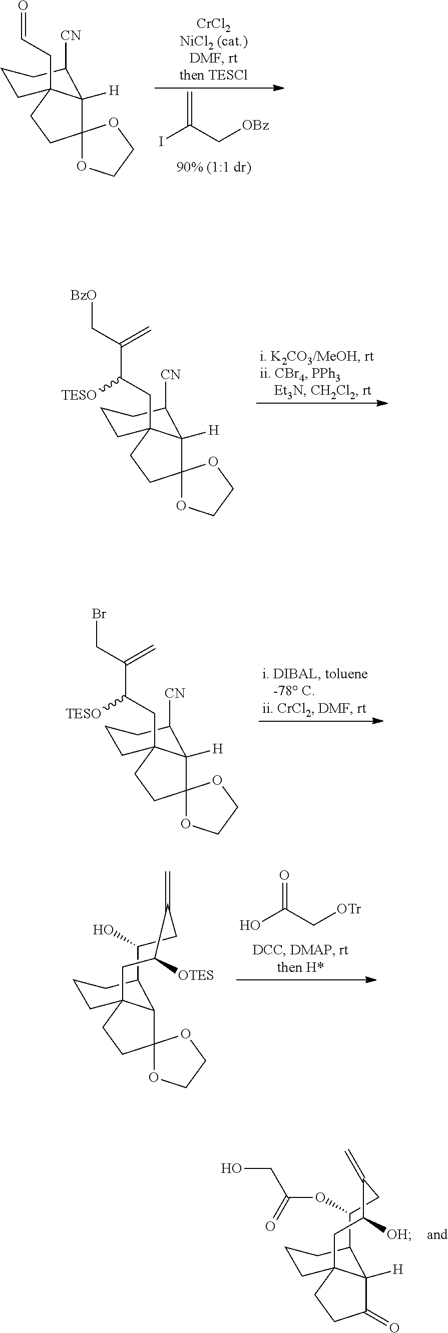

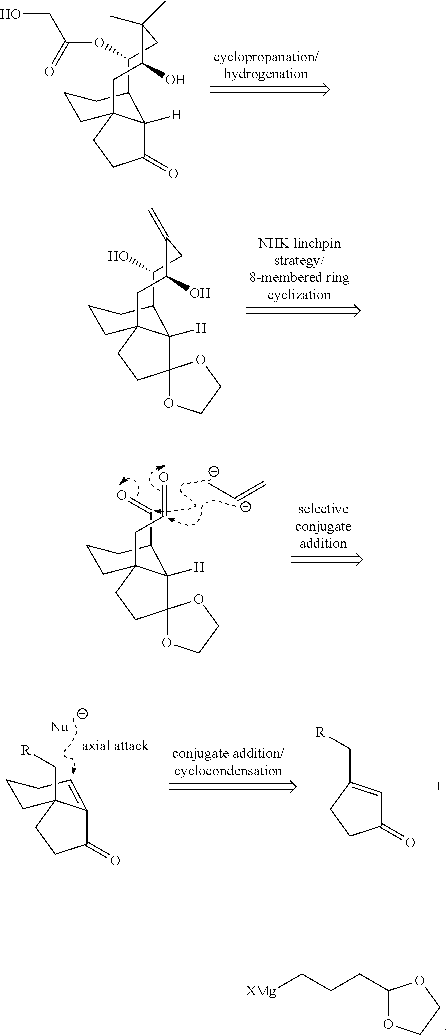

A target compound of synthesis may be

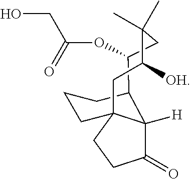

### Example 6

Examples of using an antibiotic to probe ribosomal structure and function relationships can be found in Marconi et al. “Identification of a rRNA/Chloramphenicol Interaction Site within the Peptidyltransferase Center of the 50S Subunit of the Escherichia coli Ribosome” (1990) J. Biol. Chem. 265(14): 7894-7899, which is incorporated herein by reference as if fully set forth. Any one or more pleuromutilin scaffold or analog herein may replace the chloramphenicol in experiments like those in Marconi et al. to probe ribosomal structure and function relationships, or pleuromutilin interactions with the ribosome.

### Example 7

General Methods. All reactions were carried out under an atmosphere of argon with magnetic stirring unless otherwise indicated. Palladium (II) acetate was purchased from Gelest. In other cases, commercial reagents of high purity were purchased from either Aldrich or Acros and used without further purification. Tetrahydrofuran (THF), dichloromethane (CH₂Cl₂), toluene, benzene, ether (Et₂O), acetonitrile (CH₃CN), triethylamine (NEt₃), and pyridine were dried by passing through activated alumina columns. Reactions were monitored by thin layer chromatography (TLC) carried out on 0.25 mm Whatman silica gel plates Partisil K6F (60 Å) using UV light as a visualizing agent and aqueous potassium permanganate or ethanolic p-anisaldehyde solution and heat as developing agents. Silica gel from SiliCycle silicaFlash P60 40-63 μm (230-400 mecsh) or from Dynamic Adsorbent Inc 32-63 μm was used for flash column chromatography.

Instrumentation. FT-IR spectra were obtained on a Perkin-Elmer Paragon 500. Nuclear magnetic resonance (NMR) spectra were obtained on a 500 MHz Bruker AVANCE spectrometer and calibrated to the residual solvent peak. Coupling constant values were extracted assuming first-order coupling and are given in Hz. The multiplicities are abbreviated as follows: s=singlet, d=doublet, t=triplet, q=quartet, m=multiplet, and br=broad signal. High resolution mass spectra were obtained on a Kratos MS 50 using electrospray ionization (ESI).

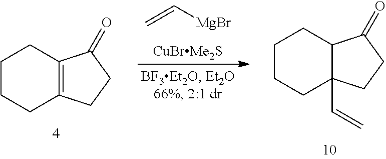

3a-vinyloctahydro-1H-inden-1-one (10): To a suspension of copper(I) bromide dimethyl sulfide complex (CuBr.Me₂S, 10.4 g, 50 mmol, 2 eq.) in 150 mL ether, vinylmagnesium bromide (1.0 M in THF, 100 mL, 100 mmol, 4 eq.) was added dropwise at −40° C. and the mixture turned dark. After stirring at −40° C. for 1.5 hours, the mixture was cooled to −78° C. and BF₃.Et₂O (7.7 mL, 7.1 g, 50 mmol, 2 eq.) was added dropwise. 10 minutes later, enone 41 (3.4 g, 25 mmol, 1 eq.) in 50 mL ether was added dropwise at −78° C. The mixture was stirred at −78° C. for 5 hours, and then was slowly warmed to room temperature. 200 mL of saturated aqueous NH₄Cl was added, and the mixture was extracted with ether (4×100 mL). The combined organic layers were washed with saturated aqueous NH₄Cl twice and brine once, then dried over MgSO₄ and filtered through Celite before removal of the solvent under reduced pressure. The residue was purified by chromatography on silica gel (ether/pentane: 1/19) to give ketone 10 (2.7 g, 66%) as an oil and as a 2:1 mixture of diastereomers. Major diastereomer: ¹H-NMR (500 MHz, CDCl₃): δ 5.92 (1H, dd, J=17.5, 10.8), 5.13 (1H, d, J=10.8), 5.08 (1H, d, J=17.5), 2.37-2.23 (2H, m), 2.17 (1H, s), 2.01 (1H, dd, J=13.5, 2.8), 1.84-1.69 (2H, m), 1.60 (1H, t, J=14.2), 1.52-1.42 (3H, m), 1.40-1.29 (1H, m), 1.28-1.20 (1H, m), 1.11-1.00 (1H, m). ¹³C-NMR (500 MHz, CDCl₃): δ 219.7, 145.0, 112.9, 53.8, 44.8, 34.9, 32.9, 32.1, 22.7, 21.7, 21.3. IR (neat): ν 2932, 2859, 1741, 1638, 1448 cm⁻¹. HRMS (ESI+): calculated for C₁₁H₁₇O ([M+H]⁺) 165.12794, found 165.12746.

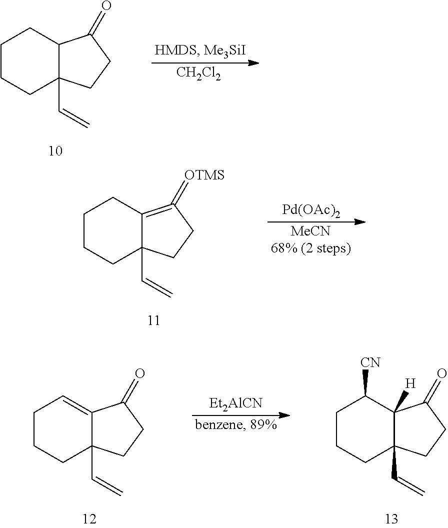

3a-vinyl-2,3,3a,4,5,6-hexahydro-1H-inden-1-one (13): Hexamethyldisilazane (HMDS, 26 mL, 20 g, 125 mmol, 7.6 eq.) was added dropwise to a solution of ketone 10 (2.7 g, 16.5 mmol, 1 eq.) in 50 mL dichloromethane at −20° C., followed by dropwise addition of trimethylsilyl iodide (TMSI, 8.8 mL, 13 g, 63 mmol, 3.8 eq.). The mixture was slowly warmed to room temperature and stirred for 4 hours. The dark greenish mixture was cooled back to −20° C., and another portion of HMDS (10 mL, 8.0 g, 50 mmol, 3 eq.) and TMSI (3.5 mL, 5.1 g, 25 mmol, 1.5 eq.) was added dropwise. The system was allowed to warm to room temperature and stirred overnight. 300 mL of saturated aqueous NaHCO₃ was added to the orange mixture at 0° C. before extraction with ether. The combined organic layers were washed with brine and then dried over MgSO₄ before removal of the solvent under reduced pressure to give crude silyl enol ether 11. Palladium (II) acetate (Pd(OAc)₂, 3.7 g, 16.5 mmol, 1 eq.) was added to a solution of the crude 11 in 50 mL acetonitrile in one portion at room temperature. After the solution was stirred for 2.5 hours, Pd(OAc)₂ (1.9 g, 8.5 mmol, 0.5 eq.) was added in three portions during the next 2 hours. Ether, saturated aqueous Na₂S₂O₃ and an excess of Na₂S₂O₃ solid were added after the mixture was filtered through Celite, and the resultant two-phase solution was stirred overnight until the organic layer turned colorless. After filtration through Celite, the mixture was extracted with ether, and the combined organic layers were washed with brine twice, and dried over MgSO₄ before removal of the solvent under reduced pressure. The residue was purified by chromatography on silica gel (ether/pentane:1/10) to give enone 12 (1.8 g, 68%) as a volatile oil. Due to this compound's volatility, not all the ether could be removed, and the yield was calculated by integration of the ¹H-NMR spectrum; the mixture was used directly in the next step. ¹H-NMR (500 MHz, CDCl₃): δ 6.80 (1H, d, J=3.2), 5.74 (1H, dd, J=17.2, 10.3), 5.15 (1H, d, J=10.3), 4.75 (1H, dd, J=17.3, 1.0), 2.36-2.05 (5H, m), 1.91 (1H, dd, J=12.6, 3.1), 1.68-1.53 (3H, m), 1.34-1.23 (1H, m). ¹³C-NMR (500 MHz, CDCl₃): δ 207.0, 143.8, 141.4, 134.2, 117.0, 46.6, 35.2, 34.4, 33.9, 25.5, 17.9.

(3aS,4R,7aS)-3-oxo-7a-vinyloctahydro-1H-indene-4-carbonitrile (13): To a solution of enone 12 (160 mg, 1.0 mmol, 1 eq.) in 5 mL of benzene was added diethylaluminum cyanide (1.0 M in toluene, 3.0 mL, 3.0 mmol, 3 eq.) dropwise at −10° C. The solution was slowly warmed to room temperature over 3 hours, and the reaction mixture turned red. Saturated aqueous sodium potassium tartrate (Rochelle's salt) was added, and the reaction was stirred at room temperature for 30 minutes. The mixture was extracted with ether, and the combined organic layers were dried over MgSO₄ before removal of the solvent under reduced pressure. The residue was purified by chromatography on silica gel (ether/pentane:1/3) to give ketone 13 (166 mg, 89%, >95:5 dr) as a solid. ¹H-NMR (500 MHz, CDCl₃): δ 6.14 (1H, dd, J=17.4, 10.9), 5.27 (1H, d, J=10.8), 5.26 (1H, d, J=17.6), 3.42 (1H, dd, J=3.6, 0.9), 2.46-2.25 (3H, m), 1.94-1.71 (5H, m), 1.60-1.52 (1H, m), 1.28 (1H, dddd, J=8.1, 4.8, 4.0, 1.6), 1.21-1.13 (1H, m). ¹³C-NMR (500 MHz, CDCl₃): δ 214.5, 142.8, 121.8, 114.5, 55.4, 44.1, 33.9, 33.0, 30.3, 25.4, 23.9, 18.2. M.P.: 41-42° C. IR (neat): ν 3084, 2940, 2868, 2237, 1745, 1452 cm⁻¹. HRMS (ESI+): calculated for C12H₁₆NO ([M+H]⁺) 190.12319, found 190.12271.

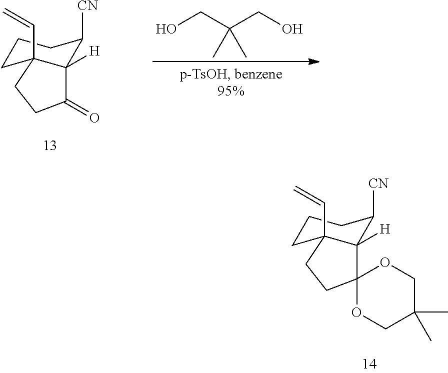

(3a′S,7′R,7a′S)-5,5-dimethyl-3a′-vinyloctahydrospiro[[1,3]dioxane-2,1′-indene]-7′-carbonitrile (14): A solution of 13 (0.51 g, 2.7 mmol, 1 eq.), 2,2-dimethylpropane-1,3-diol (1.4 g, 13.5 mmol, 5 eq.), and p-toluenesulfonic acid (0.26 g, 1.35 mmol, 0.5 eq.) in 25 mL benzene was stirred at reflux using a Dean-Stark trap for removal of water. Upon consumption of 13 by TLC, saturated aqueous NaHCO₃ (50 ml) was added at 0° C., and the aqueous layer was separated and extracted with ether. The combined organic layers were dried over MgSO₄ before removal of the solvent under reduced pressure. The residue was purified by chromatography on silica gel (ether/hexane:1/9) to give ketal 14 (0.71 g, 95%). ¹H-NMR (500 MHz, CDCl₃): δ 5.99 (1H, dd, J=17.5, 10.8), 5.13 (1H, d, J=17.6), 5.11 (1H, d, J=10.8), 3.54 (2H, t, J=11.6), 3.45-3.34 (2H, m), 3.23-3.14 (1H, m), 2.19 (1H, s), 2.05 (1H, ddd, J=13.2, 9.0, 4.0), 2.00-1.86 (2H, m), 1.75-1.61 (4H, m), 1.55-1.42 (3H, m), 1.19 (3H, s), 0.72 (3H, s). ¹³C-NMR (500 MHz, CDCl₃): δ 145.4, 123.8, 112.8, 109.0, 72.5, 71.4, 54.7, 44.2, 36.7, 30.1, 30.0, 28.6, 25.9, 23.9, 22.8, 22.2, 18.4. IR (neat): ν 3083, 2951, 2868, 2237, 1638, 1471, 1396 cm⁻¹. HRMS (ESI+): calculated for C₁₇H₂₆NO₂ ([M+H]⁺) 276.19635, found 276.19539.

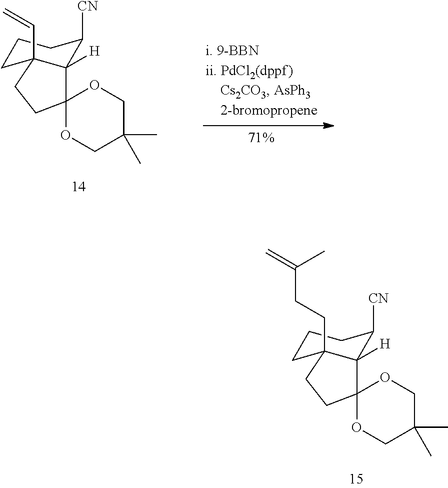

(3a′R,7′R,7a′S)-5,5-dimethyl-3a′-(3-methylbut-3-enyl) octahydrospiro[[1,3]dioxane-2,1′-indene]-7′-carbonitrile (15): 9-borabicyclo(3.3.1)nonane (9-BBN, 0.5 M in THF, 2.67 mL, 1.33 mmol, 2 eq.) was added to ketal 14 (183 mg, 0.67 mmol, 1 eq.) at room temperature under argon. The solution was stirred for 3.5 hours until 14 was consumed by TLC. In a separate flask, a solution of 2-bromopropene (0.29 mL, 0.40 g, 3.3 mmol, 5 eq.) in 10 mL DMF was stirred, and cesium carbonate (Cs₂CO₃, 0.54 g, 1.67 mmol, 2.5 eq.), triphenylarsine (AsPh₃, 41 mg, 0.13 mmol, 0.2 eq.), [1,1-bis(diphenylphosphino) ferrocene]dichloropalladium(II) (PdCl₂(dppf), 97 mg, 0.13 mmol, 0.2 eq.), and H₂O (0.5 mL) were added successively at room temperature. The mixture was stirred for 5 minutes, and the crude alkyl borane from 14 was added dropwise. The mixture was stirred overnight and then quenched with saturated aqueous NH₄Cl. The mixture was extracted with ethyl acetate. The combined organic layers were washed with water and dried over MgSO₄ before removal of the solvent under reduced pressure. The residue was purified by chromatography on silica gel (ethyl acetate/hexane:1/20) to give 13 (150 mg, 71%). ¹H-NMR (500 MHz, CDCl₃): δ 4.70 (1H, s), 4.68 (1H, s), 3.53 (2H, t, J=11.1), 3.43-3.33 (2H, m), 3.21-3.13 (1H, m), 2.04-1.85 (7H, m), 1.76-1.44 (10H, m), 1.35 (1H, td, J=13.8, 4.2), 1.17 (3H, s), 0.71 (3H, s). ¹³C-NMR (500 MHz, CDCl₃): δ 146.2, 124.1, 109.9, 109.2, 72.5, 71.3, 54.7, 41.2, 36.7, 34.7, 32.8, 30.1, 29.7, 28.2, 26.1, 23.6, 22.8, 22.8, 22.2, 18.0. IR (neat): ν 2941, 2867, 2235, 1648, 1457, 1133, 1100 cm⁻¹. HRMS (ESI+): calculated for C₂₀H₃₂NO₂ ([M+H]⁺) 318.24330, found 318.24323.

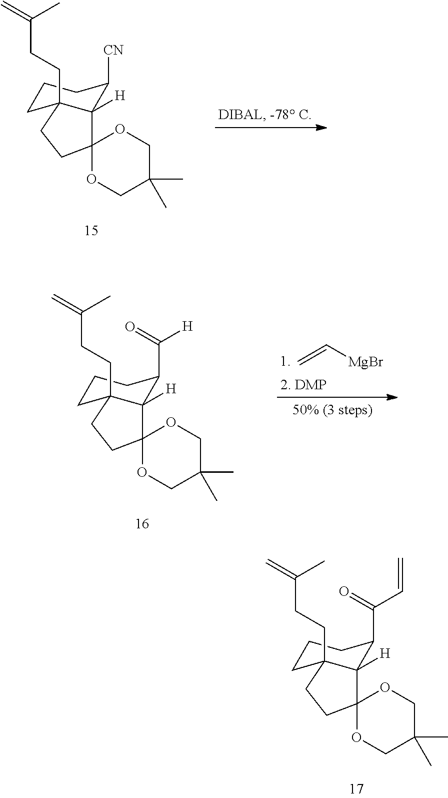

1-((3a′R,7′R,_{7a′S})-5,5-dimethyl-3a′-(3-methylbut-3-enyl)octahydrospiro[[1,3]dioxane-2,1′-indene]-7′-yl)prop-2-en-1-one (17): To a solution of nitrile 15 (13 mg, 0.041 mmol, 1 eq.) in 1.5 mL toluene was added diisobutylaluminum hydride (DIBAL, 1.0 M in toluene, 0.05 mL, 0.05 mmol, 1.2 eq.) dropwise at −78° C. Upon completion by TLC, 0.3 mL methanol was added at −78° C. The bath was removed, and 0.5 mL aqueous citric acid (1.0 M) was then added. The reaction was stirred for 15 minutes at room temperature. The organic layer was washed with saturated NaHCO₃ and brine and dried over MgSO₄ before removal of the solvent under reduced pressure, giving crude aldehyde 16 in quantative yield without purification. To a solution of crude 16 in 2 mL THF was added vinylmagnesium bromide (1.0 M in THF, 0.045 mL, 0.045 mmol, 1.1 eq.) at −78° C. Upon consumption of 16, saturated aqueous NH₄Cl was added at −78° C. After extraction with ether, drying, and removal of the solvent, the crude allylic alcohol mixture was subjected to a solution of Dess-Martin periodinane (DMP, 52 mg, 0.12 mmol, 3 eq.) and pyridine (0.1 mL) in 2 mL dichloromethane. After quenching with NaHCO₃ and extraction with dichloromethane, the mixture was dried over Na₂SO₄ before removal of the solvent under reduced pressure. The residue was purified by chromatography on silica gel (ethyl acetate/hexane:1/19) to give 17 (7 mg, 50% over 3 steps). ¹H-NMR (500 MHz, CDCl₃): 6.42 (1H, dd, J=17.4, 10.6), 6.22 (1H, d, J=17.4), 5.65 (1H, d, J=10.5), 4.64 (2H, s), 3.48 (1H, d, J=11.2), 3.44 (1H, d, J=11.1), 3.33 (1H, d, J=11.2), 3.25 (1H, d, J=11.0), 2.97 (1H, m), 2.33 (1H, d, J=7.9), 2.03-1.80 (4H, m), 1.75-1.31 (13H, m), 1.03 (3H, s), 0.67 (3H, s). ¹³C-NMR (500 MHz, CDCl₃): δ 203.5, 146.9, 136.1, 126.7, 109.8, 109.4, 71.7, 71.7, 55.1, 43.8, 41.7, 38.0, 33.1, 32.6, 30.8, 30.3, 28.9, 25.8, 22.9, 22.8, 22.3, 19.7. IR (neat): ν 2935, 2865, 1698, 1456, 1397, 1133, 1110 cm⁻¹. HRMS (ESI+): calculated for C₂₂H₃₅O₃ ([M+H]⁺) 347.25862, found 347.25838.

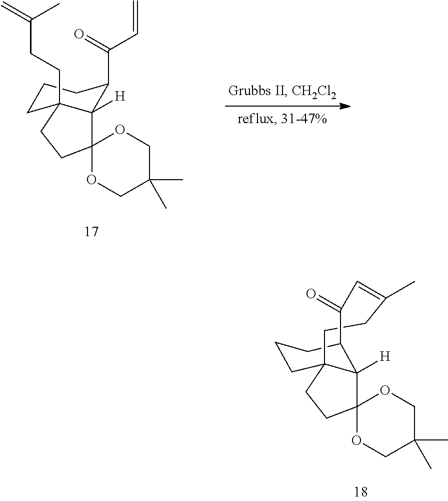

tricyclic pleuromutilin derivative 18: To a solution of enone 17 (7 mg, 0.02 mmol) in 10 mL dichloromethane was added Grubbs' second generation olefin metathesis catalyst (Grubbs II, 2 mg, 0.002 mmol, 0.1 eq.) at room temperature. The solution was refluxed for 24 hours, and additional Grubbs II was added during this period (2×2 mg). Saturated aqueous NaHCO₃ was added, and the solution was extracted with dichloromethane. The organic layers were dried over MgSO₄ before removal of the solvent under reduced pressure. The residue was purified by chromatography on silica gel (ethyl acetate/hexane:1/9) to give 18 (2-3 mg, 31-47%). ¹H-NMR (500 MHz, CDCl₃): δ 5.66 (1H, s), 3.57 (1H, d, J=10.9), 3.55 (1H, d, J=10.9), 3.42 (1H, dd, J=11.1, 2.8), 3.38 (1H, dd, J 11.1, 2.8), 2.65 (1H, s), 2.61-2.56 (1H, m), 2.26-2.05 (5H, m), 1.94 (1H, tt, J=13.3, 5.1), 1.85-1.72 (5H, m), 1.65 (1H, td, J=13.3, 4.6), 1.45 (1H, qd, J=13.7, 9.5), 1.37-1.30 (1H, m), 1.26-1.21 (2H, m), 1.15 (3H, s), 1.04 (1H, d, J=13.7), 0.71 (3H, s). ¹³C-NMR (500 MHz, CDCl₃): δ 210.4, 148.5, 124.4, 110.1, 72.8, 71.0, 49.9, 45.9, 41.0, 33.2, 32.0, 31.9, 31.0, 30.1, 29.2, 27.1, 22.6, 22.2, 20.9, 17.7. IR (neat): ν 2944, 2867, 2360, 1666, 1457 cm⁻¹. HRMS (ESI+): calculated for C₂₀H₃₁O₃ ([M+H]⁺) 319.22732, found 319.22691.

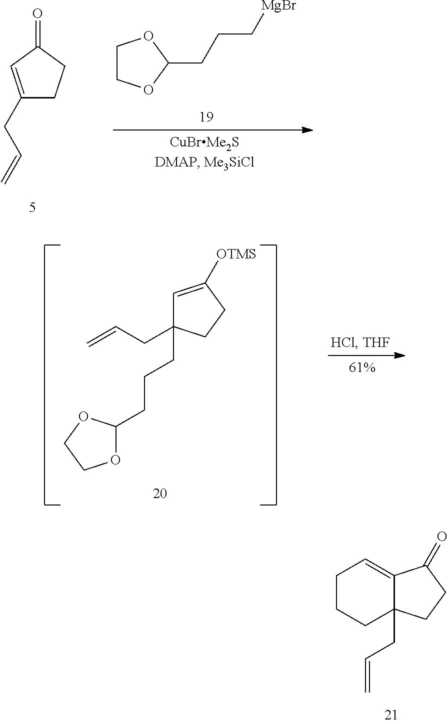

3a-allyl-2,3,3a,4,5,6-hexahydro-1H-inden-1-one (21): To a suspension of CuBr.Me₂S (5.06 g, 24.6 mmol, 1.5 eq.) in THF (100 ml) was added 4-dimethylaminopyridine (DMAP, 4.00 g, 32.8 mmol, 2.0 eq.) at −78° C. The green suspension was stirred for 5 minutes at −78° C. before dropwise addition of Grignard reagent 192 (2.5 M solution in THF, 20 ml, 50 mmol, 3.0 eq.). After 30 minutes of stirring, a solution of 53 (2.00 g, 16.4 mmol, 1 eq.) in THF (20 ml) and trimethylsilyl chloride (4.10 ml, 32.78 mmol, 2.0 eq.) were added successively at −78° C. The reaction was stirred for 1 hour at −78° C. and was allowed to warm to room temperature overnight. Upon consumption of 5 by TLC, aqueous HCl (5 M, 100 ml) was added, and the mixture was refluxed for 6 hours until the reaction was complete. Upon cooling to room temperature, the reaction was quenched by the addition of saturated aqueous NaHCO₃ (200 ml), followed by addition of ether (200 ml). The aqueous layer was separated and extracted with ether (2×100 ml). The combined organic layers were dried over MgSO₄ before removal of the solvent under reduced pressure. The residue was purified by chromatography on silica gel (ether/pentane:1/9) to give 21 (1.77 g, 61% from 5) as a pale orange oil. ¹H-NMR (500 MHz, CDCl₃): δ 6.64 (1H, t, J=3.7), 5.78 (1H, ddt, J=17.4, 10.2, 7.4), 5.11-5.05 (2H, m), 2.43-2.26 (2H, m), 2.24-2.07 (5H, m), 2.04 (1H, dt, J=13.0, 3.4), 1.74-1.68 (2H, m), 1.38 (1H, ddd, J=12.6, 9.6, 0.7), 1.11 (1H, td, J=12.6, 5.4). ¹³C-NMR (500 MHz, CDCl₃): δ 207.4, 145.4, 134.4, 132.7, 118.1, 41.6, 40.2, 35.2, 32.5, 32.0, 25.1, 17.7. IR (neat): ν 2935, 2870, 1718, 1651, 1458, 1419 cm⁻¹. HRMS (ESI+): calculated for C₁₂H₁₇O ([M+H]⁺) 177.12794, found 177.1266.

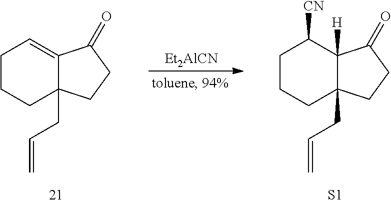

(3aS,4R,7aS)-7a-allyl-3-oxooctahydro-1H-indene-4-carbonitrile (S1): To a solution of 21 (1.77 g, 10 mmol, 1.0 eq.) in 50 mL toluene was added Et₂AlCN (1.0 M solution in toluene, 20 mL, 20 mmol, 2.0 eq.) dropwise at −10° C. The reaction was stirred at 0° C. and was monitored by TLC. Upon complete consumption of 21 by TLC (−3 hours), the solution was cooled to −20° C., and saturated aqueous Rochelle's salt (125 ml) was added. The bath was removed, and the reaction was stirred for 1 hour at room temperature. The aqueous layer was separated and extracted with ether (2×25 ml). The combined organic layers were dried over MgSO₄ before removal of the solvent under reduced pressure. The residue was purified by chromatography on silica gel (ethyl acetate/hexane:1/10) to give S1 (1.93 g, 94%, >95:5 dr). ¹H-NMR (500 MHz, CDCl₃): δ 5.91-5.77 (1H, m), 5.27-5.16 (2H, m), 3.39 (1H, d, J=2.3), 2.61 (1H, dd, J=14.0, 7.9), 2.49 (1H, dd, J=14.0, 6.9), 2.37-2.17 (3H, m), 1.91-1.48 (6H, m), 1.30-1.16 (1H, m), 1.07 (1H, td, J=13.8, 3.1). ¹³C-NMR (500 MHz, CDCl₃): δ 215.4, 133.2, 122.2, 119.8, 54.0, 41.4, 41.2, 33.6, 30.6, 25.5, 23.6, 17.6. IR (neat): ν 3076, 2938, 2067, 2236, 1744, 1639, 1453, 1409 cm⁻¹. HRMS (ESI+): calculated for C₁₃H₁₈NO ([M+H]⁺) 204.13884, found 204.13864.

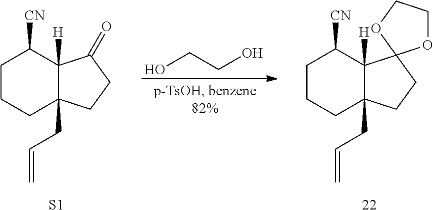

(3a′S, 7′R, 7a′S)-3a′-allyloctahydrospiro[[1, 3]dioxolane-2,1′-indene]-7′-carbonitrile (22): A solution of S1 (1.93 g, 9.5 mmol, 1.0 eq.), ethylene glycol (5.30 ml, 95 mmol, 10 eq.), and p-toluenesulfonic acid (900 mg, 4.75 mmol, 0.5 eq.) in benzene (150 ml) was refluxed using a Dean-Stark trap for removal of water. Upon consumption of S1 by TLC, saturated aqueous NaHCO₃ (50 ml) was added at 0° C., and the aqueous layer was separated and extracted with ether (50 ml). The combined organic layers were dried over MgSO₄ before removal of the solvent under reduced pressure. The residue was purified by chromatography on silica gel (ethyl acetate/hexane: 7.5/92.5) to give 22 (1.93 g, 82%). ¹H-NMR (500 MHz, CDCl₃): δ 5.84-5.71 (1H, m), 5.14-5.07 (2H, m), 4.00-3.83 (4H, m), 2.65 (1H, td, J=7.2, 4.7), 2.42 (1H, dd, J=13.9, 7.6), 2.24 (1H, dd, J=13.8, 7.3), 2.02-1.95 (2H, m), 1.92 (2H, dd, J=8.6, 7.1), 1.71-1.56 (2H, m), 1.56-1.36 (5H, m). ¹³C-NMR (500 MHz, CDCl₃): δ 134.4, 123.8, 118.6, 118.2, 65.1, 64.3, 51.5, 43.5, 42.7, 34.6, 32.0, 30.8, 27.2, 25.5, 18.7. IR (neat): ν 3075, 2236, 1638, 1455 cm⁻¹. HRMS (ESI+): calculated for C₁₅H₂₂NO₂ ([M+H]⁺) 248.16505, found 248.1652.

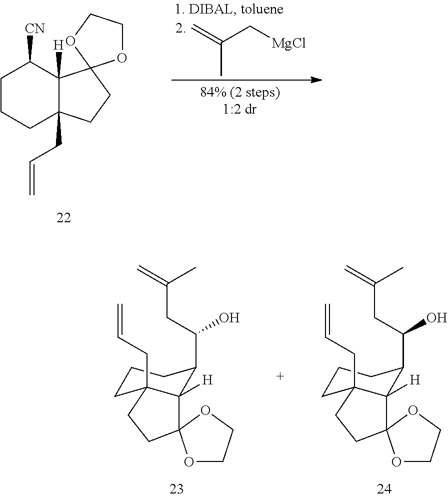

(S)-1-((3a′S,7′R,7a′S)-3a′-allyloctahydrospiro[[1,3]dioxolane-2,1′-indene]-7′-yl)-3-methylbut-3-en-1-ol (23) & (R)-1-((3a′S,7′R,7a′S)-3a′-allyloctahydrospiro[[1,3]dioxolane-2,1′-indene]-7′-yl)-3-methylbut-3-en-1-ol (24): To a solution of 22 (200 mg, 0.81 mmol, 1 eq.) in 15 mL toluene was added DIBAL (1.0 Min toluene, 2.45 mL, 2.45 mmol, 3 eq.) at −78° C. The solution was stirred for three hours at −78° C. before addition of 5 mL methanol and 10 mL 10% aqueous citric acid at −78° C. The mixture was slowly warmed to room temperature before extraction with ether. The combined organic layers were dried over MgSO₄ before removal of the solvent under reduced pressure to give the crude aldehyde. To a solution of the resultant aldehyde in 15 mL THF was added (2-methylallyl)magnesium chloride (0.5 M in THF, 1.7 mL, 0.85 mmol, 1.05 eq.) dropwise at −78° C. The solution was stirred for 10 minutes at −78° C., and saturated aqueous NH₄Cl was added at this temperature. The mixture was slowly warmed to room temperature and extracted with ether. The combined organic layers were washed with saturated aqueous NaHCO₃ and brine successively and dried over MgSO₄ before removal of the solvent under reduced pressure. The residue was purified by chromatography on silica gel (ethyl acetate/hexane:3/17) to give a mixture of 23 and 24 (206 mg, 84%, 1:2 dr). These two diastereomers were further separated by repeated chromatography (ethyl acetate/hexane:1/19).

(23): ¹H-NMR (500 MHz, CDCl₃): δ 5.86 (1H, td, J=17.0, 7.4), 5.09-5.00 (2H, m), 4.88 (1H, s), 4.81 (1H, s), 3.96-3.89 (3H, m), 3.78 (2H, dt, J=17.4, 10.5), 2.36 (1H, d, J=13.5), 2.26 (1H, dd, J=13.7, 7.6), 2.17 (1H, dd, J=13.6, 7.2), 2.10 (2H, s), 1.93 (1H, dd, J=13.1, 10.8), 1.82-1.60 (8H, m), 1.51-1.37 (4H, m), 1.37-1.23 (2H, m). ¹³CNMR (500 MHz, CDCl₃): δ 143.8, 136.0, 119.4, 117.4, 113.6, 69.5, 64.7, 63.7, 49.1, 44.9, 43.4, 41.6, 37.9, 34.5, 34.3, 31.6, 22.7, 22.4, 18.0. IR (neat): ν 3555, 3073, 2934, 1640, 1455 cm⁻¹. HRMS (ESI+): calculated for C₁₉H₃₀NaO₃ ([M+Na]⁺) 329.20926, found 329.20896.

(24): ¹H-NMR (500 MHz, CDCl₃): δ 5.87-5.75 (1H, m), 5.09-5.01 (2H, m), 4.85 (1H, s), 4.79 (1H, s), 3.95-3.80 (5H, m), 2.27-2.19 (2H, m), 2.13 (2H, ddd, J=13.9, 8.4, 5.8), 1.89 (2H, dd, J=12.5, 5.9), 1.81-1.72 (5H, m), 1.70-1.29 (9H, m). ¹³C-NMR (500 MHz, CDCl₃): δ 143.7, 135.8, 119.4, 117.5, 113.1, 69.3, 64.5, 63.8, 50.1, 45.2, 43.9, 41.8, 37.5, 34.1, 34.1, 31.6, 22.5, 20.7, 18.0. IR (neat): ν 3478, 3073, 2939, 1639, 1455 cm⁻¹. HRMS (ESI+): calculated for C₁₉H₃₀NaO₃ ([M+Na]±) 329.20926, found 329.20887.

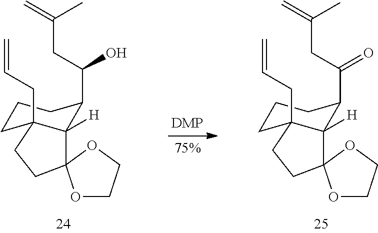

1-((3a′S,7′R,7a′S)-3a′-allyloctahydrospiro[[1,3]dioxolane-2,1′-indene]-7′-yl)-3-methylbut-3-en-1-one (25): To a solution of 24 (0.43 g, 1.4 mmol, 1.0 eq.) in 20 mL dichloromethane was added NaHCO₃ (1.2 g, 14 mmol, 10 eq.) and Dess-Martin periodinane (DMP, 0.78 g, 1.8 mmol, 1.3 eq.) at room temperature. The mixture was stirred for 1.5 hours and then saturated aqueous NaHCO₃ was added. After the organic layer turned transparent, the mixture was extracted with dichloromethane. The combined organic layers were dried over Na₂SO₄ before removal of the solvent under reduced pressure. The residue was purified by chromatography on silica gel (ethyl acetate/hexane:1/9) to give 25 (0.32 g, 75%). ¹H-NMR (500 MHz, CDCl₃): δ 5.89-5.71 (1H, m), 5.08-5.00 (2H, m), 4.95-4.92 (1H, m), 4.78 (1H, d, J=0.8), 3.92 (1H, ddd, J=8.2, 6.4, 3.4), 3.73 (1H, td, J=8.3, 6.7), 3.57 (1H, dt, J=8.5, 6.6), 3.40 (1H, td, J=6.7, 3.4), 3.31 (1H, d, J=16.6), 3.20 (1H, dd, J=16.6), 2.50-2.42 (1H, m), 2.25 (2H, qd, J=13.7, 7.4), 2.15 (1H, d, J=11.3), 2.06-2.01 (2H, m), 1.83-1.68 (5H, m), 1.66-1.60 (1H, m), 1.46-1.31 (4H, m), 0.96 (1H, tdd, J=12.4, 5.7, 3.5). ¹³C-NMR (500 MHz, CDCl₃): δ 210.9, 140.0, 135.4, 119.7, 117.7, 114.8, 64.7, 64.3, 51.8, 51.1, 48.2, 45.0, 43.6, 34.7, 32.0, 29.6, 28.4, 23.0, 21.1. IR (neat): ν 3075, 2928, 2857, 1714 cm⁻¹. HRMS (ESI+): calculated for C₁₉H₂₉O₃ ([M+H]⁺) 305.21167, found 305.2113.

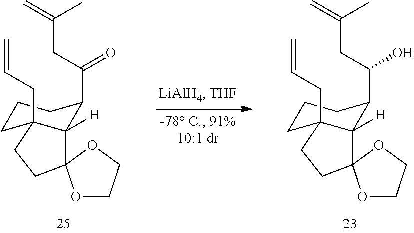

(S)-1-((3a′S,7′R,7a′S)-3a′-allyloctahydrospiro[[1,3]dioxolane-2,1′-indene]-7′-yl)-3-methylbut-3-en-1-ol (23): To a solution of 25 (80 mg, 0.27 mmol, 1 eq.) in 5 mL THF was added lithium aluminum hydride (LAH, 1.0 M in ether, 8 mL, 8 mmol, 30 eq.) at −78° C., and the solution was stirred at −78° C. for 2 hours. The reaction was carefully quenched with Rochelle's salt at −78° C. and slowly warmed to room temperature. The mixture was extracted with ether, and the combined organic layers were washed with saturated NaHCO₃ and brine, then dried over MgSO₄ before removal of the solvent under reduced pressure. The residue was purified by chromatography on silica gel (ethyl acetate/hexane:1/7) to give 23 (73 mg, 91%, 10:1 dr).

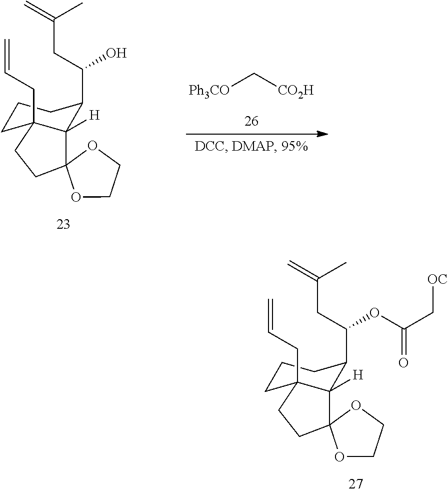

(S)-1-((3a′S,7′R,7a′S)-3a′-allyloctahydrospiro[[1,3]dioxolane-2,1′-indene]-7′-yl)-3-methylbut-3-enyl 2-(trityloxy)acetate (27): To a solution of 23 (53 mg, 0.17 mmol, 1 eq.) in 5 mL dichloromethane was added 4-dimethylaminopyridine (DMAP, 0.10 g, 0.85 mmol, 5 eq), N,N′-dicyclohexylcarbodiimide (DCC, 0.10 g, 0.50 mmol, 3 eq), and acid 264 (0.11 g, 0.35 mmol, 2 eq.) successively at room temperature. The mixture was stirred for 5 hours before quenching with 10 mL saturated aqueous NaHCO₃. The mixture was filtered through Celite and then extracted with ether. The combined organic layers were dried over MgSO₄ before removal of the solvent under reduced pressure. The residue was purified by chromatography on silica gel (ethyl acetate/hexane:7/93) to give 27 (100 mg, 95%). ¹H-NMR (500 MHz, CDCl₃): δ 7.48 (6H, d, J=7.4), 7.33-7.22 (9H, m), 5.80 (1H, td, J=17.5, 7.4), 5.46 (1H, ddd, J=10.3, 4.3, 2.0), 5.08-5.00 (2H, m), 4.71 (1H, s), 4.63 (1H, s), 4.12 (1H, dd, J=13.5, 6.7), 4.05 (1H, dd, J=12.5, 6.9), 3.92 (1H, dd, J=12.6, 7.1), 3.84 (1H, dd, J=13.7, 6.9), 3.70 (2H, q, J=15.4), 2.25-1.99 (5H, m), 1.76-1.62 (7H, m), 1.61-1.15 (7H, m). ¹³C-NMR (500 MHz, CDCl₃): δ 169.4, 143.6, 142.6, 135.6, 128.7, 128.1, 127.3, 118.7, 117.7, 113.1, 87.3, 72.9, 64.8, 64.0, 62.9, 50.0, 45.3, 41.3, 38.1, 35.5, 34.6, 33.8, 30.8, 22.2, 20.0, 17.6. IR (neat): ν 3060, 3024, 2929, 2870, 1754, 1730, 1491, 1448 cm⁻¹. HRMS (ESI+): calculated for C₄₀H₄₆NaO₅ ([M+Na]±) 629.32429, found 629.32415.

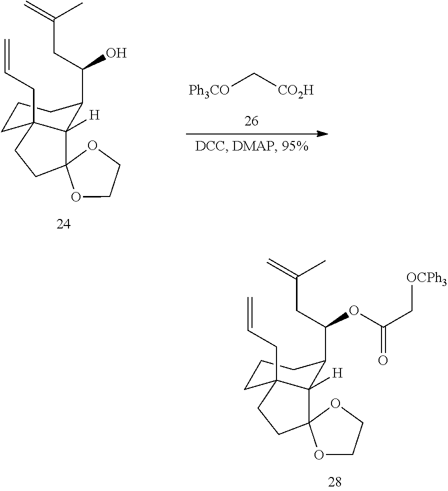

(R)-1-((3a′S,7′R,7a′S)-3a′-allyloctahydrospiro[[1,3]dioxolane-2,1′-indene]-7′-yl)-3-methylbut-3-enyl 2-(trityloxy)acetate (28): To a solution of 24 (50 mg, 0.16 mmol, 1 eq.) in 5 mL dichloromethane was added 4-dimethylaminopyridine (DMAP, 0.10 g, 0.80 mmol, 5 eq), N,N′-dicyclohexylcarbodiimide (DCC, 0.10 g, 0.50 mmol, 3 eq), and acid 264 (0.11 g, 0.35 mmol, 2 eq.) successively at room temperature. The mixture was stirred for 5 hours before quenching with 10 mL saturated aqueous NaHCO₃. The mixture was filtered through Celite and then extracted with ether. The combined organic layers were dried over MgSO₄ before removal of the solvent under reduced pressure. The residue was purified by chromatography on silica gel (ethyl acetate/hexane:7/93) to give 28 (94 mg, 95%). ¹H-NMR (500 MHz, CDCl₃): δ 7.51-7.47 (6H, m), 7.33-7.22 (9H, m), 5.75 (1H, ddt, J=17.3, 10.1, 7.3), 5.42 (1H, td, J=6.9, 4.4), 4.97 (2H, ddd, J=14.7, 12.6, 2.3), 4.75-4.73 (1H, m), 4.68 (1H, s), 4.00-3.90 (2H, m), 3.86 (2H, dq, J=12.4, 6.9), 3.72 (2H, s), 2.25 (3H, dd, J=12.0, 7.2), 2.13 (1H, dd, J=13.7, 7.3), 1.85-1.66 (7H, m), 1.64 (1H, d, J=8.0), 1.56-1.49 (2H, m), 1.45-1.26 (5H, m). ¹³C-NMR (500 MHz, CDCl₃): δ 169.9, 143.6, 142.2, 135.6, 128.7, 128.1, 127.3, 119.6, 117.6, 113.4, 87.4, 73.3, 64.7, 63.9, 62.9, 50.3, 45.4, 42.5, 41.5, 36.0, 34.8, 33.2, 31.9, 22.3, 21.6, 18.5. IR (neat): ν 3060, 2935, 1754, 1727, 1492, 1449 cm⁻¹. HRMS (ESI+): calculated for C₄₀H₄₆NaO₅ ([M+Na]⁺) 629.32429, found 629.32418.

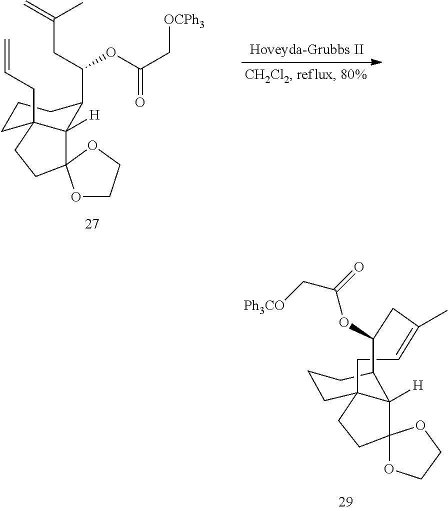

tricyclic pleuromutilin derivative 29: To a solution of 27 (260 mg, 0.43 mmol, 1 eq.) in 300 mL dichloromethane was added Hoveyda-Grubbs catalyst 2nd Generation (Hoveyda-Grubbs II, 30 mg, 0.05 mmol, 0.11 eq.) at room temperature. The solution was refluxed for 24 hours, and another portion of Hoveyda-Grubbs II (30 mg, 0.11 eq.) was added. The solution was refluxed for one week and monitored by TLC. Upon consumption of 27, the solvent was removed under reduced pressure, and the residue was purified by chromatography on silica gel (ethyl acetate/hexane:7/93) to give 29 (200 mg, 80%). ¹H-NMR (500 MHz, CDCl₃): δ 7.50 (6H, d, J=7.5), 7.31 (6H, t, J=7.6), 7.24 (3H, t, J=7.3), 5.43 (1H, t, J=7.8), 4.69 (1H, dd, J=10.6, 5.4), 3.94-3.66 (6H, m), 2.85 (1H, t, J=12.3), 2.48 (1H, dd, J=14.0, 8.6), 2.09 (1H, t, J=6.3), 1.90 (1H, s), 1.88 (3H, s), 1.85-1.61 (7H, m), 1.54 (3H, m), 1.36-1.29 (1H, m), 1.17 (1H, d, J=12.6). ¹³C-NMR (500 MHz, CDCl₃): δ 169.2, 143.5, 137.8, 128.8, 128.1, 127.3, 123.5, 119.4, 87.4, 75.4, 65.1, 63.4, 63.0, 47.3, 42.9, 36.3, 35.4, 35.3, 34.2, 33.4, 31.5, 24.1, 20.0, 19.7. IR (neat): ν 2931, 2869, 1754, 1449, 1203, 1113, 1030 cm⁻¹. HRMS (ESI+): calculated for C₃₈H₄₂NaO₅ ([M+Na]⁺) 601.29299, found 601.29247.

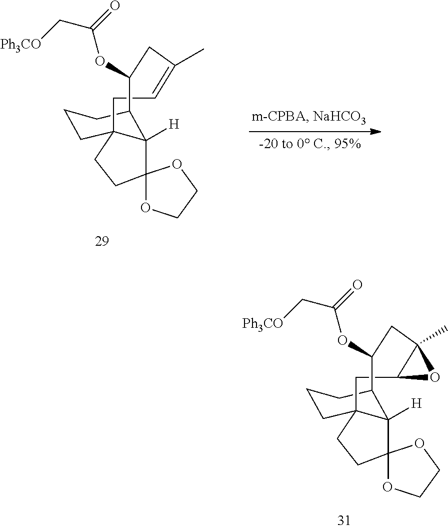

tricyclic pleuromutilin derivative 31: To a solution of 29 (35 mg, 0.061 mmol, 1 eq.) in 5 mL dichloromethane was added NaHCO₃ (20 mg, 0.24 mmol, 4.0 eq.). The suspension was cooled to −20° C. and 3-chloroperoxybenzoic acid (77% m-CPBA, 21 mg, 0.094 mmol, 1.5 eq.) in 2 mL dichloromethane was added dropwise. The mixture was slowly warmed to 0° C. and stirred for 1 hour. Then the reaction was cooled back to −20° C. before addition of another portion of m-CPBA (15 mg, 0.087 mmol, 1.4 eq.) in 1 mL dichloromethane. The mixture was slowly warmed to 0° C. and stirred for 1 hour before saturated aqueous NaHCO₃ was added. The mixture was extracted with ether, and the combined organic layers were washed with saturated aqueous NaHCO₃ and dried over MgSO₄ before removal of the solvent under reduced pressure. The residue was purified by chromatography on silica gel (ethyl/hexane:15/85) to give epoxide 31 (34 mg, 95%). ¹H-NMR (500 MHz, CDCl₃): δ 7.50 (611, d, J=7.5), 7.33 (611, t, J=7.5), 7.27 (3H, dd, J=8.2, 6.0), 4.95 (1H, s), 3.99-3.86 (3H, m), 3.81 (2H, s), 3.79-3.72 (1H, m), 2.81 (1H, dd, J=9.8, 4.7), 2.17 (1H, s), 1.96 (1H, dd, J=14.8, 4.6), 1.93-1.69 (7H, m), 1.61-1.40 (9H, m), 1.11 (1H, d, J=12.6). ¹³C-NMR (500 MHz, CDCl₃): δ 169.2, 143.3, 128.6, 128.0, 127.3, 119.2, 87.4, 74.5, 65.2, 63.4, 62.8, 60.5, 58.7, 48.0, 40.0, 36.7, 36.4, 36.0, 35.0, 32.1, 31.4, 22.3, 19.4, 19.2. M.P.: 201-202° C. IR (neat): ν 2935, 2870, 1754, 1448, 1202, 1112, 1030 cm⁻¹. HRMS (ESI+): calculated for C₃₈H₄₂NaO₆([M+Na]⁺) 617.28791, found 617.28752.

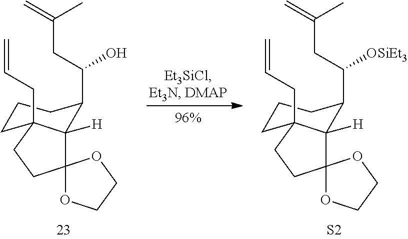

((S)-1-((3a′S,7′R,7a′S)-3a′-allyloctahydrospiro[[1,3]dioxolane-2,1′-indene]-7′-yl)-3-methylbut-3-enyloxy)triethylsilane (S2): To a solution of 23 (40 mg, 0.13 mmol, 1.0 eq.) in 5 mL dichloromethane, triethylamine (Et₃N, 0.05 mL, 36 mg, 0.36 mmol, 2.8 eq.) and 4-dimethylaminopyridine (DMAP, 1.5 mg, 0.012 mmol, 0.1 eq.) were added before addition of triethylsilyl chloride (TESCl, 0.035 mL, 30 mg, 0.20 mmol, 1.5 eq.) dropwise at room temperature. The solution was stirred overnight before addition of another portion of Et₃N (0.05 mL, 36 mg, 0.36 mmol, 2.8 eq.) and TESCl (0.05 mL, 45 mg, 0.30 mmol, 2.3 eq.). The solution was stirred for 3 hours, and Et₃N (0.5 mL, 0.36 g, 3.6 mmol, 28 eq.) was added. Upon consumption of 23, saturated aqueous NaHCO₃ was added. The mixture was extracted with ether, and the combined organic layers were washed with saturated aqueous NaHCO₃ and dried over MgSO₄ before removal of the solvent under reduced pressure. The residue was purified by chromatography on silica gel (ethyl/hexane:5/95) to give silyl ether S2. (53 mg, 96%)¹H-NMR (500 MHz, CDCl₃): δ 5.82 (1H, td, J=16.5, 7.5), 5.06 (2H, d, J=12.3), 4.72 (2H, d, J=13.0), 4.01 (2H, dd, J=15.7, 9.7), 3.96-3.81 (3H, m), 2.21-2.06 (3H, m), 1.98 (1H, t, J=11.6), 1.90 (2H, dd, J=13.0, 9.7), 1.76-1.68 (4H, m), 1.67-1.59 (2H, m), 1.56-1.49 (2H, m), 1.48-1.33 (3H, m), 1.33-1.24 (1H, m), 1.03 (1H, ddd, J=18.1, 12.7, 9.0), 0.93 (9H, t, J=7.9), 0.57 (6H, q, J=7.7). ¹³C-NMR (500 MHz, CDCl₃): δ 144.4, 136.0, 119.1, 117.5, 112.3, 72.0, 64.0, 63.4, 50.2, 45.6, 41.9, 39.7, 38.7, 34.3, 33.2, 31.4, 23.0, 19.1, 18.1, 7.1, 5.1. IR (neat): ν 2954, 2912, 2876, 1639, 1458, 1074, 1005 cm⁻¹. HRMS (ESI+): calculated for C₂₅H₄₅O₃Si ([M+H]±) 421.31380, found 421.31331.

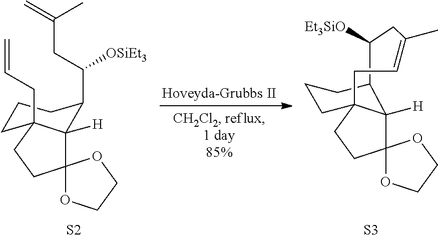

tricyclic pleuromutilin derivative S3: A solution of diene S2 (53 mg, 0.13 mmol. 1.0 eq.) and Hoveyda-Grubbs II (12 mg, 0.018 mmol, 0.14 eq.) in 100 mL dichloromethane was refluxed for 24 hours. Saturated aqueous NaHCO₃ was added, and the mixture was extracted with ether. The combined organic layers were washed with saturated aqueous NaHCO₃ and dried over MgSO₄ before removal of the solvent under reduced pressure. The residue was purified by chromatography on silica gel (ethyl/hexane:5/95) to give S3 (42 mg, 85%). ¹H-NMR (500 MHz, CDCl₃): δ 5.37 (1H, t, J=7.9), 3.97-3.89 (1H, m), 3.85 (2H, dq, J=11.9, 6.1), 3.77-3.68 (1H, m), 3.55 (1H, dd, J=9.9, 5.4), 2.88 (1H, t, J=12.2), 2.51 (1H, dd, J=14.0, 8.5), 2.05-1.91 (2H, m), 1.89-1.78 (6H, m), 1.77-1.62 (4H, m), 1.55-1.39 (3H, m), 1.35-1.28 (1H, m), 1.16 (1H, d, J=12.8), 0.96 (9H, t, J=7.9), 0.66-0.53 (6H, m). ¹³C-NMR (500 MHz, CDCl₃): δ 138.7, 122.8, 120.0, 73.1, 65.3, 63.6, 47.9, 42.9, 39.7, 36.8, 36.3, 35.5, 34.4, 32.0, 24.6, 20.4, 18.6, 7.1, 4.9. IR (neat): ν 2934, 2874, 1734, 1474, 1458, 1071, 1034 cm⁻¹. HRMS (ESI+): calculated for C₂₃H₄₁O₃Si ([M+H]⁺) 393.28250, found 393.28175.

### REFERENCES

1. A. M. Islam, R. A. Raphael J. Chem. S ° C. 1953, 2247.
2. S. A. Bal, A. Marfat, P. Helquist J. Org. Chem. 1982, 47, 5045.
3. S. P. Moore, S.C. Coote, P. O'Brien, J. Gilday Org. Lett. 2006, 8, 5145.
4. H. Auterhoff, R. Oettmeier Archiv der Pharmazie 1975, 308, 732.

### Example 8

General Methods. All reactions were carried out under an atmosphere of argon with magnetic stirring unless otherwise indicated. Palladium (II) acetate was purchased from Gelest. In other cases, commercial reagents of high purity were purchased from either Aldrich or Acros and used without further purification. Tetrahydrofuran (THF), dichloromethane (CH₂Cl₂), toluene, benzene, ether (Et₂O), acetonitrile (CH₃CN), triethylamine (NEt₃), and pyridine were dried by passing through activated alumina columns. Reactions were monitored by thin layer chromatography (TLC) carried out on 0.25 mm Whatman silica gel plates Partisil K6F (60 Å) using UV light as a visualizing agent and aqueous potassium permanganate or ethanolic p-anisaldehyde solution and heat as developing agents. Silica gel from SiliCycle silicaFlash P60 40-63 μm (230-400 mecsh) or from Dynamic Adsorbent Inc 32-63 μm was used for flash column chromatography.

Instrumentation. FT-IR spectra were obtained on a Perkin-Elmer Paragon 500 or a NICOLET 6700. Nuclear magnetic resonance (NMR) spectra were obtained on a 500 MHz Bruker AVANCE spectrometer and calibrated to the residual solvent peak. Coupling constant values were extracted assuming first-order coupling and are given in Hz. The multiplicities are abbreviated as follows: s=singlet, d=doublet, t=triplet, q=quartet, m=multiplet, and br=broad signal. High resolution mass spectra were obtained on a Kratos MS 50 using electrospray ionization (ESI).

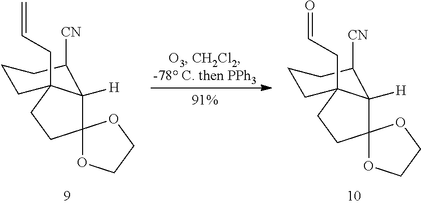

(3a′S, 7′S, 7a′S)-3a′-(2-oxoethyl)octahydrospiro[[1,3]dioxolane-2,1′-indene]-7′-carbonitrile (10): a stream of ozone was bubbled into a solution of 9¹ (871 mg, 3.53 mmol) in dichloromethane (30 ml) at −78° C. Upon consumption of 9 by TLC analysis, a stream of O₂ was bubbled into the reaction for 5 minutes at −78° C. before adding triphenylphosphine (1.39 g, 5.31 mmol). The reaction was allowed to warm to rt and stirred for 3 hours. Evaporation of the dichloromethane and FCC using 40% ethyl acetate in hexanes with 1% triethylamine gave 10 as a colorless oil (791 mg, 91%). ¹H-NMR (500 MHz, CDCl₃): δ 9.74 (1H, t, J=2.1 Hz), 3.95-3.75 (4H, m), 2.86 (1H, dd, J=16.5, 2.3 Hz), 2.55 (2H, ddd, J=18.5, 12.0, 3.3 Hz), 2.08 (1H, d, J=7.2 Hz), 1.98 (3H, dt, J=16.1, 6.1 Hz), 1.75 (1H, dt, J=13.4, 8.3 Hz), 1.67-1.41 (6H, m). ¹³C-NMR (125 MHz, CDCl₃): δ 200.98, 122.26, 116.54, 64.01, 63.32, 51.31, 50.94, 40.80, 33.30, 30.82, 30.21, 26.12, 24.65, 17.83. IR (neat): ν 2943, 2874, 2236, 1720 cm⁻¹. HRMS (ESI+): calculated for C₁₄H₁₉NNaO₃ ([M+Na]⁺) 272.12626, found 272.1271.

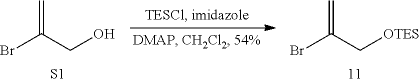

(2-bromoallyloxy)triethylsilane (11): to a solution of 2-bromoallyl alcohol (1.10 g, 8.09 mmol) in dichloromethane (25 ml) was added triethylsilylchloride (TESCl, 1.51 g, 10.11 mmol), imidazole (1.09 g, 16.18 mmol), and 4-dimethylaminopyridine (DMAP, 200 mg, 1.62 mmol) at 0° C. The reaction stirred at rt and was monitored by TLC. Upon consumption of the 2-bromoallyl alcohol, water (10 ml) was added and the organic layer was separated, dried using Na₂SO₄ and evaporated. FCC using pentane gave 11 as a colorless oil (1.10 g, 54%). ¹H-NMR (500 MHz, CDCl₃): δ 5.91 (1H, d, J=1.7 Hz), 5.47 (1H, d, J=1.6 Hz), 4.15 (2H, t, J=1.6 Hz), 0.90 (9H, t, J=8.0 Hz), 0.57 (6H, q, J=8.0 Hz). ¹³C-NMR (125 MHz, CDCl₃): δ 131.71, 114.70, 67.14, 6.73, 4.38. IR (neat): ν 2921, 2878, 1642, 1461 cm⁻¹. HRMS (EI+) calculated for C₃H₄BrO ([M-C₆H₁₅Si]⁺): 134.9446, found 135.

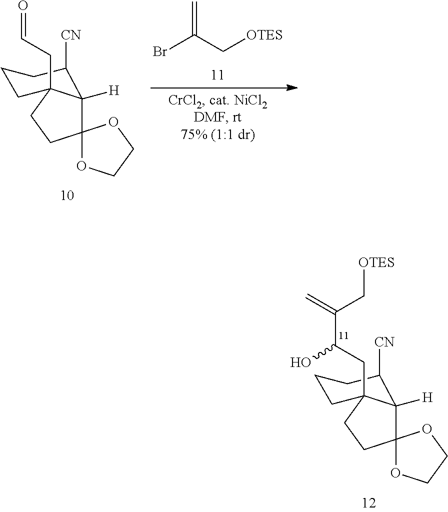

(3a′S, 7′S, 7a′S)-3a′-(2-hydroxy-3-((triethylsilyloxy)methyl)but-3enyl)octahydrospiro[[1,3]dioxolane-2,1′-indene]-7′-carbonitrile (12): to a solution of chromium (II) chloride (1.22 g, 10.03 mmol) and nickel (II) chloride (52 mg, 0.40 mmol) in degassed DMF (40) was added a solution of 10 (500 mg, 2.01 mmol) and 11 (1.10 g, 4.40 mmol) in degassed DMF (10 ml) at rt. The reaction stirred at rt and was monitored by TLC. Upon consumption of 10, a 1.0 M solution of sodium serinate (50 ml) and ethyl acetate (50 ml) were added at 0° C. and the reaction stirred at rt for 1 hour. The aqueous layer was separated and extracted with ethyl acetate (3×25 ml). The organic layers were combined, washed with water (2×25 ml), dried using MgSO₄ and evaporated. FCC using 15% ethyl acetate in hexane gave a partially separable mixture of C11 epimers (12) as a colorless oil (632 mg, 1:1 dr, 75%). Less polar C-11 epimer: ¹H-NMR (500 MHz, CDCl₃): δ 5.02 (1H, s), 4.99 (1H, d, J=1.3 Hz), 4.31-4.21 (2H, m), 4.15 (1H, d, J=13.2 Hz), 3.97-3.79 (4H, m), 2.91 (1H, s), 2.51 (1H, td, J=8.8, 4.4 Hz), 2.12 (1H, d, J=8.1 Hz), 2.07-1.88 (4H, m), 1.75-1.48 (5H, m), 1.46-1.30 (3H, m), 0.91 (9H, t, J=7.9 Hz), 0.57 (6H, q, J=8.0 Hz). ¹³C-NMR (125 MHz, CDCl3): 8150.96, 123.61, 110.37, 71.22, 65.16, 64.37, 64.04, 51.88, 46.11, 43.05, 34.67, 32.01, 31.24, 27.60, 26.04, 19.12, 6.84, 4.35. IR (neat): ν 3503, 2923, 2853, 1457 cm⁻¹. HRMS (ESI+) calculated for C₂₃H₄₀NO₄Si ([M+H]⁺): 422.27266, found 422.2725.

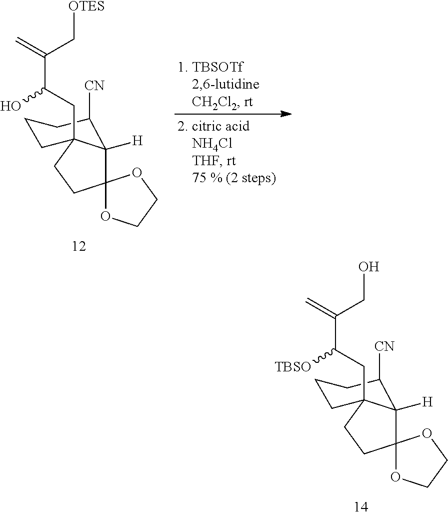

(3a′S, 7′S, 7a′S)-3a′-(2-(tert-butyldimethylsilyloxy)-3-(hydroxymethyl)but-3-enyl)octahydrospiro[[1,3]dioxolane-2,1′-indene]-7′-carbonitrile (14): to a solution of 12 (less polar C-11 epimer) (235 mg, 0.56 mmol) in dichloromethane (10 ml) was added t-butyldimethylsilyl trifluoromethanesulfonate (0.14 ml, 0.61 mmol), 2,6-lutidine (0.10 ml, 0.84 mmol) at 0° C. The reaction stirred at rt and was monitored by TLC. Upon consumption of 12, saturated aqueous NaHCO₃ (5 ml) was added and the organic layer was separated, dried using Na₂SO₄ and evaporated. The crude product was dissolved in THF (8 ml) and a 1.0 M solution of citric acid (1.8 ml) and saturated aqueous NH₄Cl (1.8 ml) were added at 0° C. The reaction stirred at 0° C. for 6 hours and was warmed to rt at which point stirring continued for an additional 3 hours. A saturated aqueous solution of NaHCO₃ (5 ml) and ether (10 ml) were added at 0° C. The aqueous layer was separated and extracted with ether (2×5 ml). The organic layers were combined, dried using MgSO₄, and evaporated. FCC using 30% ethyl acetate in hexane gave 14 as a colorless oil (167 mg, 71% from 12). ¹H-NMR (500 MHz, CDCl₃): δ 5.04 (2H, s), 4.40 (1H, t, J=5.5 Hz), 4.28 (1H, dd, J=14.2, 4.4 Hz), 4.17 (1H, dd, J=14.1, 6.6 Hz), 4.00-3.61 (4H, m), 2.67 (1H, d, J=4.9 Hz), 2.11 (2H, dd, J=14.1, 7.5 Hz), 2.07-1.78 (4H, m), 1.77-1.33 (8H, m), 0.85 (9H, s), 0.07 (3H, s), 0.00 (3H, s). ¹³C-NMR (125 MHz, CDCl₃): δ 151.67, 123.72, 117.76, 111.60, 73.64, 65.04, 63.97, 62.95, 52.49, 45.39, 42.14, 34.66, 32.98, 30.25, 26.42, 25.86, 24.80, 18.39, 17.97, −4.13, −4.81. IR (neat): ν 3459, 2929, 2854, 2240, 1461, 1254 cm⁻¹. HRMS (ESI+) calculated for C₂₃H₄₀NO₄Si ([M+H]⁺): 422.2648, found 422.2722.

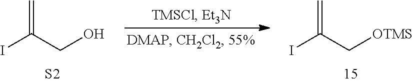

(2-iodoallyloxy)trimethylsilane (15): to a solution of 2-iodoallyl alcohol² (10.00 g, 54.34 mmol) in dichloromethane (100 ml) was added trimethylsilylchloride (13.03 g, 107.98 mmol), triethylamine (23.00 ml, 165.32 mmol), and 4-dimethylaminopyridine (1.32 g, 10.80 mmol) at 0° C. The reaction stirred at rt and was monitored by TLC. Upon consumption of the 2-iodoallyl alcohol, water (50 ml) was added and the organic layer was separated, dried using MgSO₄ and evaporated. Distillation (70° C. @ 10 torr) provided 15 as a pale orange oil (7.70 g, 55%). ¹H-NMR (500 MHz, CDCl₃): δ 6.25 (1H, d, J=1.6 Hz), 5.66 (1H, d, J=1.6 Hz), 4.00 (2H, s), 0.00 (9H, s). ¹³C-NMR (125 MHz, CDCl₃): δ 123.77, 110.03, 71.14, 0.00. IR (neat): ν 2957, 2850, 1626, 1452 cm⁻¹. HRMS (EI+) calculated for C₃H₄₁NaO ([M-C₃H₉Si+Na]⁺): 205.9205, found 206.

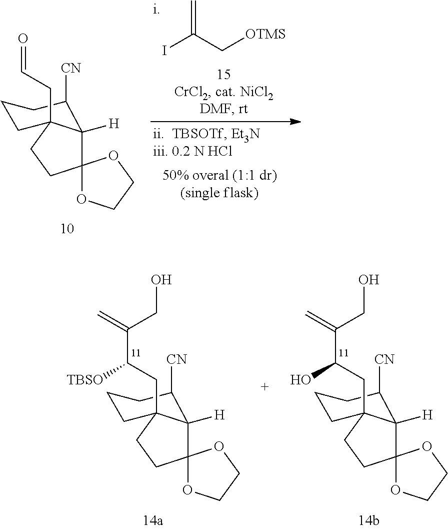

(3a′S, 7′S, 7a′S)-3a′-((S)-2-(tert-butyldimethylsilyloxy)-3-(hydroxymethyl)but-3-enyl)octahydrospiro[[1,3]dioxolane-2,1′-indene]-7′-carbonitrile (14a) & (3a′S, 7′S, 7a′S)-3a′-((R)-2-(tert-butyldimethylsilyloxy)-3-(hydroxymethyl)but-3-enyl)octahydrospiro[[1,3]dioxolane-2,1′-indene]-7′-carbonitrile (14b): to a solution of chromium (II) chloride (3.53 g, 28.92 mmol) and nickel (II) chloride (4 mg, 0.03 mmol) in degassed DMF (40) was added a solution of 10 (720 mg, 2.89 mmol) and 15 (2.96 g, 11.56 mmol) in degassed DMF (10 ml) at rt. The reaction stirred at rt and was monitored by TLC. Upon consumption of 10, triethylamine (17.00 ml, 12.34 mmol) and t-butyldimethylsilyl trifluoromethanesulfonate (16.0 ml, 14.20 mmol) were added successively at rt. After stirring for 30 minutes at rt, the silylation was deemed complete by TLC. A 0.2 M solution of HCl (100 ml) and ethyl acetate (100 ml) were then added at rt and the reaction stirred for 15 minutes. The aqueous layer was separated and extracted with ethyl acetate (3×50 ml). The organic layers were combined, dried using Na₂SO₄ and evaporated. FCC using 25% ethyl acetate in hexanes gave a partially separable mixture of 14a and 14b (609 mg, 1:1 dr, 50% from 10). 14a: ¹H-NMR (500 MHz, CDCl₃): δ 5.04 (2H, s), 4.40 (1H, t, J=5.5 Hz), 4.28 (1H, dd, J=14.2, 4.4 Hz), 4.17 (1H, dd, J=14.1, 6.6 Hz), 4.00-3.61 (4H, m), 2.67 (1H, d, J=4.9 Hz), 2.11 (2H, dd, J=14.1, 7.5 Hz), 2.07-1.78 (4H, m), 1.77-1.33 (8H, m), 0.85 (9H, s), 0.07 (3H, s), 0.00 (3H, s). ¹³C-NMR (125 MHz, CDCl₃): δ 151.67, 123.72, 117.76, 111.60, 73.64, 65.04, 63.97, 62.95, 52.49, 45.39, 42.14, 34.66, 32.98, 30.25, 26.42, 25.86, 24.80, 18.39, 17.97, −4.13, −4.81. 14b: ¹H-NMR (500 MHz, CDCl₃): δ 4.98 (2H, s), 4.31 (1H, dd, J=8.1, 3.5 Hz), 4.20 (2H, s), 3.93-3.73 (4H, m), 2.00-1.78 (6H, m), 1.71 (1H, dd, J=14.8, 8.2 Hz), 1.63-1.45 (6H, m), 1.37 (2H, dd, J=9.3, 3.9 Hz), 0.79 (9H, s), 0.00 (3H, s), −0.07 (3H, s). ¹³C-NMR (125 MHz, CDCl₃): δ 152.01, 123.72, 117.82, 111.19, 73.02, 65.02, 64.26, 63.18, 53.07, 46.32, 42.48, 34.74, 32.67, 30.12, 27.23, 25.92, 25.51, 18.80, 17.98, −3.99, −4.59. IR (neat): ν 3459, 2929, 2854, 2240, 1461, 1254 cm⁻¹. HRMS (ESI+) calculated for C₂₃H₄₀NO₄Si ([M+H]₊): 422.27266, found 422.2722.

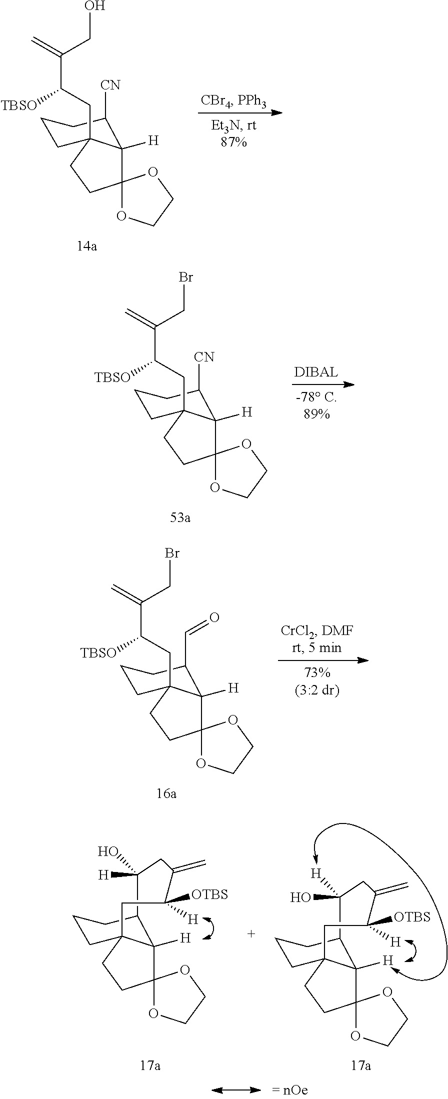

(3a′S,7′R,7a′S)-3a′-((S)-3-(bromomethyl)-2-(tert-butyldimethylsilyloxy)but-3-enyl)octahydrospiro[[1,3]dioxolane-2,1′-indene]-7′-carbonitrile (S3a): to a solution of 14a (19 mg, 0.045 mmol) in dichloromethane (5 ml) was added triethylamine (0.06 ml, 0.45 mmol), triphenylphosphine (60 mg, 0.23 mmol) and carbon tetrabromide (76 mg, 0.23 mmol) successively at rt. The reaction stirred at rt and was monitored by TLC. Upon consumption of 14a, the dichloromethane was evaporated and the crude material was purified by FCC using 10% ethyl acetate in hexanes to give S3a as a colorless oil (19 mg, 87%). ¹H-NMR (500 MHz, CDCl₃): δ 5.31 (1H, s), 5.27 (1H, s), 4.46 (1H, d, J=8.3 Hz), 4.03 (2H, s), 3.98-3.82 (4H, m), 2.77-2.65 (1H, m), 2.17 (1H, ddd, J=14.9, 8.3, 2.4 Hz), 2.03-1.93 (2H, m), 1.88 (2H, dd, J=11.3, 5.2 Hz), 1.82-1.37 (8H, m), 0.89 (9H, s), 0.09 (3H, s), 0.00 (3H, s). ¹³C-NMR (125 MHz, CDCl₃): δ 148.69, 123.65, 117.77, 115.97, 71.50, 65.09, 63.90, 53.48, 46.38, 42.45, 34.75, 33.10, 32.67, 29.82, 26.32, 25.91, 24.76, 18.32, 17.96, −3.88, −4.81. IR (neat): ν 2931, 2859, 2237, 1462, 1257 cm⁻¹. HRMS (ESI+) calculated for C₂₃H₃₉BrNO₃Si ([M+11]⁺): 484.18826, found 484.18764.

(3a′S, 7′R,7a′S)-3a′-((S)-3-(bromomethyl)-2-(tert-butyldimethylsilyloxy)but-3-enyl)octahydrospiro[[1,3]dioxolane-2,1′-indene]-7′-carbaldehyde (16a): to a solution of S3a (19 mg, 0.04 mmol) in toluene (6 ml) was added a 1.0 M solution of diisobutylaluminum hydride in toluene (0.05 ml, 0.05 mmol) dropwise at −78° C. The reaction stirred at −78° C. and was monitored by TLC. Upon consumption of S3a, MeOH (0.5 ml) was added at −78° C. followed and the cold bath was removed. Upon warming to rt, a 1.0 M solution of aqueous citric acid (3 ml) and ether (5 ml) were added. The reaction stirred for an additional 30 minutes at rt and the aqueous layer was separated and extracted with ether (2×5 ml). The organic layers were combined, dried using MgSO₄ and evaporated. FCC using 10% ethyl acetate in hexane with 1% triethylamine gave 16a as a colorless oil (17 mg, 89%). This aldehyde was sensitive to air oxidation upon standing and was therefore used immediately for the next step. ¹H-NMR (500 MHz, CDCl₃): δ 9.60 (1H, d, J=0.8 Hz), 5.20 (1H, s), 5.17 (1H, s), 4.35 (1H, dd, J=7.6, 3.0 Hz), 3.93 (2H, q, J=11.2 Hz), 3.81-3.61 (4H, m), 2.35 (1H, dd, J=12.3, 6.2 Hz), 2.11 (1H, d, J=6.5 Hz), 1.91-1.81 (2H, m), 1.72-1.32 (10H, m), 0.80 (9H, s), 0.00 (3H, s), −0.09 (3H, s). ¹³C-NMR (125 MHz, CDCl₃): δ 204.59, 148.84, 118.64, 115.77, 71.55, 64.69, 64.02, 52.02, 46.69, 46.47, 42.34, 35.02, 32.57, 32.37, 30.48, 25.90, 22.97, 19.19, 17.96, −3.97, −4.81. IR (neat): ν 2930, 2858, 2714, 1722, 1471, 1257, 1090 cm⁻¹. HRMS (ESI+) calculated for C₂₃H₃₉BrNaO₄Si ([M+Na]⁺): 509.16987, found 509.1695.

tricyclic pleuromutilin derivatives 17a & 17b: to a solution of chromium (II) chloride (70 mg, 0.57 mmol) in degassed dimethylformamide (5 ml) was added a solution of 16a (9 mg, 0.019 mmol) in degassed dimethylformamide (1 ml) at rt. The reaction stirred at rt and was monitored by TLC. Upon completion of the reaction (˜5 min), a 1.0 M solution of sodium serinate (5 ml) and ethyl acetate (5 ml) were added at 0° C. The reaction stirred for 15 minutes at rt and the aqueous layer was separated and extracted with ethyl acetate (3×2 ml). The organic layers were combined, dried using Na₂SO₄ and evaporated. FCC using 20% ethyl acetate in hexanes gave a partially separable mixture of epimers 17a and 17b (5.5 mg, 3:2 dr, 73%). 17a: ¹H-NMR (500 MHz, CDCl₃): δ 5.21 (1H, s), 4.98 (1H, s), 4.11 (1H, d, J=8.7 Hz), 3.95-3.84 (3H, m), 3.79-3.65 (2H, m), 2.59 (1H, d, J=14.2 Hz), 2.45 (1H, dd, J=14.2, 8.3 Hz), 2.06 (1H, s), 1.97-1.15 (14H, m), 0.87 (9H, s), 0.03 (3H, s), 0.00 (3H, s). ¹³C-NMR (125 MHz, CDCl₃): δ 150.39, 119.70, 114.26, 74.68, 72.02, 65.06, 63.57, 46.72, 46.52, 39.94, 38.98, 38.49, 36.15, 35.25, 31.84, 25.88, 24.24, 18.13, 17.57, −4.55, −4.74. 17b: ¹H-NMR (500 MHz, CDCl₃): δ 5.07 (1H, s), 5.04 (1H, s), 4.42 (1H, s), 3.98-3.88 (3H, m), 3.87-3.76 (2H, m), 2.79 (1H, t, J=12.4 Hz), 2.16 (2H, dd, J=16.6, 9.2 Hz), 2.02-1.88 (2H, m), 1.86-1.28 (12H, m), 0.88 (9H, s), 0.04 (3H, s), 0.00 (3H, s). ¹³C-NMR (125 MHz, CDCl₃): δ 149.68, 119.66, 115.99, 80.91, 75.52, 64.97, 64.07, 50.52, 46.47, 39.57, 39.49, 34.69, 33.96, 33.78, 33.66, 25.90, 25.80, 19.55, 18.80, 18.11, −4.81, −5.01. IR (neat): ν 3401, 2929, 2870, 1462, 1246, 1038 cm⁻¹. HRMS (ESI+) calculated for C₂₃H₄₁O₄Si ([M+H]₊): 409.27741, found 409.27716.

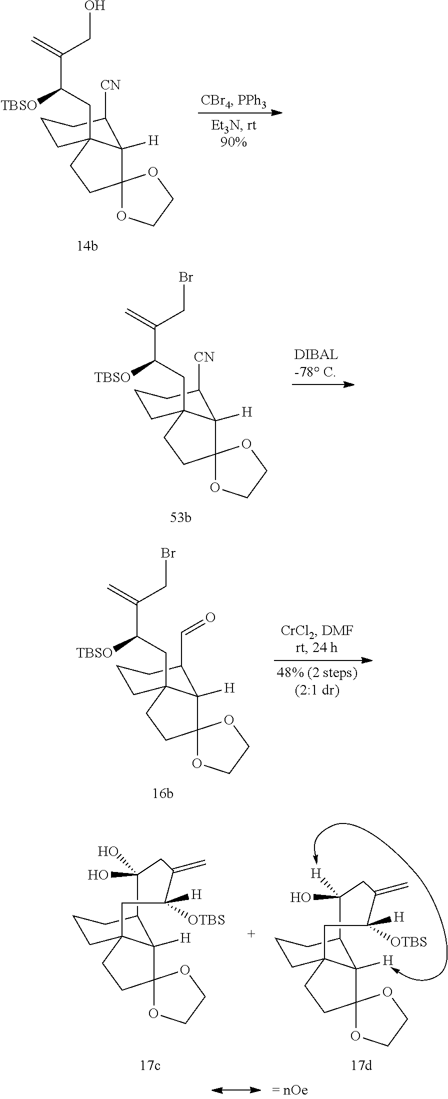

(3a′S,7′R,7a′S)-3a′-((R)-3-(bromomethyl)-2-(tert-butyldimethylsilyloxy)but-3-enyl)octahydrospiro[[1,3]dioxolane-2,1′-indene]-7′-carbonitrile (S3b): to a solution of 14b (150 mg, 0.356 mmol) in dichloromethane (10 ml) was added triethylamine (0.50 ml, 3.56 mmol), triphenylphosphine (466 mg, 1.78 mmol) and carbon tetrabromide (590 mg, 1.78 mmol) successively at rt. The reaction stirred at rt and was monitored by TLC. Upon consumption of 14b, the dichloromethane was evaporated and the crude material was purified by FCC using 10% ethyl acetate in hexanes to give S3b as a colorless oil (156 mg, 90%). ¹H-NMR (500 MHz, CDCl₃): δ 5.25 (1H, s), 5.19 (1H, s), 4.42 (1H, d, J=8.8 Hz), 4.03 (1H, d, J=10.9 Hz), 3.99-3.65 (5H, m), 2.67-2.42 (1H, m), 2.15-1.80 (5H, m), 1.80-1.50 (6H, m), 1.40 (2H, d, J=3.2 Hz), 0.84 (9H, s), 0.05 (3H, s), 0.04 (3H, s). ¹³C-NMR (125 MHz, CDCl₃): δ 149.13, 123.75, 117.89, 115.57, 70.37, 65.02, 64.58, 52.75, 47.07, 42.75, 34.64, 33.35, 32.63, 30.35, 27.58, 26.00, 25.88, 19.03, 17.97, −3.80, −4.48.

(3a′S, 7′R, 7a′S)-3a′-((R)-3-(bromomethyl)-2-(tert-butyldimethylsilyloxy)but-3-enyl)octahydrospiro[[1,3]dioxolane-2,1′-indene]-7′-carbaldehyde (16b): to a solution of S3b (122 mg, 0.253 mmol) in toluene (10 ml) was added a 1.0 M solution of diisobutylaluminum hydride in toluene (0.304 ml, 0.304 mmol) dropwise at −78° C. The reaction stirred at −78° C. and was monitored by TLC. Upon consumption of S3b, MeOH (0.5 ml) was added at −78° C. followed and the cold bath was removed. Upon warming to rt, a 1.0 M solution of aqueous citric acid (5 ml) and ether (5 ml) were added. The reaction stirred for an additional 30 minutes at rt and the aqueous layer was separated and extracted with ether (2×5 ml). The organic layers were combined, dried using MgSO₄ and evaporated to afford the crude aldehyde 16b. This aldehyde was used for the next step without further purification.

tricyclic pleuromutilin derivatives 17c & 17d: in a separate flask, a solution of the crude aldehyde 16b in degassed dimethylformamide (5 ml) was added to a solution of chromium (II) chloride (70 mg, 0.57 mmol) in degassed dimethylformamide (50 ml) at rt. The reaction stirred at rt and was monitored by TLC. After stirring for 24 hours, a 1.0 M solution of sodium serinate (50 ml) and ethyl acetate (50 ml) were added at 0° C. The reaction stirred for 15 minutes at rt and the aqueous layer was separated and extracted with ethyl acetate (3×25 ml). The organic layers were combined, dried using Na₂SO₄ and evaporated. FCC using 15% ethyl acetate in hexanes gave a partially separable mixture of epimers 17c and 17d (49 mg, 2:1 dr, 48% from S3b). 17c: ¹H-NMR (500 MHz, CDCl₃): δ 5.36 (1H, s), 5.10 (1H, s), 4.28 (1H, dd, J=10.2, 3.1 Hz), 3.98-3.87 (3H, m), 3.84-3.76 (1H, m), 3.68 (1H, d, J=3.6 Hz), 2.63 (1H, dd, J=14.2, 6.2 Hz), 2.32 (1H, d, J=14.2 Hz), 2.25-2.11 (2H, m), 1.99 (1H, dt, J=8.8, 4.3 Hz), 1.88 (1H, dd, J=13.4, 3.3 Hz), 1.85-1.37 (9H, m), 1.24-1.13 (2H, m), 0.88 (9H, s), 0.02 (3H, s), 0.00 (3H, s). ¹³C-NMR (125 MHz, CDCl₃): δ 149.27, 119.74, 116.25, 74.04, 73.54, 64.93, 64.23, 42.98, 40.78, 38.49, 38.12, 36.21, 35.43, 33.24, 29.72, 25.89, 25.04, 19.56, 18.35, −4.66, −5.12. 17d: ¹H-NMR (500 MHz, CDCl₃): δ 5.39 (1H, s), 5.08 (1H, t, J=1.5 Hz), 4.26 (1H, dd, J=10.6, 3.9 Hz), 3.99-3.88 (3H, m), 3.86-3.75 (2H, m), 2.52 (1H, dd, J=13.1, 1.9 Hz), 2.17 (2H, ddd, J=19.2, 12.9, 3.4 Hz), 1.95 (1H, dd, J=13.0, 3.9 Hz), 1.90 (1H, s), 1.73-1.39 (12H, m), 0.87 (9H, s), 0.02 (3H, s), 0.00 (3H, s). ¹³C-NMR (125 MHz, CDCl₃): δ 150.77, 119.75, 115.13, 80.46, 73.65, 64.89, 64.30, 57.94, 46.63, 41.04, 39.80, 37.95, 35.49, 34.47, 32.94, 29.72, 25.89, 25.80, 20.20, 19.91, 18.40, −4.66, −5.19. IR (neat): ν 3401, 2929, 2870, 1462, 1246, 1038 cm⁻¹. HRMS (ESI+) calculated for C₂₃H₄₁O₄Si ([M+11]±): 409.27741, found 409.2772.

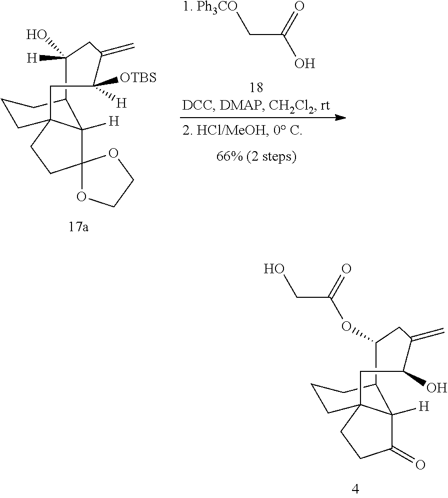

tricyclic pleuromutilin derivative 4: To a solution of 17a (5 mg, 0.022 mmol) and 18 (28 mg, 0.088 mmol) in dichloromethane (2 ml) was added dicyclohexylcarbodiimide (18 mg, 0.088 mmol) and 4-dimethylaminopyridine (1 mg, 0.008 mmol) at rt. The reaction stirred for 1 hour at which point 17a was completely consumed. Water (1 ml) was added to the reaction and the aqueous layer was separated and extracted with dichloromethane (2 ml). The organic layers were combined, dried using Na₂SO₄ and evaporated. FCC using 5% ethyl acetate in hexane gave the desired acylation product with some impurities that were inseparable by FCC. This mixture was taken on directly to the next step without further purification. A 3% solution of HCl in MeOH (3 ml) was added to the mixture at 0° C. The reaction stirred at 0° C. and was monitored by TLC. Upon completion of the reaction (−4 hours), solid NaHCO₃ (400 mg) was added and the reaction stirred for 5 minutes before filtering through a small plug of celite. Evaporation and FCC using 60% ethyl acetate in hexane gave 4 as a clear colorless oil (2.5 mg, 66%, 2 steps). ¹H-NMR (500 MHz, MeOD): δ 5.22 (1H, d, J=1.2 Hz), 5.00 (1H, dt, J=8.7, 5.3 Hz), 4.92 (1H, s), 4.08 (1H, d, J=9.6 Hz), 4.03 (2H, d, J=1.7 Hz), 2.51-2.37 (3H, m), 2.31-2.13 (3H, m), 1.90 (2H, dt, J=14.5, 6.0 Hz), 1.66 (1H, d, J=14.9 Hz), 1.61-1.42 (3H, m), 1.34-1.02 (6H, m). ¹³C-NMR (125 MHz, MeOD): δ 219.79, 174.14, 151.81, 113.72, 77.17, 70.72, 61.25, 54.02, 45.41, 40.54, 39.63, 36.66, 35.08, 33.21, 32.35, 24.34, 17.66. IR (neat): ν 3401, 2926, 1734, 1724, 1428, 1117 cm⁻¹. HRMS (ESI+) calculated for C₁₇H₂₄NaO₅([M+Na]⁺): 331.15214, found 331.1518.

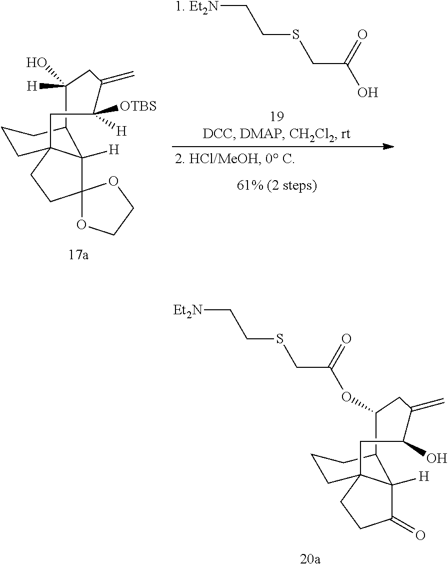

tricyclic pleuromutilin derivative 20a: to a solution of 17a (40 mg, 0.098 mmol) and 19 (28 mg, 0.147 mmol) in dichloromethane (5 ml) was added dicyclohexylcarbodiimide (30 mg, 0.147 mmol) and 4-dimethylaminopyridine (1 mg, 0.008 mmol) at rt. The reaction stirred for 12 hours at which point 17a was completely consumed. Water (3 ml) was added to the reaction and the aqueous layer was separated and extracted with dichloromethane (2 ml). The organic layers were combined, dried using Na₂SO₄ and evaporated. To the crude product was added a 3% solution of HCl in MeOH (4 ml) at 0° C. The reaction stirred at 0° C. and was monitored by TLC. Upon completion of the reaction (˜2 hours), solid NaHCO₃ (400 mg) was added and the reaction stirred for 5 minutes before filtering through a small plug of celite. Evaporation and FCC using 10% MeOH in dichloromethane gave 20a as a clear colorless oil (25 mg, 61% from 17a). ¹HNMR (500 MHz, D₂O): δ 5.15 (1H, s), 4.98 (1H, s), 4.94 (1H, dt, J=8.5, 5.2 Hz), 4.20 (1H, d, J=9.8 Hz), 3.36 (2H, dd, J=8.7, 5.1 Hz), 3.20-3.10 (2H, m), 3.00 (4H, q, J=7.3 Hz), 2.90-2.78 (2H, m), 2.55 (1H, s), 2.47 (2H, d, J=4.9 Hz), 2.31 (2H, dd, J=9.8, 5.5 Hz), 2.23 (1H, s), 1.98-1.82 (2H, m), 1.68 (1H, d, J=14.8 Hz), 1.60-1.40 (2H, m), 1.24-1.05 (6H, m), 1.14 (6H, t, J=7.3 Hz). ¹³C-NMR (125 MHz, D₂O): δ 224.44, 172.53, 149.02, 113.84, 77.43, 69.80, 52.82, 50.40, 47.19, 42.72, 38.24, 37.75, 35.01, 34.29, 33.30, 31.47, 30.70, 26.32, 22.98, 16.06, 8.50.

IR (neat): ν 3391, 2926, 2854, 1732, 1715, 1652, 1456, 1275 cm⁻¹. HRMS (ESI+) calculated for C₂₃H₃₈NO₄S ([M+H]⁺): 424.25215, found 424.25167.

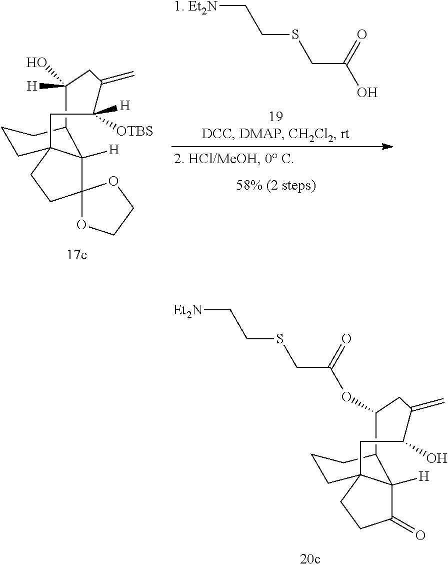

tricyclic pleuromutilin derivative 20c: To a solution of 17c (16 mg, 0.039 mmol) and 19 (9 mg, 0.047 mmol) in dichloromethane (5 mL) was added dicyclohexylcarbodiimide (12 mg, 0.058 mmol) and 4-dimethylaminopyridine (1 mg, 0.008 mmol) at rt. The reaction stirred overnight, and additional portions of 19 (9 mg, 0.047 mmol) and dicyclohexylcarbodiimide (12 mg, 0.058 mmol) were added during this period. After 17c was nearly consumed, water (5 mL) was added to the reaction and the aqueous layer was separated and extracted twice with dichloromethane (5 mL). The organic layers were combined, dried using MgSO₄, and evaporated. FCC using 5% MeOH in dichloromethane gave 22 mg coupling product, which was taken on directly to the next step. A 3% solution of HCl in MeOH (8.25 mL) was added to the mixture at 0° C. and allowed to stir at 0° C. for 1 hr. The reaction was then allowed to warm to room temperature and was complete by TLC after 1.5 hrs. Solid NaHCO₃ was added (100 mg) and the reaction was allowed to stir for 5 minutes before filtering through a small plug of Celite. Evaporation and FCC using 7-10% MeOH in dichloromethane gave 20c as a colorless oil (9.5 mg, 58%, 2 steps). ¹H-NMR (500 MHz, D₂O): δ 5.31 (1H, s), 5.12 (1H, s), 5.05 (1H, t, J=5.7), 4.59 (1H, d, J=8.3), 3.47 (1H, d, J=3.3), 3.38 (2H, t, J=7.4), 3.25 (411, q, J=7.1), 3.01-2.93 (2H, m), 2.74 (1H, dd, J=14.9, 7.1), 2.65 (1H, s), 2.49 (2H, d, J=13.3), 2.36-2.30 (2H, m), 2.07 (1H, d, J=12.3), 1.96-1.83 (2H, m), 1.79-1.72 (1H, m), 1.56 (2H, dd, J=33.0, 14.1), 1.44-1.17 (11H, m). ¹³C-NMR (125 MHz, D₂O): δ 227.15, 171.96, 147.74, 117.30, 77.91, 72.95, 51.29, 50.29, 47.41, 38.82, 36.27, 35.32, 34.46, 33.31, 32.50, 25.93, 23.31, 18.68, 8.00. HRMS (ESI+) calculated for C₂₃H₃₈NO₄S ([M+H]⁺): 424.25215, found 424.25177.

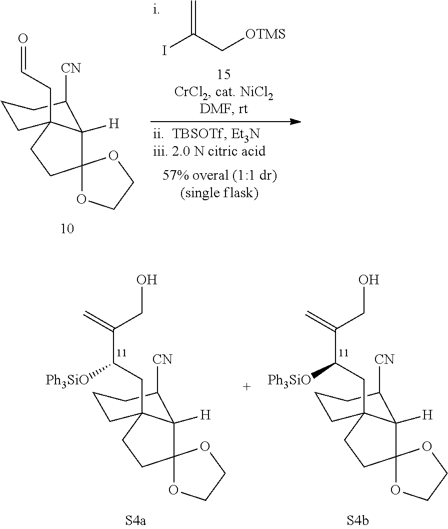

(3a′S, 7′S, 7a′S)-3a′-(3-(hydroxymethyl)-2-(triphenylsilyloxy)but-3-enyl)octahydrospiro[[1,3]dioxolane-2,1′-indene]-7′-carbonitrile (S4): To a solution of chromium (II) chloride (3.48 g, 14.26 mmol) and nickel (II) chloride (3.7 mg, 0.028 mmol) in degassed DMF (30 ml) was added a solution of 10 (710 mg, 2.83 mmol) and 15 (2.90 g, 11.32 mmol) in degassed DMF (15 ml) at rt. The reaction stirred at rt and was monitored by TLC. Upon consumption of 10 (−3 hours), triethylamine (11.84 ml, 84.93 mmol), 4-dimethylaminopyridine (345 mg, 2.83 mmol) and triphenylsilylchloride (16.69 g, 56.62 mmol) in dimethylformamide (30 ml) were added successively at rt. After stirring for 3 hours at rt, the silylation was deemed complete by TLC. A 1.0 N solution of sodium serinate (300 ml) and ethyl acetate (200 ml) were then added at rt and the reaction stirred for 30 minutes. The aqueous layer was separated and extracted with ethyl acetate (2×150 ml). The organic layers were combined and a solution of 2.0 N aqueous citric acid was added at rt. The reaction stirred for ˜1 hour and the organic layer was separated, washed with sat. NaHCO₃, dried using Na₂SO₄ and evaporated. FCC using ethyl acetate in hexanes (15%″ 40%) gave S4 as a colorless oil (908 mg, 1:1 dr, 57% from 10). Desired C-11 epimer S4a: ¹H-NMR (500 MHz, CDCl₃): δ 7.57 (6H, d, J=6.8 Hz), 7.37 (3H, t, J=7.3 Hz), 7.31 (6H, t, J=7.2 Hz), 4.95 (1H, s), 4.83 (1H, s), 4.52 (1H, t, J=6.1 Hz), 4.22 (1H, dd, J=14.3, 4.2 Hz), 4.06 (1H, dd, J=14.3, 7.8 Hz), 4.01-3.71 (4H, m), 2.54 (1H, dd, J=11.4, 5.9 Hz), 2.13 (1H, dd, J=14.7, 5.7 Hz), 1.81 (3H, ddd, J=13.3, 11.1, 5.8 Hz), 1.69 (2H, t, J=7.9 Hz), 1.48-1.12 (8H, m). ¹³C-NMR (125 MHz, CDCl₃): δ 149.36, 134.65, 132.94, 129.07, 126.79, 122.59, 116.43, 112.03, 73.61, 63.94, 62.92, 61.24, 52.09, 43.97, 40.96, 33.59, 31.34, 28.76, 25.41, 23.77, 17.19. IR (neat): ν 3468, 3069, 2929, 2237, 1429, 1116, 1024 cm⁻¹. HRMS (ESI+) calculated for C₃₅H₄₀NO₄Si ([M+H]⁻¹⁻): 566.27266, found 566.2715.

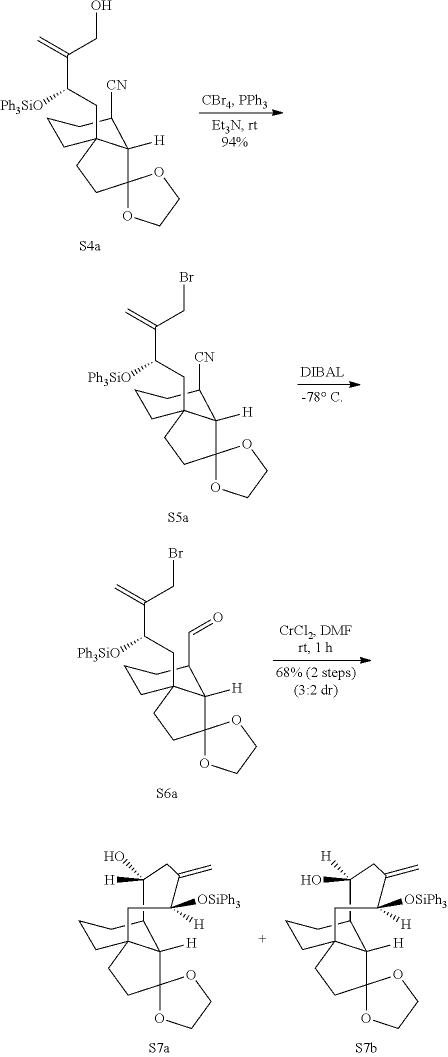

(3a′S, 7′R, 7a′S)-3a′-((S)-3-(bromomethyl)-2-(triphenylsilyloxy)but-3-enyl)octahydrospiro[[1,3]dioxolane-2,1′-indene]-7′-carbonitrile (S5a): to a solution of S4a (140 mg, 0.247 mmol) in dichloromethane (15 ml) was added triethylamine (0.34 ml, 2.47 mmol), triphenylphosphine (324 mg, 1.24 mmol) and carbon tetrabromide (409 mg, 1.24 mmol) successively at rt. The reaction stirred at rt and was monitored by TLC. Upon consumption of S4a, the dichloromethane was evaporated and the crude material was purified by FCC using 20% ethyl acetate in hexanes to give S5a as a colorless oil (145 mg, 94%). ¹H-NMR (500 MHz, CDCl₃): δ 7.56 (6H, d, J=6.8 Hz), 7.33 (9H, dt, J=29.0, 7.2 Hz), 5.12 (1H, s), 5.07 (1H, s), 4.53 (1H, t, J=5.6 Hz), 3.91 (1H, d, J=11.7 Hz), 3.88-3.69 (5H, m), 2.61-2.49 (1H, m), 2.29 (1H, dd, J=14.8, 6.5 Hz), 1.87-1.61 (5H, m), 1.54-1.06 (7H, m). ¹³C-NMR (125 MHz, CDCl₃): 8146.06, 134.68, 132.89, 129.02, 126.76, 122.55, 116.80, 116.48, 72.50, 63.95, 62.88, 52.75, 44.59, 41.24, 33.62, 31.38, 30.64, 28.35, 25.38, 23.80, 17.24. IR (neat): n□2924, 2860, 2236, 1429, 1116, 1024 cm⁻¹. HRMS (ESI+) calculated for C₃₅H₃₉BrNO₃Si ([M+H]⁺): 628.18826, found 628.1793.

(3a′S,7′R,7a′S)-3a′-((9)-3-(bromomethyl)-2-(triphenylsilyloxy)but-3-enyl)octahydrospiro[[1,3]dioxolane-2,1′-indene]-7′-carbaldehyde (S6a): to a solution of S5a (132 mg, 0.210 mmol) in toluene (10 ml) was added a 1.0 M solution of diisobutylaluminum hydride in toluene (0.253 ml, 0.253 mmol) dropwise at −78° C. The reaction stirred at −78° C. and was monitored by TLC. Upon consumption of S5a, MeOH (0.5 ml) was added at −78° C. followed and the cold bath was removed. Upon warming to rt, a 1.0 M solution of aqueous citric acid (5 ml) and ether (5 ml) were added. The reaction stirred for an additional 30 minutes at rt and the aqueous layer was separated and extracted with ether (2×5 ml). The organic layers were combined, dried using MgSO₄ and evaporated to afford the crude aldehyde. This aldehyde S6a was used for the next step without further purification.

tricyclic pleuromutilin derivatives S7a & S7b: to a solution of chromium (II) chloride (385 mg, 3.16 mmol) in degassed dimethylformamide (70 ml) was added a solution of crude aldehyde S6a in degassed dimethylformamide (10 ml) at rt. The reaction stirred at rt and was monitored by TLC. Upon completion of the reaction 1 h), a 1.0 M solution of sodium serinate (50 ml) and ethyl acetate (50 ml) were added at 0° C. The reaction stirred for 30 minutes at rt and the aqueous layer was separated and extracted with ethyl acetate (2×50 ml). The organic layers were combined, dried using Na₂SO₄ and evaporated. FCC using 30% ethyl acetate in hexanes gave a partially separable mixture of epimers S7a and S7b (76 mg, 3:2 dr, 68%). S7a: ¹H-NMR (500 MHz, CDCl₃): δ 7.60-7.49 (6H, m), 7.32 (9H, dt, J=28.6, 7.2 Hz), 5.15 (1H, s), 4.98 (1H, s), 4.28 (1H, d, J=9.3 Hz), 3.85-3.72 (3H, m), 3.71-3.54 (2H, m), 2.57 (1H, d, J=14.2 Hz), 2.34 (1H, dd, J=14.2, 7.7 Hz), 2.05-1.95 (1H, m), 1.91-0.76 (14H, m). ¹³C-NMR (125 MHz, CDCl₃): δ 147.60, 134.53, 133.52, 128.92, 126.74, 118.42, 115.88, 73.29, 72.05, 63.92, 62.43, 44.66, 43.92, 37.46, 37.19, 37.02, 34.14, 34.01, 30.78, 23.36, 16.81. IR (neat): n03396, 2925, 2855, 1429, 1261, 1116, 1024. HRMS (ESI+) calculated for C₃₅H₄₁O₄Si ([M+H]±): 553.27741, found 553.2767.

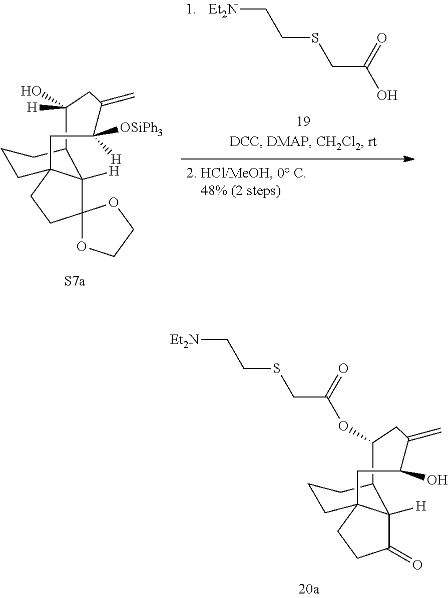

tricyclic pleuromutilin derivative 20a: to a solution of S7a (33 mg, 0.060 mmol) and 19 (24 mg, 0.126 mmol) in dichloromethane (5 ml) was added dicyclohexylcarbodiimide (30 mg, 0.147 mmol) and 4-dimethylaminopyridine (1 mg, 0.008 mmol) at rt. The reaction stirred for 12 hours at which point S7a was completely consumed. Water (3 ml) was added to the reaction and the aqueous layer was separated and extracted with dichloromethane (2 ml). The organic layers were combined, dried using Na₂SO₄ and evaporated. To the crude product was added a 3% solution of HCl in MeOH (4 ml) at 0° C. The reaction stirred at 0° C. and was monitored by TLC. Upon completion of the reaction (˜2 hours), solid NaHCO₃ (500 mg) was added and the reaction stirred for 5 minutes before filtering through a small plug of celite. Evaporation and FCC using 10% MeOH in dichloromethane gave 20a as a clear colorless oil (12 mg, 48% from S7a).

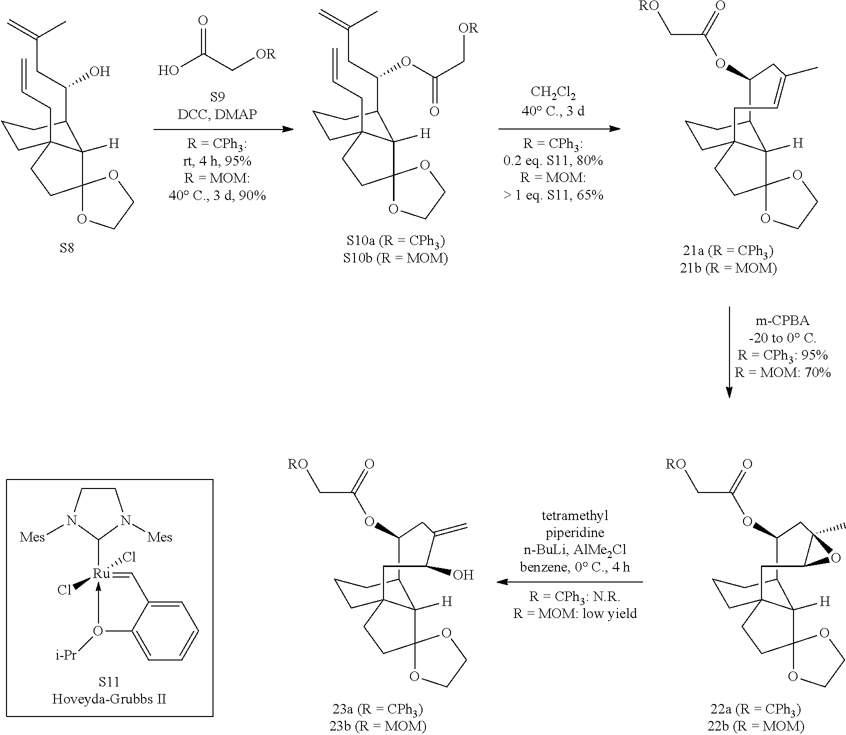

For general experimental procedures, see Ref. 1. (Characterization of S10a, 21a, 22a included).

(S)-1-((3a′S,7R,7a′S)-3a′-allyloctahydrospiro[[1,3]dioxolane-2,1′-indene]-7′-yl)-3-methylbut-3-enyl 2-(methoxymethoxy)acetate (S10b): ¹H-NMR (500 MHz, CDCl₃): δ 5.86-5.73 (1H, m), 5.50-5.46 (1H, m), 5.07 (1H, s), 5.05 (1H, d, J=6.1), 4.76-4.65 (4H, m), 4.16-4.09 (3H, m), 4.05 (1H, dd, J=13.4, 7.4), 3.92 (1H, dd, J=12.9, 7.0), 3.85 (1H, dd, J=13.6, 6.8), 3.38 (3H, s), 2.31-2.01 (5H, m), 1.78-1.18 (14H, m).

¹³CNMR (125 MHz, CDCl₃): δ 169.71, 142.65, 135.62, 118.64, 117.74, 113.18, 96.30, 73.42, 64.75, 64.14, 63.96, 55.83, 49.90, 45.32, 41.28, 38.11, 35.50, 34.66, 33.79, 30.88, 22.11, 20.14, 17.55. IR (neat): ν 2939, 1750, 1441, 1194, 1151, 1120, 1060 cm⁻¹. HRMS (ESI+): calculated for C₂₃H₃₇O₆ ([M+H]⁺) 409.25901, found 409.25834.

tricyclic pleuromutilin derivative 21b: ¹H-NMR (500 MHz, CDCl₃): δ 5.44 (1H, t, J=8.0), 4.76 (1H, dd, J=10.4, 5.5), 4.73 (2H, s), 4.16 (2H, s), 3.93-3.84 (3H, m), 3.74-3.69 (1H, m), 3.41 (3H, s), 2.93 (1H, t, J=12.3), 2.49 (1H, dd, J=14.1, 8.5), 2.14 (1H, t, J=6.3), 1.92 (1H, s), 1.87 (3H, s), 1.85-1.48 (10H, m), 1.36 (1H, s), 1.19 (1H, d, J=13.0). ¹³C-NMR (125 MHz, CDCl₃): δ 169.52, 137.69, 123.66, 119.41, 96.48, 75.76, 65.17, 64.51, 63.46, 55.98, 47.42, 42.94, 36.31, 35.54, 35.30, 34.29, 33.40, 31.54, 24.10, 20.08, 19.68. IR (neat): ν 2933, 1750, 1202, 1151, 1123, 1062, 1029 cm⁻¹. HRMS (ESI+): calculated for C₂₁H₃₃O₆ ([M+H]±) 381.22771, found 381.22758.

tricyclic pleuromutilin derivative 22b: ¹H-NMR (500 MHz, CDCl₃): δ 5.01 (1H, ddd, J=11.3, 5.7, 2.6), 4.73 (2H, s), 4.18 (2H, s), 3.96-3.90 (2H, m), 3.89 (1H, dd, J=11.0, 4.9), 3.78-3.71 (1H, m), 3.41 (3H, s), 2.81 (1H, dd, J=10.2, 5.0), 2.21 (1H, t, J=5.4), 2.00-1.68 (8H, m), 1.61-1.39 (9H, m), 1.11 (1H, d, J=12.8). ¹³C-NMR (125 MHz, CDCl₃): δ 169.46, 119.27, 96.47, 74.97, 65.33, 64.42, 63.58, 60.55, 58.74, 55.98, 48.17, 40.06, 36.83, 36.63, 36.09, 35.10, 32.25, 31.53, 22.40, 19.51, 19.27. IR (neat): ν 2936, 1752, 1202, 1151, 1122, 1062, 1030 cm⁻¹. HRMS (ESI+): calculated for C₂₁H₃₃O₇ ([M+H]⁺) 397.22263, found 397.22267.

tricyclic pleuromutilin derivative 23b: ¹H-NMR (500 MHz, CDCl₃): δ 5.37 (1H, s), 5.34 (1H, s), 4.90 (1H, ddd, J=11.7, 6.1, 3.0), 4.73 (2H, s), 4.32 (1H, d, J=10.3), 4.19 (2H, s), 3.93-3.83 (3H, m), 3.75-3.66 (1H, m), 3.41 (3H, s), 2.68 (1H, t, J=12.4), 2.36 (1H, d, J=11.4), 2.21 (1H, s), 2.10 (1H, dd, J=14.6, 11.2), 1.92 (1H, s), 1.87-1.62 (6H, m), 1.59-1.37 (5H, m), 1.24 (1H, d, J=12.2). ¹³C-NMR (125 MHz, CDCl₃): δ 169.54, 150.58, 120.40, 119.44, 96.46, 82.27, 71.58, 65.23, 64.45, 63.64, 55.96, 45.99, 41.35, 38.51, 36.46, 34.82, 32.34, 32.15, 31.83, 19.43, 19.24. IR (neat): ν 3470, 2937, 1749, 1204, 1151, 1122, 1061 cm⁻¹. HRMS (ESI+): calculated for C₂₁H₃₃O₇ ([M+H]⁺) 397.22263, found 397.22206.

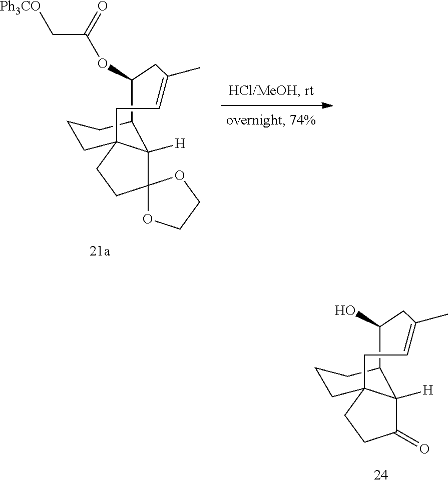

tricyclic pleuromutilin derivative 24: concentrated HCl (0.7 mL) was dissolved in a mixture of 5 mL methanol and 1 ml dichloromethane. To a vial with the substrate 21a1 (50 mg, 0.087 mg) was added this solution. The mixture was stirred overnight and monitored by TLC. Upon complete consumption of 21a, the solvent was removed under reduced pressure, and then ether and water was added. The aqueous layer was separated and extracted with ether and then dichloromethane. The organic layers were combined, dried using Na₂SO₄ and evaporated. The residue was dissolved with a small amount of solvent mixture (ethyl acetate/hexane/dichloromethan) before loaded to the FCC column; FCC using ethyl acetate in hexane (9% 25%) gave 24 (15 mg, 74%). ¹H-NMR (500 MHz, CDCl₃): δ 5.48 (1H, t, J=8.0), 3.66 (1H, dd, J=10.5, 5.7), 2.88 (1H, t, J=12.3), 2.67 (1H, s), 2.54 (1H, dd, J=14.1, 8.6), 2.22 (2H, dt, J=17.2, 8.4), 1.97 (1H, s), 1.94-1.62 (9H, m), 1.59-1.52 (1H, m), 1.43-1.34 (2H, m), 1.33-1.23 (1H, m), 1.16-1.05 (1H, m). ¹³C-NMR (125 MHz, CDCl₃): δ 218.74, 139.12, 122.93, 71.39, 53.82, 43.28, 38.34, 35.75, 35.15, 34.34, 33.05, 32.96, 24.41, 19.79, 18.66. IR (neat): ν 3413, 2924, 2855, 1734, 1474, 1454, 1232, 1171, 1106, 1078, 1060, 1048, 1035, 1018 cm⁻¹. HRMS (ESI+): calculated for C₁₅H₂₃O₂ ([M+H]⁺) 235.16980, found 235.16964.

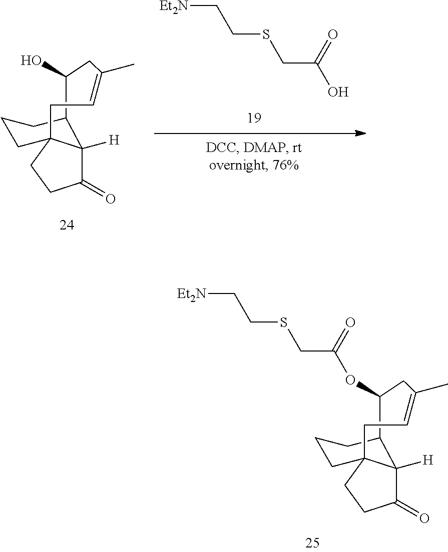

tricyclic pleuromutilin derivative 25: alcohol 24 (6 mg, 0.026 mmol) and acid 19 (15 mg, 0.077 mmol) were dissolved in 3 mL dichloromethane. To this mixture was added N,N′-Dicyclohexylcarbodiimide (DCC, 16 mg, 0.077 mmol) and 4-Dimethylaminopyridine (DMAP, 0.3 mg, 0.0026 mmol) at room temperature and then stirred overnight. The mixture was filtered through Celite to remove insoluble impurities. FCC using 6% methanol in dichloromethane multiple times gave pure 25 (7.9 mg, 76%). ¹H-NMR (500 MHz, CDCl₃): δ 5.53 (1H, t, J=8.0), 4.73 (1H, dd, J=11.8, 5.8), 3.26 (2H, s), 2.96 (1H, t, J=12.4), 2.75 (5H, bs), 2.57 (5H, dt, J=14.4, 7.7), 2.31-2.17 (2H, m), 2.00 (1H, s), 1.95-1.76 (7H, m), 1.70 (1H, dd, J=23.0, 10.4), 1.58 (1H, dd, J=11.5, 9.4), 1.49-1.36 (2H, m), 1.32-1.23 (1H, m), 1.21-1.09 (1H, m), 1.05 (6H, t, J=7.1). ¹³C-NMR (125 MHz, CDCl₃): δ 217.39, 169.65, 138.45, 123.53, 74.14, 53.62, 52.26, 47.04, 43.17, 35.55, 34.99, 34.33, 34.19, 32.97, 32.85, 32.84, 30.02, 23.95, 19.82, 19.66, 11.77. IR (neat): ν 2964, 2925, 2850, 1736, 1455, 1382, 1267, 1105 cm⁻¹. HRMS (ESI+): calculated for C₂₃H₃₈NO₃S ([M+H]⁺) 408.25724, found 408.25779.

Determination of MIC values for pleuromutilin family members versus M. tuberculosis mc27000. A drug susceptibility assay in 96-well plate format by Alamar Blue (resazurin) viability assay was modified from Franzblau and co-workers.³ The bacteria were grown to mid-log phase in ADC supplemented 7H9 media (Middlebrook) (OD₆₀₀=0.5) and diluted to OD₆₀₀=0.003 into 7H9 media containing 0.05% tyloxapol, 0.2% dextrose, and 25 ng/mL pantothenate. 196 μl of diluted culture was dispensed into each well of a sterile 96-well plate. Compounds were dissolved in DMSO and subsequent ½ serial dilutions were performed in DMSO. 4 μL of serial diluted drug solutions were added to testing wells, while the outer perimeter wells were injected with the same volume of DMSO for a negative control. The plates were sealed with film and were incubated at 37° C. for 5 days with shaking. Resazurin stock solution was added to every well to a final concentration of 5 μg/mL. The plates were reincubated at 37° C. for 24 h, and the colors of all wells were recorded. A blue color in the well was interpreted as no respiration, and a pink color was scored as viable cells, reducing resazurin to resorufin coupled to respiration. Wells appearing as violet after 24 h of incubation would invariably change to pink after longer incubation and thus were scored as growth (while the adjacent blue wells remained blue). The MIC was recorded as the lowest drug concentration that prevented a color change from blue to pink.

### REFERENCES

1. J. Liu, S. D. Lotesta and E. J. Sorensen, Chem. Commun., 2011, 47, 1500.
2. M. Kurosu, M. Lin, Y. Kishi, J. Am. Chem. Soc., 2004, 126, 12248.
3. L. A. Collins and S. Franzblau, Antimicrob. Agents Chemother., 1997, 41, 1004.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A method of synthesizing a pleuromutilin analog comprising providing a pleuromutilin skeleton having the structure of one of formulas 6-1, 6-2 or 6-3: 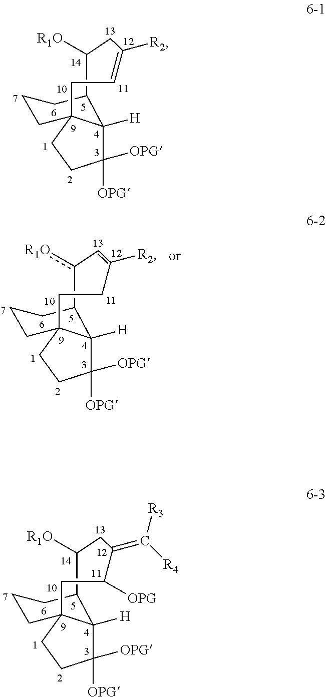
wherein the C14-O bond in formulas 6-1, 6-2, and 6-3 and the C11-O bond in formula 6-3 are independently selected from any stereoisomer orientation, R₁ is selected from the group consisting of H, H and a hydroxyl protecting group, any one of the diverse pleuromutilin or pleuromutilin derivative side-chains, and any one of the diverse pleuromutilin or pleuromutilin derivative side-chains having a protecting group, PG is a hydroxyl protecting group, PG′ is a ketone protecting group, and R2, R3 and R4 are independently selected from the group consisting of H, CH₃, an alkyl group, alkenyl group and an aryl group; and
one or more of i) introducing any one of the diverse pleuromutilin or pleuromutilin derivative side-chains as a C21 acyl side chain, ii) introducing one or more pleuromutilin analog substituent, and iii) conducting two fold de-protection to form a C3 ketone and unveil the C11 hydroxyl.

2. The method of claim 1, wherein the pleuromutilin skeleton has the structure of one of formulas 2-1a, 2-1aa, 2-1b, 2-1bb, 2-1, 4-1, or 4-1a:

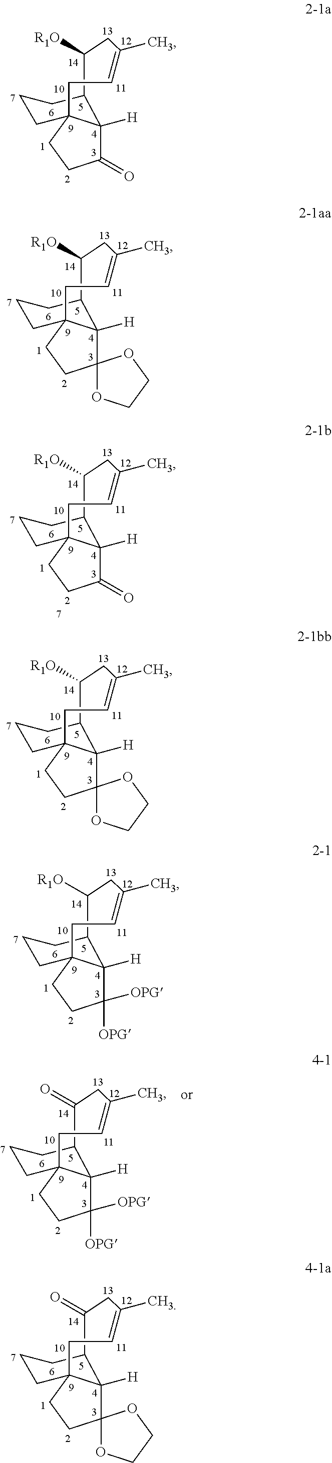

3. The method of claim 1, wherein the pleuromutilin skeleton has the structure of one of formulas 3-1a, 3-1 or 5-1:

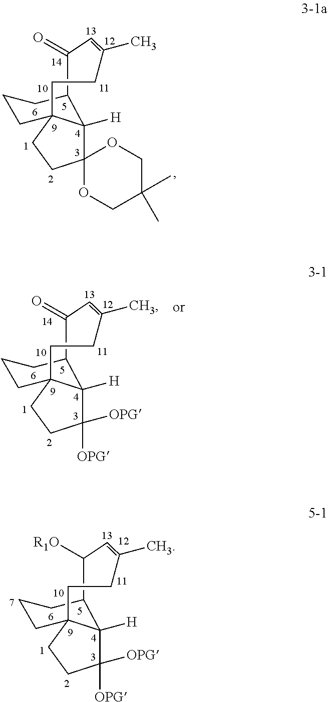

4. The method of claim 1, wherein the pleuromutilin skeleton has the structure of one of formulas 1-1a, 1-1b, 1-1c(1), 1-1c(2), or 1-1:

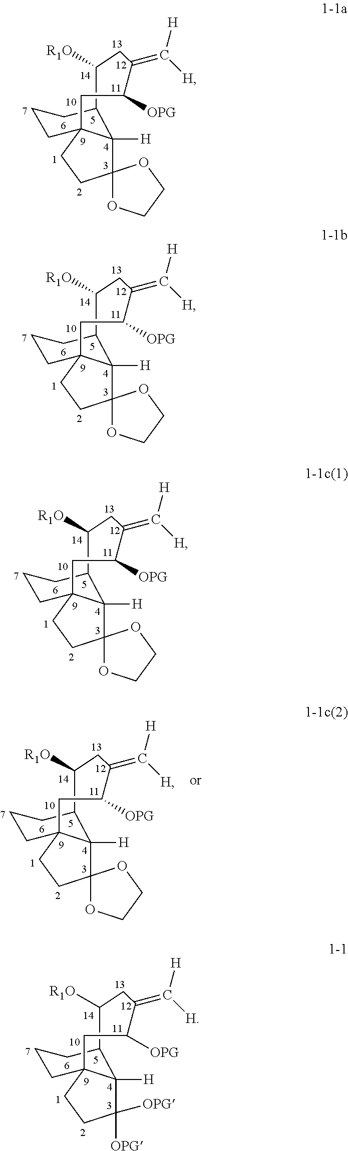

5. The method of claim 1, wherein the hydroxyl protecting group is selected from the group consisting of TBS, TES, TPS and MOM; the PG is selected from the group consisting of the ketone TBS, TMS, TES, TPS and MOM; and PG′ is selected from the group consisting of the ketals in the skeletons of formula 1-1a and 3-1a.

6. The method of claim 1, wherein providing the pleuromutilin skeleton comprises making the pleuromutilin the skeleton by a scheme including steps a), b) and c); and
the step a) comprises 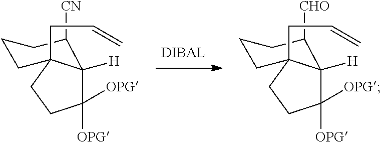
the step b) comprises 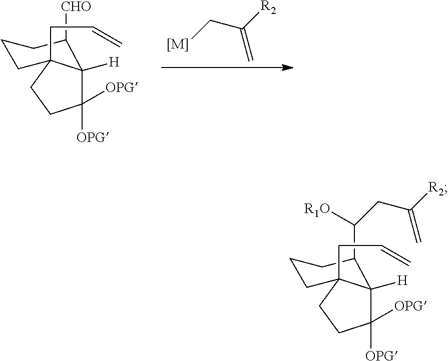
and
the step c) comprises 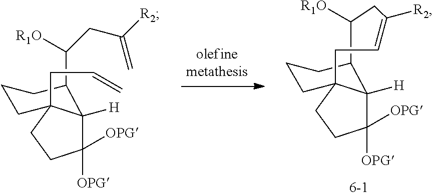
wherein M is a metal agent.

7. The method of claim 6, wherein M is selected from the group consisting of Zn, Mg, Li, Cr, and Sm.

8. The method of claim 1, wherein providing the pleuromutilin skeleton of formula 6-2 includes providing a pleuromutilin skeleton having the structure of formula 6-2a or 6-2b and comprises making the pleuromutilin the skeleton by a scheme including steps a), b), and c); and
the step a) comprises 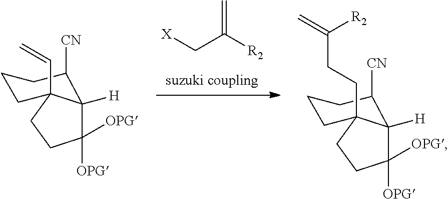
the step b) comprises one of
sub-scheme bi) comprising 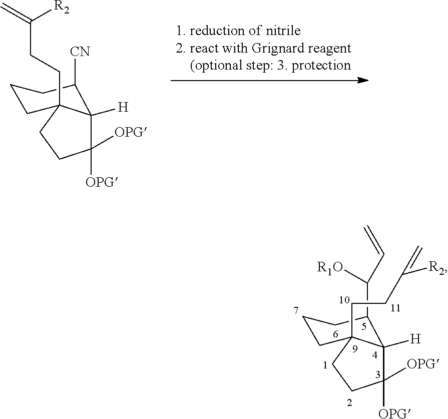
or
sub-scheme bii) comprising 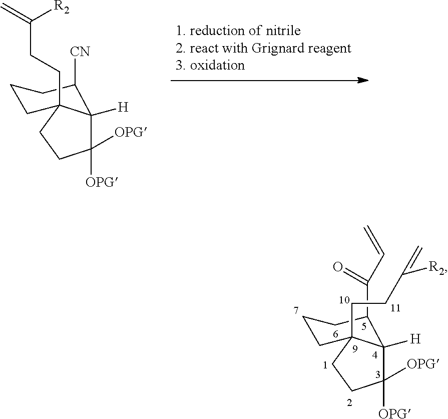
and
the step c) comprises olefin metathesis to produce the pleuromutilin skeleton of formula 6-2a from the product of sub-scheme bi) or the pleuromutilin skeleton of formula 6-2b form the product of sub-scheme bii):

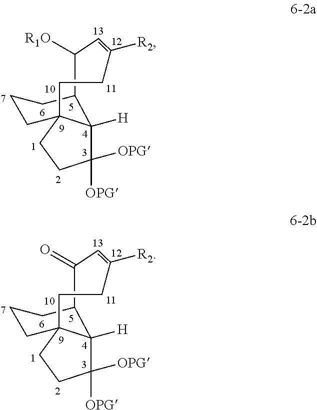

9. The method of claim 1, wherein providing the pleuromutilin skeleton comprises making the pleuromutilin the skeleton by a scheme including steps a), b) and c); and
the step a) comprises 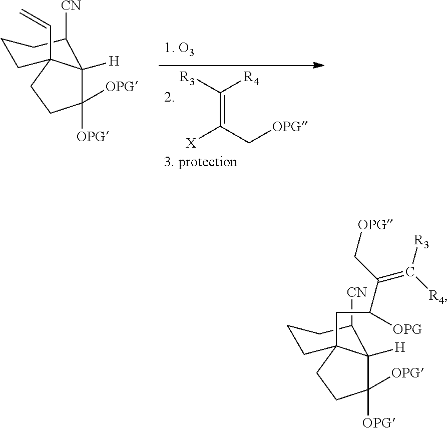
the step b) comprises 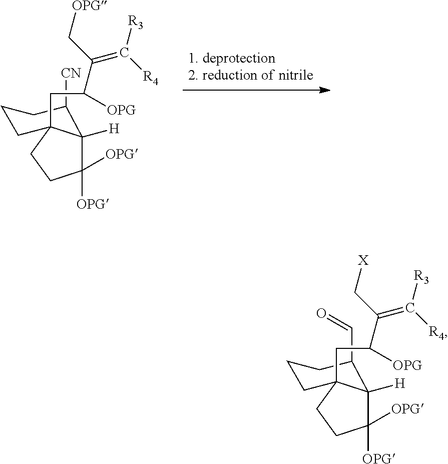
and
the step c) comprises 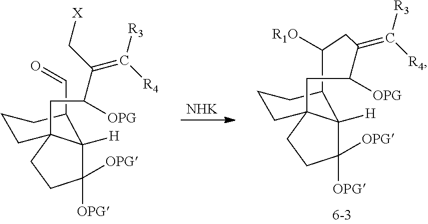
wherein PG″ is the same as PG, and X is a halide.

10. The method of claim 9, wherein X is selected from the group consisting of Cl, Br and I.

11. The method of claim 1, wherein providing the pleuromutilin skeleton comprises making the pleuromutilin the skeleton by a scheme including steps a) and b); and
the step a) comprises 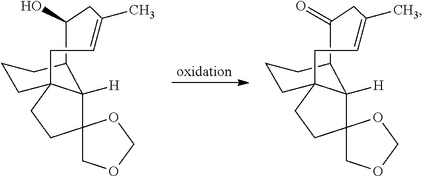
and
the step b) comprises

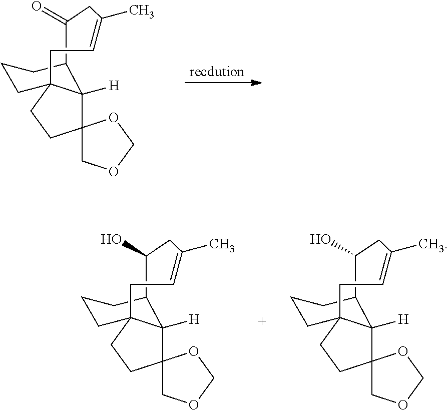

12. The method of claim 1, wherein the step of introducing one or more pleuromutilin analog substituent comprises dihyroxylation, epoxidation, hydroboration, ozonolysis, aziridination, difluorination, fluoride addition, epoxide opening, isomerization, or bromide addition, alpha-difluorination on the C12 alkene.

13. The method of claim 1, wherein the steps i) introducing any one of the diverse pleuromutilin or pleuromutilin derivative side-chains as a C21 acyl side chain, and ii) introducing one or more pleuromutilin analog substituent are conducted prior to the step of 11i) conducting two fold de-protection to form a C3 ketone and unveil the C11 hydroxyl and comprise one of the following schemes:
a) alkene epoxidation followed by C14 hydroxyl acylation;
b) alkene cyclopropanation followed by C14 hydroxyl acylation;
c) C14 hydroxyl acylation followed by alkene aziridination;
d) C14 hydroxyl acylation followed by singlet oxidation/Ph₃P;
e) alkene ozonolysis followed by Wittig reaction with R₁R₂C═PPh₃ and then C14 hydroxyl acylation;
f) alkene ozonolysis followed by C14 hydroxyl acylation;
g) C14 hydroxyl acylation followed by alkene hydroboration/oxidation;
h) alkene epoxidation followed by nucleophilic opening of epoxide then C14 hydroxyl acylation; or
i) C14 hydroxyl acylation followed by alkene dihydroxylation,
wherein for any of schemes a)-i) the C14 hydroxyl acylation is conducted to carry out the step of introducing any one of the diverse pleuromutilin or pleuromutilin derivative side-chains.

14. The method of claim 13, wherein the pleuromutilin skeleton has the structure of formula 6-3; R₁, R₃ and R₄ are H; and after the step of two fold de-protection scheme a) results in a compound having the structure of formula 51: 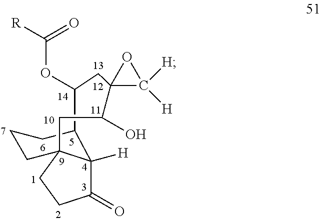
scheme b) results in a compound having the structure of formula 52: 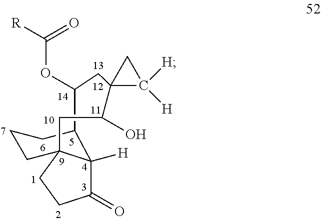
scheme c) results in a compound having the structure of formula 53: 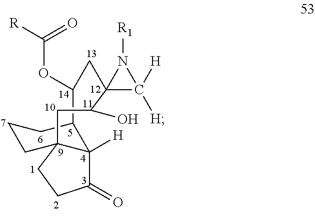
scheme d) results in a compound having the structure of formula 54: 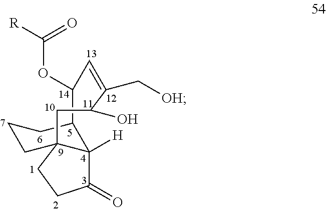
scheme e) results in a compound having the structure of formula 55: 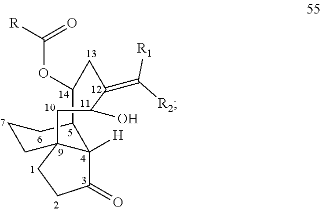
scheme f) results in a compound having the structure of formula 56: 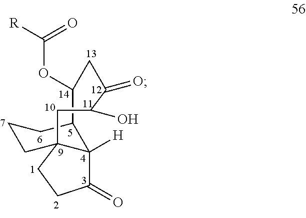
scheme g) results in a compound having the structure of formula 57: 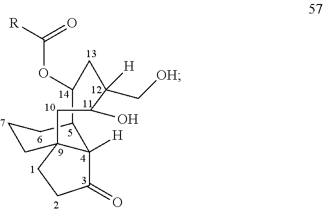
scheme h) results in a compound having the structure of formula 58: 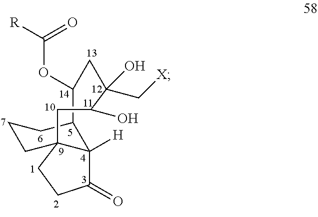
and
scheme i) results in a compound having the structure of formula 59:

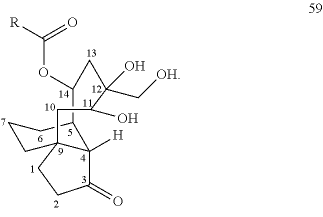

15. The method of claim 14, wherein X is selected from the group consisting of a nitrogen atom, a fluorine atom, an alkyl group, an alkenyl group and an aryl group.

16. A composition including a pleuromutilin analog having a structure of one formulas 7-1 or 7-2: 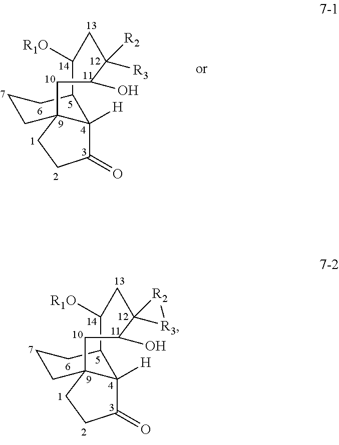
wherein the bonds on C11, C12 or C14 are independently selected from any stereoisomer orientation,
for the pleuromutilin analog having the structure of formula 7-1, R1 is selected from the group consisting of any one of the diverse pleuromutilin or pleuromutilin derivative side-chains as a C21 acyl side chain; R₂ is selected from the group consisting of H, OH, N and O; R₃ is selected from the group consisting of CH₂OH and CH₂X and X is selected from the group consisting of a halogen atom, a nitrogen atom, an alkyl group, an alkenyl group and an aryl group; and
for the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R₁ is selected from the group consisting of O, HN, HCH3, Net, NPr and NBu, and R₃ is selected from the group consisting of alkylene groups.

17. The composition of claim 16, wherein the diverse pleuromutilin or pleuromutilin derivative side-chains are selected from the group consisting of a tiamulin side chain, a pleuromutilin side chain and a retapamulin side chain.

18. The composition of claim 16, wherein the halogen is selected from the group consisting of fluorine, chlorine, bromine and iodine.

19. The composition of claim 16, wherein R₃ is CH₂ in the pleuromutilin analog having the structure of formula 7-2.

20. A pharmaceutical composition comprising a pleuromutilin analog or a pharmaceutically acceptable salt or solvate thereof, wherein the pleuromutilin analog has the structure of one of formulas 7-1 or 7-2: 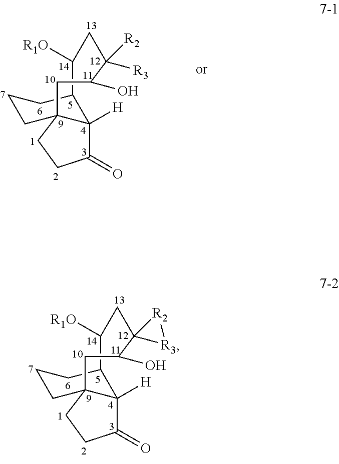
the bonds on C11, C12 or C14 are independently selected from any stereoisomer orientation,
for the pleuromutilin analog having the structure of formula 7-1, R₁ is selected from the group consisting of any one of the diverse pleuromutilin or pleuromutilin derivative side-chains as a C21 acyl side chain; R₂ is selected from the group consisting of H, OH, N and O; R₃ is selected from the group consisting of CH₂OH and CH₂X and X is selected from the group consisting of a halogen atom, a nitrogen atom, an alkyl group, an alkenyl group and an aryl group; and
for the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R₁ is selected from the group consisting of O, HN, HCH3, Net, NPr and NBu, and R₃ is selected from the group consisting alkylene groups.

21. The pharmaceutical composition of claim 20, wherein the diverse pleuromutilin or pleuromutilin derivative side-chains are selected from the group consisting of a tiamulin side chain, a pleuromutilin side chain and a retapamulin side chain.

22. The pharmaceutical composition of claim 20, wherein the halogen is selected from the group consisting of fluorine, chlorine, bromine and iodine.

23. The pharmaceutical composition of claim 20, wherein R₃ is CH₂ in the pleuromutilin analog having the structure of formula 7-2.

24. The pharmaceutical composition of claim 20 further comprising a pharmaceutically acceptable carrier.

25. A method of treating disease comprising administering a composition including a pleuromutilin analog or a pharmaceutical composition including a pleuromutilin analog or pharmaceutically acceptable salt or solvate thereof to a patient in need thereof, wherein the pleuromutilin analog has the structure of one of formulas 7-1 or 7-2: 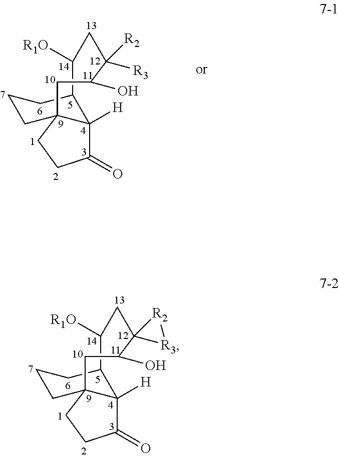
the bonds on C11, C12 or C14 are independently selected from any stereoisomer orientation,
for the pleuromutilin analog having the structure of formula 7-1, R₁ is selected from the group consisting of any one of the diverse pleuromutilin or pleuromutilin derivative side-chains as a C21 acyl side chain; R₂ is selected from the group consisting of H, OH, N and O; R₃ is selected from the group consisting of CH₂OH and CH₂X and X is selected from the group consisting of a halogen atom, a nitrogen atom, an alkyl group, an alkenyl group and an aryl group; and
for the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R₁ is selected from the group consisting of O, HN, HCH3, Net, NPr and NBu, and R₃ is selected from the group consisting of alkylene groups.

26. The method of claim 25, wherein the diverse pleuromutilin or pleuromutilin derivative side-chains are selected from the group consisting of a tiamulin side chain, a pleuromutilin side chain and a retapamulin side chain; the halogen is selected from the group consisting of fluorine, chlorine, bromine and iodine; and R₃ is CH₂ in the pleuromutilin having the structure of formula 7-2.

27. The method of claim 25, wherein the disease is selected from the group consisting of a microbial infection, an infection by bacteria, an infection by gram negative bacteria, an infection by Staphylococcus, an infection by Staphylococcus aureous, an infection by Staphylococcus pyogenes, an infection by Mycobacterium, an infection by Mycobacterium tuberculosis, tuberculosis, a skin infection and a lung infection.

28. A method of analyzing the affect of point mutations within a pleuromutilin compound comprising:
exposing a Mycobacterium tuberculosis model organism to a pleuromutilin analog, wherein the pleuromutilin analog has the structure of one of formulas 7-1 or 7-2: 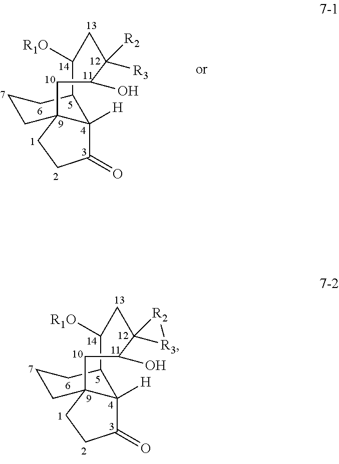
the bonds on C11, C12 or C14 are independently selected from any stereoisomer orientation,
for the pleuromutilin analog having the structure of formula 7-1, R₁ is selected from the group consisting of any one of the diverse pleuromutilin or pleuromutilin derivative side-chains as a C21 acyl side chain; R₂ is selected from the group consisting of H, OH, N and O; R₃ is selected from the group consisting of CH₂OH and CH₂X and X is selected from the group consisting of a halogen atom, a nitrogen atom, an alkyl group, an alkenyl group and an aryl group; and
for the pleuromutilin analog having the structure of formula 7-2, R₁ is selected from the group consisting of any one of the diverse pleuromutilin or pleuromutilin derivative side-chains as a C21 acyl side chain, and R₂ and R₃ are selected from the group consisting of alkylene groups; or R₁ is selected from the group consisting of O, HN, HCH3, Net, NPr and NBu, and R₃ is selected from the group consisting of alkylene groups.

29. The method of claim 28, wherein the diverse pleuromutilin or pleuromutilin derivative side-chains are selected from the group consisting of a tiamulin side chain, a pleuromutilin side chain and a retapamulin side chain; the halogen is selected from the group consisting of fluorine, chlorine, bromine and iodine; and R₃ is CH₂ in the pleuromutilin having the structure of formula 7-2.

30. A composition comprising a pleuromutilin skeleton having the structure of one of formulas 6-1, 6-2 or 6-3: 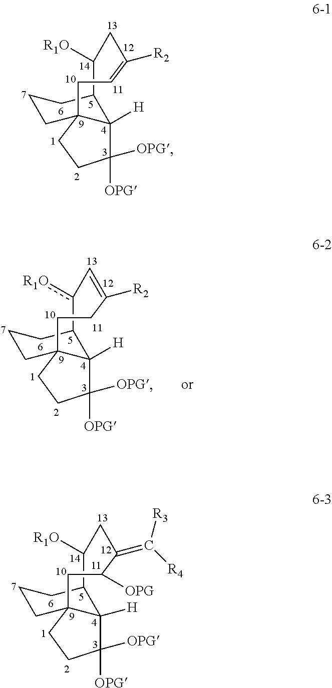
wherein the C14-O bond in formulas 6-1, 6-2, and 6-3 and the C11-O bond in formula 6-3 are independently selected from any stereoisomer orientation, R₁ is selected from the group consisting of H, H and a hydroxyl protecting group, any one of the diverse pleuromutilin or pleuromutilin derivative side-chains, and any one of the diverse pleuromutilin or pleuromutilin derivative side-chains and a protecting group, PG is a hydroxyl protecting group, PG′ is a ketone protecting group, and R₂, R₃ and R₄ are independently selected from the group consisting of H, CH₃, an alkyl group, alkenyl group and an aryl group.

31. The composition of claim 30, wherein the pleuromutilin skeleton has the structure of one of formulas 2-1a, 2-1aa, 2-1b, 2-1bb, 2-1, 4-1, or 4-1a:

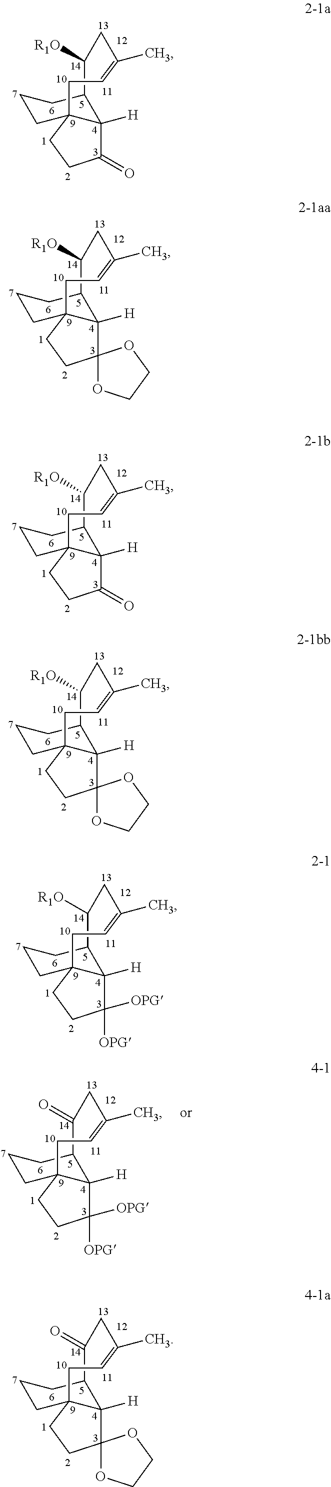

32. The composition of claim 30, wherein the pleuromutilin skeleton has the structure of one of formulas 3-1a, 3-1 or 5-1:

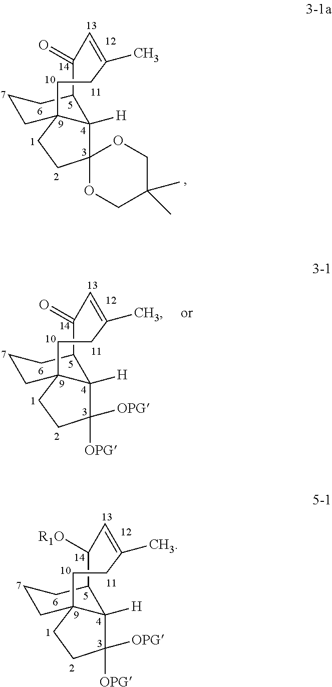

33. The composition of claim 30, wherein the pleuromutilin skeleton has the structure of one of formulas 1-1a, 1-1b, 1-1c(1), 1-1c(2), or 1-1: [chemical structure]
